(19) United States
(12) Patent Application Publication
(10) Pub. No.: US 2007/0105109 A1
(43) Pub. Date: May 10, 2007

(54) Sirt1 and genetic disorders

(76) Inventors: Bard J. Geesaman, Cambridge, MA (US); Laura Brass, Cambridge, MA (US)

Correspondence Address: FISH & RICHARDSON PC, P.O. BOX 1022, MINNEAPOLIS, MN, 55440-1022, US

(21) Appl. No.: 10/562,389

(22) PCT Filed: Jul. 1, 2004
(86) PCT No.: PCT/US04/21189

§ 371 Date: Jun. 30, 2006

Related U.S. Application Data
(60) Provisional application No. 60/484,327, filed on Jul. 2, 2003.

(51) Int. Cl. C12Q 1/68; G06F 19/00

(52) U.S. Cl. 435/6; 702/20

(57) ABSTRACT

Alzheimer's Disease (AD) is genetically linked to a locus that includes the SIRT1 gene. This disclosure includes methods and compositions for evaluating genetic variances, e.g., within this locus, and for the association of these variances with AD. The disclosure also includes methods and compositions for modulating SIRT1 expression and/or activity. These methods and compositions can be used in the prevention or treatment of AD.

### CROSS-REFERENCE TO RELATED APPLICATIONS

This application claims priority to U.S. Application Ser. No. 60/484,327, filed on Jul. 2, 2003, the contents of which are hereby incorporated by reference in its entirety.

### BACKGROUND

Alzheimer's Disease is a complex neurodegenerative disease that results in the irreversible loss of neurons. Clinical hallmarks of Alzheimer's Disease (AD) include progressive impairment in memory, judgment, orientation to physical surroundings, and language. Neuropathological hallmarks of AD include region-specific neuronal loss, amyloid plaques, and neurofibrillary tangles. Amyloid plaques are extracellular plaques containing the β amyloid peptide (also known as Aβ, or Aβ42), which is a cleavage product of the β-amyloid precursor protein (also known as APP). Neurofibrillary tangles are insoluble intracellular aggregates composed of filaments of the abnormally hyperphosphorylated microtubule-associated protein, tau. Amyloid plaques and neurofibrillary tangles may contribute to secondary events that lead to neuronal loss by apoptosis (Clark and Karlawish, Ann. Intern. Med. 138(5):400-410 (2003). For example, β-amyloid induces caspase-2-dependent apoptosis in cultured neurons (Troy et al. J. Neurosci. 20(4):1386-1392). The deposition of plaques in vivo may trigger apoptosis of proximal neurons in a similar manner.

Mutations in genes encoding APP, presenilin-1, and presenilin-2 have been implicated in early-onset AD (Lendon et al. JAMA 227:825 (1997)). Mutations in these proteins have been shown to enhance proteolytic processing of APP via an intracellular pathway that produces Aβ. Aberrant regulation of Aβ processing may be central to the formation of amyloid plaques and the consequent neuronal damage associated with plaques.

Further, inheritance of a particular allele of the ApoE gene correlates with an increased lifetime risk for AD (Strittmatter and Roses. Annu. Rev. Neurosci. 19:53-77 (1996)). Also, a 17 million base-pair genetic locus on chromosome 10q has been implicated in late-onset AD (Ertekin-Taner et al. Science 290:23032304 (2000); Myers et al. Science 290:2304-2305 (2000)).

### SUMMARY

In one aspect, this disclosure features a method that includes genotyping a human gene that encodes a sirtuin, e.g., SIRT1 or another sirtuin and recording information about the genotype in association with information about a sirtuin-associated disorder, e.g., a SIRT1-associated disorder, or Alzheimer's disease. Other examples of sirtuins include: SIRT2, SIRT3, SIRT4, SIRT5, SIRT6, and SIRT7.

The disclosure also features a method that includes genotyping a human gene that encodes a sirtuin, e.g., SIRT1 or another SIRT1 and recording information about the genotype in association with information about an age-related disorder. Exemplary age-related disorders are provided below.

In one aspect, this disclosure features a method that includes a) determining the identity of at least one nucleotide in the SIRT1 locus on human chromosome 10q of a subject; and b) creating a record which includes information about the identity of the nucleotide and information relating to an Alzheimer's Disease (AD)-related parameter of the subject, wherein the AD-related parameter is other than the genotype of a nucleotide in the 10q AD6 region. The method can be used, e.g., for gathering genetic information. In one embodiment, the determining includes evaluating a sample including human genetic material from the subject.

Another method includes: a) evaluating a parameter of a SIRT1 molecule from a mammalian subject; b) evaluating an Alzheimer's Disease (AD)-related parameter of the subject wherein the AD-related parameter is other than a parameter of a SIRT1 molecule; and c) recording information about the SIRT1 parameter and information about the AD-related parameter, wherein the information about the parameter and information about the phenotypic trait are associated with each other in the database. For example, the AD-related parameter is a phenotypic trait of the subject.

In one embodiment, the SIRT1 molecule is a polypeptide and the SIRT1 parameter includes information about a SIRT1 polypeptide. In another embodiment, the SIRT1 molecule is a nucleic acid and the SIRT1 parameter includes information about identity of a nucleotide in the SIRT1 gene or a gene or other sequence located between the gene DKFZP564G092 and LOC283055( hypothetical gene supported by NM_{—}000976; AK026491; L06505).

In an embodiment, the subject is an embryo, blastocyst, or fetus. In another embodiment, the subject is a post-natal human, e.g., a child or an adult (e.g., at least 20, 30, 40, 50, 60, 70 years of age).

In one embodiment, step b) is performed before or concurrent with step a). In one embodiment, the human genetic material includes DNA and/or RNA.

The method can further include comparing the SIRT1 parameter to reference information, e.g., information about a corresponding nucleotide from a reference sequence. For example, the reference sequence is from a reference subject who has attained old age, e.g., at least 85, 90, 95, 98, 100, 102, or 105 years of age. In one embodiment, the reference subject did not exhibit AD, e.g., at least prior to the time at which a nucleic acid from the reference subject was obtained or at least prior to 85, 90, 95, 98, 100, 102, or 105 years of age. In one embodiment, the reference subject was cognitively intact, e.g., at least prior to the time at which a nucleic acid from the reference subject was obtained or at least prior to 85, 90, 95, 98, 100, 102, or 105 years of age. In another embodiment, the reference sequence is from a reference subject that has AD, e.g., early or late-onset AD (LOAD).

In one embodiment, the method further includes comparing the nucleotide to a corresponding nucleotide from a genetic relative or family member (e.g., a parent, grandparent, sibling, progeny, prospective spouse, etc.).

In one embodiment, the method further includes evaluating risk or determining diagnosis of AD in the subject as a function of the genotype.

In one embodiment, the method further includes recording information about the SIRT1 parameter and AD-related parameter, e.g., in a database. For example, the information is recorded in linked fields of a database (e.g., SIRT1 parameter is linked to at least one of: corresponding SIRT1 parameter and/or data regarding comparison with the reference sequence). The nucleotide can be located in an exon, intron, or regulatory region of the SIRT1 gene. For example, the nucleotide is a SNP. The identity of at least one SNP from Table 1 can be evaluated. In one embodiment, a plurality of nucleotides (e.g., at least 10, 20, 50, 100, 500, or 1000 nucleotides are evaluated (e.g., consecutive or non-consecutive)) in the SIRT1 locus are evaluated. In another embodiment, a single nucleotide is evaluated.

In one embodiment, the method includes one or more of: evaluating a nucleotide position in the SIRT1 locus on both chromosomes of the subject; recording the information (e.g., as phased or unphased information); aligning the genotyped nucleotides of the sample and the reference sequence; and identifying nucleotides that differ between the subject nucleotides and the reference sequence.

The method can be repeated for a plurality of subjects (e.g., at least 10, 25, 50, 100, 250, 500 subjects).

In one embodiment, the method can include comparing the information of step a) and step b) to information in a database, and evaluating the association of the genotyped nucleotide(s) with AD.

In one embodiment, the AD-related parameter is a biochemical parameter, e.g., an assessment of IGF-1, Ab42, tau, or vitamin B12. For example, the assessment is of plasma, serum or cerebrospinal fluid (CSF). Another biochemical parameter includes information about plasma Ab42 levels. For example, the evaluating of an AD-related parameter includes an immuno-assay. Other features that can be evaluated include 8-hydroxyguanine levels in CSF(e.g., Ab42, tau protein); F2 isoprostane levels in CSF, plasma, and/or urine (e.g., urine NT? (neural thread protein). Isoprostane, 8,12-iso-iPf2^{a}-VI, is indicative of brain oxidative damage and is elevated in the spinal fluid, blood, and urine of patients with mild cognitive impairment (MCI), which may precede Alzheimer's disease

In another embodiment, the AD-related parameter is an assessment of cognitive function. The AD-related parameter includes a result of a mental examination (e.g., a Folstein Mini-Mental Status Examination), a memory test, a behavioral test, a personality test, or other cognitive test. For example, the AD-related parameter includes information about a symptom of dementia. For example, the symptom of dementia includes at least one of the following: decline in mental status (e.g., as assessed by the Folstein Mini-Mental Status Examination, or the Barthel Scale or other equivalent); loss of recent memory; inability to learn and remember new information; behavioral disorganization; diminished abstract thinking; diminished judgment; and personality changes (e.g., mood swings, irritability).

In one embodiment, the AD-related parameter is an anatomical feature. The AD-related parameter includes information about one or more of the following: a brain lesion or brain atrophy (e.g., bilateral asymmetric hypoperfusion in the parietal and temporal lobes as determined by imaging, e.g., MRI or computed tomography). For example, the AD-related parameter includes information about a genetic polymorphism associated with AD other than a nucleotide polymorphism present in the SIRT1 locus. For example, the genetic polymorphism is a polymorphism of a gene encoding: ApoE, presenilin 1, presenilin 2, or APP.

The genetic polymorphism can be a nucleotide polymorphism, e.g., a SNP.

The method can further include making a decision about whether to provide an AD treatment as a function of the SIRT1 parameter.

In another aspect, this disclosure features a computer-readable database that includes a plurality of records. Each record includes a) a first field which includes information about one or more nucleotides from a SIRT1 locus of a subject and; b) a second field which includes information about AD-related parameter of the subject. For example, the AD-related parameter includes information about a biochemical feature, anatomical feature, or cognitive assessment. For example,, the AD-related parameter is an AD diagnosis.

A related database has records that each include a) a first field which includes information about one or more nucleotides from a locus that encodes a sirtuin (e.g., a human sirtuin) of a subject and; b) a second field which includes information about a parameter that is associated with an age-associated disease of the subject.

In another aspect, this disclosure features a method that includes: a) genotyping (e.g., determining the identity of) one or more nucleotides from a sample from a human subject, wherein the nucleotides are in a gene of a human SIR2 homolog or sirtuin; and b) evaluating one or more features of Alzheimer's Disease (AD) in the subject, or one or more features of another age-associated disorder. The method can include other features described herein.

In another aspect, this disclosure features a method that includes a) determining the identity of at least one nucleotide in the SIRT1 locus on human chromosome 10q for a plurality of subjects who have AD or are associated with AD; and b) evaluating the distribution of one or more nucleotide identities for a given position in the SIRT1 locus among or between subjects of the plurality. In one embodiment, evaluating the distribution further includes comparing one or more nucleotide identities to corresponding nucleotides in subjects who do not have AD or who are not associated with AD. The method can include other features described herein.

In another aspect, this disclosure features a method for evaluating a compound. The method includes: evaluating a compound for an effect on SIRT1 activity; and evaluating a compound for an effect on AD. Similarly it is possible to evaluate a plurality of compounds (e.g., from a library of compounds). For each compound of a plurality of compounds, the method includes evaluating the compound for an effect on SIRT1 activity; and, optionally if the compound has an effect on SIRT1 activity, evaluating the compound for an effect on AD.

In one embodiment, evaluating for an effect on SIRT1 activity includes evaluating SIRT1 mRNA expression. In another embodiment, evaluating for an effect on SIRT1 activity includes evaluating a SIRT1 polypeptide (e.g., evaluating SIRT1 enzymatic activity, e.g., deacetylase activity). For example, evaluating for an effect on SIRT1 activity includes evaluating deacetylase activity for a SIRT1 specific substrate, e.g., an acetylated lysine amino acid, an acetylated peptide or acetylated protein. For example, the acetylated peptide or acetylated protein includes an acetylated amino acid sequence of at least 6, 7, 8, or 10 amino acid from a histone (e.g., an N-terminal tail) or other SIRT1 interaction partner, e.g., p53 or FOXO, e.g., FOXO4, re1A/p65, or bHLH repressors HES1 and HEY2.

In one embodiment, evaluating for an effect on AD includes contacting the agent to a neuronal cell.

In one embodiment, evaluating for an effect on AD includes contacting the agent to a mammal, e.g., a mouse model of AD. For example, evaluating for an effect on AD includes testing the mammal with a cognitive test or evaluating the mammal for tangle formation.

The method (using a cell or organism) can include evaluating a secretase protein or mRNA or evaluating secretase activity. The method (using a cell or organism) can include evaluating a APP or a fragment thereof.

In another aspect, this disclosure features a method that includes providing a computer model of the structure of a compound and the structure of a sirtuin (e.g., SIRT1 protein); evaluating compatibility of the models; and evaluating a compound for an effect on AD.

For example, evaluating model compatibility includes evaluating an energy potential or steric compatibility. The method can include other features described herein.

In another aspect, this disclosure features a method for treating or preventing Alzheimer's Disease (AD) in a subject. The method can include: identifying a subject diagnosed with or at risk for AD; and administering to the subject an agent that modulates SIRT1 activity. For example, the agent is administered in an amount effective to reduce apoptosis in the subject, to reduce amyloid plaque formation in the subject, or to reduce or ameliorate at least one symptom of AD. In one embodiment, the agent increases SIRT1 activity.

The agent can be an agent which increases SIRT1 activity, e.g., at least 0.5, 1, 2, 3, 4, 8, 10, or 12-fold, e.g., between 2 and 15-fold. For example, the agent can be a polyphenol, e.g., a flavone, stilbene, flavanone, isoflavones, catechins, chalcone, tannin, or anthocyanidin. For example, the agent is a trans-stilbene, e.g., resveratrol. The agent may also be a nucleic acid that encodes a SIRT1 polypeptide or a functional domain thereof, e.g., the core domain. The core domain of human SIRT1 is from amino acids 214-541 of the 747 amino acid protein, SEQ ID NO:2. The agent may be prepared, e.g., using a synthetic process or from a natural product, e.g., by extraction from a natural product.

For example, if the agent is a trans-stilbene such as resveratrol, either synthetically made or made from a natural product, the agent can be administered in a dosage of at least 0.5, 1, 5, 10, 20, 50, or 100 mg per day to a subject, e.g., a human subject.

For example, the identifying includes evaluating a feature for AD in the subject (e.g., a genetic, biochemical, anatomical, or cognitive feature or a symptom of AD). In one embodiment, the feature of AD is a genetic polymorphism associated with AD, e.g., in the ApoE locus or in the SIRT1 gene. For example, the method can be used to administer the agent to subjects that have an uncommon allele or AD-associated allele of the SIRT1 gene.

In one embodiment, the identifying includes evaluating one or more nucleotides in a SIRT1 nucleic acid of the subject (e.g., in the SIRT1 gene in the genome of the subject or in a SIRT1 RNA or cDNA).

It may also be possible to use agents which modulate other sirtuin activities to ameliorate at least one symptom of other age-related disorders.

In another aspect, this disclosure features a method for reducing AD-induced apoptosis in a cell. The method includes contacting the cell with an agent that increases SIRT1 activity (e.g., in vivo or in vitro). For example, the cell is at risk for AD or from a subject at risk for or diagnosed with AD. For example, the agent includes a nucleic acid that encodes a SIRT1 polypeptide. In another example, the agent is an siRNA that inhibits an inhibitor of SIRT1. The agent can be an agent described herein or an agent identified by a method described herein. The method can include other features described herein.

Some Definitions

An “allele” refers to a particular genetic variation in a nucleic acid sequence. Such variation can be present in a gene or outside of a gene. For example, the variation can be present in a coding, non-coding, regulatory, or non-functional region of a nucleic acid sequence. Variations can be present in euchromatin or heterochromatin and so forth.

As used herein, the term “polymorphism” generally refers to any variation in sequence at a given position or region of nucleic acid sequence between individuals in a population, e.g., human individuals. Variations include nucleotide substitutions (e.g., transitions and transversions), insertions, deletions, inversions, and other rearrangements. A variation can encompass one or more nucleotide positions in a reference sequence that are absent, altered, inverted, or otherwise rearranged in another sequence. Some exemplary polymorphisms cause one or more changes in the amino acid sequence of an encoded protein. Other exemplary polymorphisms can affect regulation, e.g., transcription, translation, splicing, mRNA or protein stability, mRNA or protein localization, chromatin organization, and so forth. Still other exemplary polymorphisms are silent or are only manifest under particular circumstances. Even completely silent markers are useful, e.g., as indicators. For example, they may be tightly linked to a marker that is causative of a particular property. Typically a polymorphic marker described herein is an inherited variant, but may also arise through a spontaneous recombination event, or by artificial means, e.g., by a targeted genetic manipulation.

As used herein, “genotyping” refers to any method of evaluating genetic material. Genotyping includes a method of determining the identity of one or more nucleotides (a consecutive or non-consecutive positions), sequencing a region of nucleic acid, and determining the type and number of alleles and/or polymorphisms present in genetic material, e.g., genetic material from a subject. Exemplary methods of genotyping determined by nucleic acid sequencing, PCR or RT-PCR amplification, protein sequencing (thereby inferring nucleic acid sequence), examination of a protein, or by other methods available to those skilled in the art.

As used herein, the term “nucleic acid molecule” includes DNA molecules (e.g., a cDNA or genomic DNA), RNA molecules (e.g., an mRNA, a dsRNA, e.g., an siRNA) and analogs of the DNA or RNA. A DNA or RNA analog can be synthesized from nucleotide analogs. The nucleic acid molecule can be single-stranded or double-stranded, e.g., double-stranded DNA or a double-stranded RNA.

The term “isolated nucleic acid molecule” or “purified nucleic acid molecule” includes nucleic acid molecules that are separated from other nucleic acid molecules present in the natural source of the nucleic acid. For example, an isolated nucleic acid can be at least 10, 20, 40, 50, 60, 70, 80, or 90% pure, e.g., more than 99% pure. For example, with regards to genomic DNA, the term “isolated” includes nucleic acid molecules which are separated from the chromosome with which the genomic DNA is naturally associated. In some embodiments, an “isolated” nucleic acid is free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which naturally flank the nucleic acid molecule in genomic DNA of the cell from which the nucleic acid is derived. Examples of flanking sequences include adjacent genes, transposons, and regulatory sequences. Moreover, an “isolated” nucleic acid molecule, such as a cDNA molecule, can be substantially free of other cellular material, of culture medium when produced by recombinant techniques, or of chemical precursors or other chemicals when chemically synthesized.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2× SSC, 0.1% SDS at least at 50° C. (the temperature of the washes can be increased to 55° C. for low stringency conditions); 2) medium stringency hybridization conditions in 6× SSC at about 45° C., followed by one or more washes in 0.2× SSC, 0.1% SDS at 60° C.; 3) high stringency hybridization conditions in 6× SSC at about 45° C., followed by one or more washes in 0.2× SSC, 0.1% SDS at 65° C.; and preferably 4) very high stringency hybridization conditions are 0.5 M sodium phosphate, 7% SDS at 65° C., followed by one or more washes at 0.2× SSC, 1% SDS at 65° C. Very high stringency conditions (4) are the preferred conditions and the ones that should be used unless otherwise specified. Methods described herein can include use of an isolated nucleic acid molecule that hybridizes under a stringency condition described herein to a sequence described herein or use of a polypeptide encoded by such a sequence, e.g., the molecule can be a naturally occurring variant.

As used herein, a “naturally-occurring” nucleic acid molecule refers to an RNA or DNA molecule having a nucleotide sequence that occurs in Nature. For example, a naturally occurring nucleic acid molecule can encode a natural protein.

As used herein, the terms “gene” and “recombinant gene” refer to nucleic acid molecules which include at least an open reading frame encoding a protein or subunit, derivative, or functional domain thereof. The gene can optionally further include non-coding sequences, e.g., regulatory sequences (e.g., transcriptional and translational regulatory sequences) and introns. Accordingly, the open reading frame can be interrupted by one or more introns. Some regulatory sequences can be quite distant, depending on the gene and, e.g., chromosomal organization.

The term “polypeptide” refers to a polymer of three or more amino acids linked by a peptide bond. The polypeptide may include one or more unnatural amino acids. Typically, the polypeptide includes only natural amino acids. The term “peptide” refers to a polypeptide that is between three and thirty-two amino acids in length. A protein can include one or more polypeptide chains. A polypeptide may include one or more unnatural amino acids. Typically, the polypeptide includes only natural amino acids.

A protein or polypeptide can also include one or more modifications, e.g., a glycosylation, amidation, phosphorylation, and so forth.

An “isolated” or “purified” polypeptide or protein is substantially free of cellular material or other contaminating proteins from the cell or tissue source from which the protein is derived, or substantially free from chemical precursors or other chemicals when chemically synthesized. “Substantially free” means that the protein of interest in the preparation is at least 10% pure. In an embodiment, the preparation of the protein has less than about 30%, 20%, 10% and more preferably 5% (by dry weight), of a contaminating component (e.g., a protein not of interest, chemical precursors, and so forth). When the protein or biologically active portion thereof is recombinantly produced, it is also preferably substantially free of culture medium, i.e., culture medium represents less than about 20%, more preferably less than about 10%, and most preferably less than about 5% of the volume of the protein preparation. The disclosure includes isolated or purified preparations of at least 0.01, 0.1, 1.0, and 10 milligrams in dry weight.

A “non-essential” amino acid residue is a residue that can be altered from the wild-type sequence of protein without abolishing or substantially altering activity, e.g., the activity is at least 20%, 40%, 60%, 70% or 80% of wild-type. An “essential” amino acid residue is a residue that, when altered from the wild-type sequence results in abolishing activity such that less than 20% of the wild-type activity is present. Conserved amino acid residues are frequently predicted to be particularly unamenable to alteration.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a protein is preferably replaced with another amino acid residue from the same side chain family. Alternatively, in another embodiment, mutations can be introduced randomly along all or part of a coding sequence, such as by saturation mutagenesis, and the resultant mutants can be screened for biological activity to identify mutants that retain activity. Following mutagenesis, the encoded protein can be expressed recombinantly and the activity of the protein can be determined.

As used herein, a “biologically active portion” or a “functional domain” of a protein includes a fragment of a protein of interest which participates in an interaction, e.g., an intramolecular or an inter-molecular interaction, e.g., a binding or catalytic interaction. An inter-molecular interaction can be a specific binding interaction or an enzymatic interaction (e.g., the interaction can be transient and a covalent bond is formed or broken). An inter-molecular interaction can be between the protein and another protein, between the protein and another compound, or between a first molecule and a second molecule of the protein (e.g., a dimerization interaction). Biologically active portions/functional domains of a protein include peptides comprising amino acid sequences sufficiently homologous to or derived from the amino acid sequence of the protein which include fewer amino acids than the full length, natural protein, and exhibit at least one activity of the natural protein. Biological active portions/functional domains can be identified by a variety of techniques including truncation analysis, site-directed mutagenesis, and proteolysis. Mutants or proteolytic fragments can be assayed for activity by an appropriate biochemical or biological (e.g., genetic) assay. In some embodiments, a functional domain is independently folded. Typically, biologically active portions comprise a domain or motif with at least one activity of a protein, e.g., SIRT1 (also discussed below). An exemplary domain is the SIRT1 core domain. The core domain of human SIRT1 is from amino acids 214-541 of the 747 amino acid protein, SEQ ID NO:2. A biologically active portion/functional domain of a protein can be a polypeptide which is, for example, 10, 25, 50, 100, 200 or more amino acids in length. Biologically active portions/functional domain of a protein can be used as targets for developing agents which modulate SIRT1.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, 50%, 60%, 70%, 80%, 90%, 95% or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using the NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of Meyers and Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

Some polypeptides can have an amino acid sequence substantially identical to an amino acid sequence described herein. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. Methods described herein can include use of a polypeptide that includes an amino acid sequence that contains a structural domain having at least about 60%, or 65% identity, likely 75% identity, more likely 85%, 90%, 92%, 94%, 95%, 96%, 97%, 98% or 99% identity to a domain of a polypeptide described herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. Methods described herein can include use of a nucleic acid that includes a region at least about 60%, or 65% identity, likely 75% identity, more likely 85%, 90%, 91%, 92%, 93%, 94%, 95%, 96%, 97%, 98% or 99% identity to a nucleic acid sequence described herein, or use of a protein encoded by such nucleic acid.

“Sirtuins” are proteins that include a SIR2 domain, a domain defined as amino acids sequences that are scored as hits in the Pfam family “SIR2”- PF02146 (attached to the Appendix). This family is referenced in the INTERPRO database as INTERPRO description (entry IPR003000). To identify the presence of a “SIR2” domain in a protein sequence, and make the determination that a polypeptide or protein of interest has a particular profile, the amino acid sequence of the protein can be searched against the Pfam database of HMMs (e.g., the Pfam database, release 9) using the default parameters (http://www.sanger.ac.uk/Software/Pfam/HMM_search). The SIR2 domain is indexed in Pfam as PF02146 and in INTERPRO as INTERPRO description (entry IPR003000). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in “The Pfam Protein Families Database” Bateman A, Birney E, Cerruti L, Durbin R, Etwiller L, Eddy S R, Griffiths-Jones S, Howe K L, Marshall M, Sonnhammer E L (2002) Nucleic Acids Research 30(l):276-280 and Sonhammer et al. (1997) Proteins 28(3):405-420 and a detailed description of HMMs can be found, for example, in Gribskov et al.(1990) Meth. Enzymol. 183:146-159; Gribskov et al.(1987) Proc. Natl. Acad. Sci. USA 84:4355-4358; Krogh et al.(1994) J. Mol. Biol. 235:1501-1531; and Stultz et al.(1993) Protein Sci. 2:305-314.

A “purified preparation of cells”, as used herein, refers to an in vitro preparation of cells. In the case of cells from multicellular organisms (e.g., plants and animals), a purified preparation of cells is a subset of cells obtained from the organism, not the entire intact organism. In the case of unicellular microorganisms (e.g., cultured cells and microbial cells), such a preparation consists of a plurality of cells, of which at least 10% and more preferably 50% are constituted by the subject cells.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “small organic molecule” is an organic molecule of having a molecular weight of less than 5, 2, 1, or 0.5 kDa. In many embodiments, such small molecules do not include a peptide bond or a phosphodiester bond. For example, they can be non-polymeric. In some embodiments, the molecule has a molecular weight of at least 50, 100, 200, or 400 Daltons.

“Binding affinity” refers to the apparent dissociation constant or K_{D}. A ligand may, for example, have a binding affinity (K_{D}) of less than 10⁻⁵, 10⁻⁶, 10⁻⁷ or ⁻⁸ M for a particular target molecule. Higher affinity binding of a ligand to a first target relative to a second target can be indicated by a smaller numerical value K_{D}¹ for binding the first target than the numerical value KD² for binding the second target. In such cases the ligand has specificity for the first target relative to the second target. The agent may bind specifically to the target, e.g., with an affinity that is at least 2, 5, 10, 100, or 1000 better than for a non-target. For example, an agent can bind to SIRT1 with a K_{d} of less than 10⁻⁵, 10⁻⁶, 10⁻⁷ or 10⁻⁸ M in PBS.

Binding affinity can be determined by a variety of methods including equilibrium dialysis, equilibrium binding, gel filtration, ELISA, or spectroscopy (e.g., using a fluorescence assay). These techniques can be used to measure the concentration of bound and free ligand as a function of ligand (or target) concentration. The concentration of bound ligand ([Bound]) is related to the concentration of free ligand ([Free]) and the concentration of binding sites for the ligand on the target where (N) is the number of binding sites per target molecule by the following equation:
[Bound]=N·[Free]/((1/Ka)+[Free])

The details of one or more embodiments described herein are set forth in the accompanying drawings and the description below. Other features, objects, and advantages of the inventions will be apparent from the description and drawings, and from the claims. All cited references, patents, and patent applications, inclusive of U.S. Application Ser. No. 60/484,327, filed on Jul. 2, 2003, and PCT/US03/15370 are incorporated by reference for all purposes.

### BRIEF DESCRIPTION OF THE DRAWINGS

FIG. 1 illustrates the location of a gene encoding SIRT1 and a linkage peak associated with AD.

### DETAILED DESCRIPTION

Alzheimer's Disease (AD) is genetically linked to a locus that includes the SIRT1 gene. This disclosure includes methods and compositions for evaluating genetic variances, e.g., within this locus, and for the association of these variances with AD. The disclosure also includes methods and compositions for modulating SIRT1 expression and/or activity. These methods and compositions can be used in the prevention or treatment of AD.

SIRT1

Late-onset Alzheimer's Disease has been linked to an 80 cM region close to D10S1225 on human chromosome 10q (Ertekin-Taner et al. Science 290:23032304 (2000); Myers et al. Science 290:2304-2305 (2000)). The SIRT1 gene has been mapped within this region (sequence tag site: stSG34970, International RH Mapping Consortium, Deloukas et al., Science, 282:744-746(1998); Schuler et al., Science 274:540-546 (1996)). Reported reference markers for the SIRT1 gene are D10S210-D10S537 (89.4-93.8 cM) and the physical position was mapped to 369.45 cR₃₀₀₀ (PO.94) (International RH Mapping Consortium Deloukas et al., Science, 282:744-746(1998); Schuler et al., Science 274:540-546 (1996)). The coding nucleotide sequence of human SIRT1 is shown in Table 1 (SEQ ID NO:1). A “SIRT1 locus” includes the genetic region between nucleotides 69527051 and 69606347 of chromosome 10, according to the numbering used by the Genbank Genome website June 2003. The term “SIRT1 gene” is used interchangeably with the term SIRT1 locus. An exemplary genomic sequence for this region is provided in the Appendix, as downloaded from a Homo sapiens chromosome 10 genomic contig NT_{—}008583.15 (>gi|29801680:18178747-18238747).

SIRT1 is a member of the Silent Information Regulator (SIR) family of genes. SIR proteins are involved in diverse processes from regulation of gene silencing to DNA repair and have been implicated in aging processes in yeast and worms. SIRT1 may regulate the susceptibility of neurons to AD-induced death, for example, by modulating apoptosis in neurons.

The proteins encoded by members of the SIR2 gene family show high sequence conservation in a 250 amino acid core domain. A well-characterized gene in this family is S. cerevisiae SIR2, which is involved in silencing HM loci that contain information specifying yeast mating type, telomere position effects and cell aging (Guarente, 1999; Kaeberlein et al., 1999; Shore, 2000). The yeast Sir2 protein belongs to a family of histone deacetylases (reviewed in Guarente, 2000; Shore, 2000). The Sir2 protein is a deacetylase which can use NAD as a cofactor (Imai et al., 2000; Moazed, 2001; Smith et al., 2000; Tanner et al., 2000; Tanny and Moazed, 2001). Unlike other deacetylases, many of which are involved in gene silencing, Sir2 is insensitive to histone deacetylase inhibitors like trichostatin A (TSA) (Imai et al., 2000; Landry et al., 2000a; Smith et al., 2000). Mammalian Sir2 homologs, such as SIRT1, have NAD-dependent deacetylase activity (Imai et al., 2000; Smith et al., 2000). Natural substrates for SIRT1 include histones and p53. SIRT1 can interact with a number of proteins which may also be substrates, e.g., p53, FOXO (e.g., FOXO4), re1A/p65, or bHLH repressors HES1 and HEY2.

SIRT1 proteins bind to a number of other proteins, referred to as “SIRT1 binding partners.” For example, SIRT1 binds to p53 and plays a role in the p53 pathway. SIRT1 proteins can also deacetylate histones. For example, SIRT1 can deacetylate lysines 9 or 14 of histone H3. Histone deacetylation alters local chromatin structure and consequently can regulate the transcription of a gene in that vicinity. Many of the SIRT1 binding partners are transcription factors, e.g., proteins that recognize specific DNA sites. Interaction between SIRT1 and SIRT1 binding partners can deliver SIRT1 to specific regions of a genome and can result in a local manifestation of substrates, e.g., histones and transcription factors localized to the specific region.

“SIRT1 proteins” and “SIRT1 polypeptides” are used interchangeably herein and refer to members of the Silent Information Regulator (SIR) 2 family of genes. In particular, the term “SIRT1 proteins” or “SIRT1 polypeptides” refers to a polypeptide that is at least 25% identical to the 250 amino acid conserved SIRT1 catalytic domain, amino acid residues 258 to 451 of SEQ ID NO: 2. SEQ ID NO:2 depicts the amino acid sequence of human SIRT1. In preferred embodiments, a SIRT1 polypeptide can be at least 30, 40, 50, 60, 70, 80, 85, 90, 95, 99% homologous to SEQ ID NO:2 or to the amino acid sequence between amino acid residues 258 and 451 of SEQ ID NO:2. In other embodiments, the SIRT1 polypeptide can be a fragment, e.g., a fragment of SIRT1 capable of one or more of: deacetylating a substrate in the presence of NAD and/or a NAD analog and capable of binding a target protein, e.g., a transcription factor. Such functions can be evaluated, e.g., by the methods described herein. In other embodiments, the SIRT1 polypeptide can be a “full length” SIRT1 polypeptide. The term “full length” as used herein refers to a polypeptide that has at least the length of a naturally-occurring SIRT1 polypeptide (or other protein described herein). A “full length” SIRT1 polypeptide or a fragment thereof can also include other sequences, e.g., a purification tag., or other attached compounds, e.g., an attached fluorophore, or cofactor. The term “SIRT1 polypeptides” can also include sequences or variants that include one or more substitutions, e.g., between one and ten substitutions, with respect to a naturally occurring Sir2 family member. A “SIRT1 activity” refers to one or more activity of SIRT1, e.g., deacetylation of a substrate (e.g., an amino acid, a peptide, or a protein), e.g., transcription factors (e.g., p53, FOXO, e.g., FOXO4) or histone proteins, (e.g., in the presence of a cofactor such as NAD and/or an NAD analog) and binding to a target, e.g., a target protein, e.g., a transcription factor.

Genetic Information

SIRT1 genetic information can be obtained, e.g., by evaluating genetic material (e.g., DNA or RNA) from a subject (e.g., as described below). Genetic information refers to any indication about nucleic acid sequence content at one or more nucleotides. Genetic information can include, for example, an indication about the presence or absence of a particular polymorphism, e.g., one or more nucleotide variations. Exemplary polymorphisms include a single nucleotide polymorphism (SNP), a restriction site or restriction fragment length, an insertion, an inversion, a deletion, a repeat (e.g., trinucleotide repeat, a retroviral repeat), and so forth.

Exemplary SIRT1 SNPs are listed in Table 1.

TABLE 1
Exemplary SIRT1 SNPs

| start | stop | dbSNP rs# | local loci | transID | avg. het | s.e. het |
|---|---|---|---|---|---|---|
| 69520160 | 69520160 | rs730821 | | | | 0 |
| 69520607 | 69520607 | rs3084650 | | | | 0 |
| 69530733 | 69530733 | rs4746715 | | | | 0 |
| 69531621 | 69531621 | rs4745944 | | | | 0 |
| 69535743 | 69535743 | rs3758391 | SIRT1: locus; | | 0.267438 | 0.153425 |
| 69536360 | 69536360 | rs3740051 | SIRT1: locus; | | 0.424806 | 0.114325 |
| 69536618 | 69536618 | rs932658 | SIRT1: locus; | | | 0 |
| 69536736 | 69536736 | rs3740053 | SIRT1: locus; | | | 0 |
| 69536742 | 69536742 | rs2394443 | SIRT1: locus; | | | 0 |
| 69539733 | 69539733 | rs932657 | SIRT1: intron; | | | 0 |
| 69540006 | 69540006 | rs737477 | SIRT1: intron; | | 0.118187 | 0.201473 |
| 69540390 | 69540390 | rs911738 | SIRT1: intron; | | | 0 |
| 69540762 | 69540762 | rs4351720 | SIRT1: intron; | | | 0 |
| 69540970 | 69540970 | rs2236318 | SIRT1: intron; | | 0.222189 | 0.135429 |
| 69541621 | 69541621 | rs2236319 | SIRT1: intron; | | 0.455538 | 0.102018 |
| 69544136 | 69544136 | rs768471 | SIRT1: intron; | | 0 | 0.01 |
| 69547213 | 69547213 | rs1885472 | SIRT1: intron; | | | 0 |
| 69549191 | 69549191 | rs2894057 | SIRT1: intron; | | | 0 |
| 69551326 | 69551326 | rs4746717 | SIRT1: intron; | | | 0 |
| 69557788 | 69557788 | rs2224573 | SIRT1: intron; | | | 0 |
| 69558999 | 69558999 | rs2273773 | SIRT1; | NM_012238; | 0.430062 | 0.135492 |
| 69559302 | 69559302 | rs3818292 | SIRT1: intron; | | 0.456782 | 0.10598 |
| 69564725 | 69564725 | rs1063111 | SIRT1; | NM_012238; | | 0 |
| 69564728 | 69564728 | rs1063112 | SIRT1; | NM_012238; | | 0 |
| 69564741 | 69564741 | rs1063113 | SIRT1; | NM_012238; | | 0 |
| 69564744 | 69564744 | rs1063114 | SIRT1; | NM_012238; | | 0 |
| 69565400 | 69565400 | rs3818291 | SIRT1: intron; | | 0.179039 | 0.132983 |
| 69566230 | 69566237 | rs5785840 | SIRT1: intron; | | | 0 |
| 69566318 | 69566318 | rs2394444 | SIRT1: intron; | | | 0 |
| 69567559 | 69567559 | rs1467568 | SIRT1: intron; | | | 0 |
| 69567728 | 69567728 | rs1966188 | SIRT1: intron; | | | 0 |
| 69568961 | 69568961 | rs2394445 | SIRT1; | NM_012238: UTR; | | 0 |
| 69568962 | 69568962 | rs2394446 | SIRT1; | NM_012238: UTR; | | 0 |
| 69569231 | 69569231 | rs4746720 | SIRT1; | NM_012238: UTR; | | 0 |
| 69569461 | 69569461 | rs752578 | SIRT1; | NM_012238: UTR; | | 0 |
| 69570479 | 69570479 | rs2234975 | SIRT1; | NM_012238: UTR; | | 0 |
| 69570580 | 69570580 | rs1022764 | SIRT1: locus; | | | 0 |
| 69570983 | 69570983 | rs1570290 | SIRT1: locus; | | 0.0392 | 0.167405 |
| 69572334 | 69572334 | rs2025162 | | | | 0 |
| 69573968 | 69573968 | rs4141919 | DKFZP564G092: locus; | | | 0 |
| 69574252 | 69574252 | rs14819 | DKFZP564G092: locus; | | | 0 |
| 69575032 | 69575032 | rs14840 | DKFZP564G092: locus; | | | |

It is possible to digitally record or communicate genetic information in a variety of ways. Typical representations include one or more bits, or a text string. For example, a biallelic marker can be described using two bits. In one embodiment, the first bit indicates whether the first allele (e.g., the minor allele) is present, and the second bit indicates whether the other allele (e.g., the major allele) is present. For markers that are multi-allelic, e.g., where greater than two alleles are possible, additional bits can be used as well as other forms of encoding (e.g., binary, hexadecimal text, e.g., ASCII or Unicode, and so forth). In some embodiments, the genetic information describes a haplotype, e.g., a plurality of polymorphisms on the same chromosome. However, in many embodiments, the genetic information is unphased.

Methods of Evaluating Genetic Material

There are numerous methods for evaluating genetic material to provide genetic information. These methods can be used to evaluate a SIRT1 locus as well as other loci.

Nucleic acid samples can analyzed using biophysical techniques (e.g., hybridization, electrophoresis, and so forth), sequencing, enzyme-based techniques, and combinations-thereof. For example, hybridization of sample nucleic acids to nucleic acid microarrays can be used to evaluate sequences in an mRNA population and to evaluate genetic polymorphisms. Other hybridization based techniques include sequence specific primer binding (e.g., PCR or LCR); Southern analysis of DNA, e.g., genomic DNA; Northern analysis of RNA, e.g., mRNA; fluorescent probe based techniques (see, e.g., Beaudet et al. (2001) Genome Res. 11(4):600-8); and allele specific amplification. Enzymatic techniques include restriction enzyme digestion; sequencing; and single base extension (SBE). These and other techniques are well known to those skilled in the art.

Electrophoretic techniques include capillary electrophoresis and Single-Strand Conformation Polymorphism (SSCP) detection (see, e.g., Myers et al. (1985) Nature 313:495-8 and Ganguly (2002) Hum Mutat. 19(4):334-42). Other biophysical methods include denaturing high pressure liquid chromatography (DHPLC).

In one embodiment, allele specific amplification technology that depends on selective PCR amplification may be used to obtain genetic information. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition, it is possible to introduce a restriction site in the region of the mutation to create cleavage-based detection (Gasparini et al. (1992) Mol. Cell Probes 6:1). In another embodiment, amplification can be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Enzymatic methods for detecting sequences include amplification based-methods such as the polymerase chain reaction (PCR; Saiki, et al. (1985) Science 230, 1350-1354) and ligase chain reaction (LCR; Wu. et al. (1989) Genomics 4, 560-569; Barringer et al. (1990), Gene 1989, 117-122; F. Barany. 1991, Proc. Natl. Acad. Sci. USA 1988, 189-193); transcription-based methods utilize RNA synthesis by RNA polymerases to amplify nucleic acid (U.S. Pat. No. 6,066,457; U.S. Pat. No. 6,132,997; U.S. Pat. No. 5,716,785; Sarkar et al., Science (1989) 244:331-34; Stofler et al., Science (1988) 239:491); NASBA (U.S. Pat. Nos. 5,130,238; 5,409,818; and 5,554,517); rolling circle amplification (RCA; U.S. Pat. Nos. 5,854,033 and 6,143,495) and strand displacement amplification (SDA; U.S. Pat. Nos. 5,455,166 and 5,624,825). Amplification methods can be used in combination with other techniques.

Other enzymatic techniques include sequencing using polymerases, e.g., DNA polymerases and variations thereof such as single base extension technology. See, e.g., U.S. Pat. No. 6,294,336; U.S. Pat. No. 6,013,431; and U.S. Pat. No. 5,952,174

Mass spectroscopy (e.g., MALDI-TOF mass spectroscopy) can be used to detect nucleic acid polymorphisms. In one embodiment, (e.g., the MassEXTEND™ assay, SEQUENOM, Inc.), selected nucleotide mixtures, missing at least one dNTP and including a single ddNTP is used to extend a primer that hybridizes near a polymorphism. The nucleotide mixture is selected so that the extension products between the different polymorphisms at the site create the greatest difference in molecular size. The extension reaction is placed on a plate for mass spectroscopy analysis.

Fluorescence based detection can also be used to detect nucleic acid polymorphisms. For example, different terminator ddNTPs can be labeled with different fluorescent dyes. A primer can be annealed near or immediately adjacent to a polymorphism, and the nucleotide at the polymorphic site can be detected by the type (e.g., “color”) of the fluorescent dye that is incorporated.

Hybridization to microarrays can also be used to detect polymorphisms, including SNPs. For example, a set of different oligonucleotides, with the polymorphic nucleotide at varying positions with the oligonucleotides can be positioned on a nucleic acid array. The extent of hybridization as a function of position and hybridization to oligonucleotides specific for the other allele can be used to determine whether a particular polymorphism is present. See, e.g., U.S. Pat. No. 6,066,454.

In one implementation, hybridization probes can include one or more additional mismatches to destabilize duplex formation and sensitize the assay. The mismatch may be directly adjacent to the query position, or within 10, 7, 5, 4, 3, or 2 nucleotides of the query position. Hybridization probes can also be selected to have a particular Tₘ, e.g., between 45-60° C., 55-65° C., or 60-75° C. In a multiplex assay, Tₘ's can be selected to be within 5, 3, or 2° C. of each other, e.g., probes for rs1800591and rs2866164 can be selected with these criteria.

It is also possible to directly sequence the nucleic acid for a particular genetic locus, e.g., by amplification and sequencing, or amplification, cloning and sequence. High throughput automated (e.g., capillary or microchip based) sequencing apparati can be used. In still other embodiments, the sequence of a protein of interest is analyzed to infer its genetic sequence. Methods of analyzing a protein sequence include protein sequencing, mass spectroscopy, sequence/epitope specific immunoglobulins, and protease digestion.

Any combination of the above methods can also be used. The above methods can be used to evaluate any genetic locus, e.g., in a method for analyzing genetic information from particular groups of individuals or in a method for analyzing a polymorphism associated with AD, e.g., the SIRT1 locus.

Evaluating Markers of AD

A variety of criteria, including genetic, biochemical, physiological, and cognitive criteria, can be used to evaluate AD in a subject. Symptoms and diagnosis of AD are known to medical practitioners. Some exemplary symptoms and markers of AD are presented below. Information about these indications and other indications known to be associated with AD can be used as an “AD-related parameter.” An AD-related parameter can include qualitative or quantitative information. An example of quantitative information is a numerical value of one or more dimensions, e.g., a concentration of a protein or a tomographic map. Qualitative information can include an assessment, e.g., a physician's comments or a binary (“yes”/“no”) and so forth. An AD-related parameter includes information that indicates that the subject is not diagnosed with AD or does not have a particular indication of AD, e.g., a cognitive test result that is not typical of AD or a genetic APOE polymorphism not associated with AD.

Progressive cognitive impairment is a hallmark of AD. This impairment can present as decline in memory, judgment, decision making, orientation to physical surroundings, and language (Nussbaum and Ellis, New Eng. J. Med. 348(14):1356-1364 (2003)). Exclusion of other forms of dementia can assist in making a diagnosis of AD.

Neuronal death leads to progressive cerebral atrophy in AD patients. Inaging techniques (e.g., magnetic resonance imaging, or computed tomography) can be used to detect AD-associated lesions in the brain and/or brain atrophy.

AD patients may exhibit biochemical abnormalities that result from the pathology of the disease. For example, levels of tau protein in the cerebrospinal fluid is elevated in AD patients (Andreasen, N. et al. Arch Neurol. 58:349-356 (2001)). Levels of amyloid beta 42 (Aβ42) peptide can be reduced in CSF of AD patients (Galasko, D., et al. Arch. Neurol. 55:937-945 (1998)). Levels of Aβ42 can be increased in the plasma of AD patients (Ertekein-Taner, N., et al. Science 290:2303-2304 (2000)). Techniques to detect biochemical abnormalities in a sample from a subject include cellular, immunological, and other biological methods known in the art. For general guidance, see, e.g., techniques described in Sambrook & Russell, Molecular Cloning: A Laboratory Manual, 3^{rd} Edition, Cold Spring Harbor Laboratory, N.Y. (2001), Ausubel et al., Current Protocols in Molecular Biology (Greene Publishing Associates and Wiley Interscience, N.Y. (1989), (Harlow, E. and Lane, D. (1988) Antibodies: A Laboratory Manual, Cold Spring Harbor Laboratory Press, Cold Spring Harbor, N.Y., and updated editions thereof.

For example, antibodies, other immunoglobulins, and other specific binding ligands can be used to detect a biomolecule, e.g., a protein or other antigen associated with AD. For example, one or more specific antibodies can be used to probe a sample. Various formats are possible, e.g., ELISAs, fluorescence-based assays, Western blots, and protein arrays. Methods of producing polypeptide arrays are described in the art, e.g., in De Wildt et al. (2000). Nature Biotech. 18, 989-994; Lueking et al. (1999). Anal. Biochem. 270, 103-111; Ge, H. (2000). Nucleic Acids Res. 28, e3, I-VII; MacBeath, G., and Schreiber, S. L. (2000). Science 289, 1760-1763; and WO 99/51773A1.

Proteins can also be analyzed using mass spectroscopy, chromatography, electrophoresis, enzyme interaction or using probes that detect post-translational modification (e.g., a phosphorylation, ubiquitination, glycosylation, methylation, or acetylation).

Nucleic acid expression can be detected in cells from a subject, e.g., removed by surgery, extraction, post-mortem or other sampling (e.g., blood, CSF). Expression of one or more genes can be evaluated, e.g., by hybridization based techniques, e.g., Northern analysis, RT-PCR, SAGE, and nucleic acid arrays. Nucleic acid arrays are useful for profiling multiple mRNA species in a sample. A nucleic acid array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

Metabolites that are associated with AD can be detected by a variety of means, including enzyme-coupled assays, using labeled precursors, and nuclear magnetic resonance (NMR). For example, NMR can be used to determine the relative concentrations of phosphate-based compounds in a sample, e.g., creatine levels. Other metabolic parameters such as redox state, ion concentration (e.g., Ca²⁺)(e.g., using ion-sensitive dyes), and membrane potential can also be detected (e.g., using patch-clamp technology).

Information about an AD-associated marker can be recorded and/or stored in a computer-readable format. Typically the information is linked to a reference about the subject and also is associated (directly or indirectly) with information about the identity of one or more nucleotides in the subject's SIRT1 genes.

Identifying Relevant Genotypes

Methods for identifying genotypes associated with AD can include comparisons to one or more reference sequences or an association study among individuals that have a particular characteristic, e.g., a particular AD-associated parameter or an AD diagnosis.

Multiple sets of reference sequences may be used for comparison. Exemplary reference sequences include sequences from subjects at risk for or diagnosed with AD and sequences from subjects that are not at risk for or diagnosed with AD. In some embodiments, reference subjects include long-lived individuals (LLI's), e.g., nonagenarians and centenarians. Such long-lived individuals may be cognitively intact at an old age (e.g., after attaining an age of 85, 90, 95, 97, 98, 99, 100, or 105). Such long-lived individuals may be also be free of one or more symptoms associated with AD, e.g., they may not have been diagnosed with AD. As described below, LLI's are a particular useful set of reference subjects since their survival to an old age can at least in some part be attributed to their genotype, e.g., they are likely to be free of alleles causative of a fatal disease at an earlier age.

There are many ways of selecting a subset of reference subjects from a set of potential subjects, particular where stratification of the sampled group may be an issue. U.S. patent application Ser. No. 10/378,397, filed Mar. 3, 2003, describes, inter alia, exemplary methods for reducing stratification, including a method that minimizes a multivariate distance between a group of subjects and a group of references.

The methods described herein can be used to evaluate genes and genetic traits that may be associated with a state such as AD, but also—by extension—other states, e.g., a human disease (other than AD) and a phenotype, such as resistance to an environmental condition, a physical manifestation, and a behavior. In just one application, the method is used to evaluate genes that affect lifespan regulation or an age-related disease or predisposition to such a disease. In one embodiment, the disease is Alzheimer's Disease (AD). Other exemplary age-related diseases include: cancer (e.g., breast cancer, colorectal cancer, CCL, CML, prostate cancer), neurodegenerative diseases (e.g., Huntington's disease ALS, Parkinson's disease, Amyotrophic Lateral Sclerosis, Multiple Sclerosis, and disorders caused by polyglutamine aggregation), skeletal muscle atrophy; adult-onset diabetes, diabetic nephropathy, neuropathy (e.g., sensory neuropathy, autonomic neuropathy, motor neuropathy, retinopathy); obesity; bone resorption, age-related macular degeneration, AIDS related dementia, ALS, Bell's Palsy, atherosclerosis, cardiac diseases (e.g., cardiac dysrhymias, chronic congestive heart failure, ischemic stroke, coronary artery disease and cardiomyopathy), chronic renal failure, type 2 diabetes, ulceration, cataract, presbiopia, glomerulonephritis, Guillan-Barre syndrome, hemorrhagic stroke, rheumatoid arthritis, inflammatory bowel disease, SLE, Crohn's disease, osteoarthritis, osteoporosis, pneumonia, and urinary incontinence. Symptoms and diagnosis of such diseases are well known to medical practitioners.

By evaluating one or more genetic loci, it is possible to determine an association for each locus or for each allele of each locus, and a phenotype. One type of test of association is the G-Test, but other statistical measures can also be used. A high degree of association, e.g., a high chi-square statistic, can indicate that a particular locus is associated with a state (e.g., a phenotype). This type of associational study can be used to map a genetic locus that is associated with the state. Associated loci can be used, e.g., for diagnostic evaluations (e.g., genetic counseling, risk evaluation, prophylactic care, care management, and so forth) and for research (e.g., identifying targets for therapeutics).

As seen herein, it is also possible to identify genes associated with disorders by using a method that includes: a) identifying a plurality of human individuals characterized by a disorder or having a genetic relationship with an subject characterized by the disorder; and b) comparing distribution of a plurality of genetic markers among the subjects of the first plurality to distribution of markers of the plurality of genetic markers among subjects of a second plurality of human subjects, wherein the human subjects of the second plurality have attained at least 90, 95, 98, or 100 years of age. For example, the plurality of genetic markers includes at least one, 10, 20, 30 or 50 markers from each chromosome. In one embodiment, the plurality of genetic markers includes at least one marker from chromosome X (e.g., the AD6 locus, e.g., the 17 million base pair region of human chromosome 10 or at least one marker in the SIRT1 gene). The method can further include evaluating a measure of linkage disequilibrium (e.g., a LOD score). For example, each subject of the first plurality is suffering or at risk for an age-associated disorder or each subject of the first plurality is genetically related to an subject suffering or at risk for an age-associated disorder.

In one embodiment, the age-associated disorder is one of the following disorders: cancer (e.g., breast cancer, colorectal cancer, CCL, CML, prostate cancer); skeletal muscle atrophy; adult-onset diabetes; diabetic nephropathy, neuropathy (e.g., sensory neuropathy, autonomic neuropathy, motor neuropathy, retinopathy); obesity; bone resorption; age-related macular degeneration, ALS, Bell's Palsy, atherosclerosis, cardiac diseases (e.g., cardiac dysrhythmias, chronic congestive heart failure, ischernic stroke, coronary artery disease and cardiomyopathy), chronic renal failure, type 2 diabetes, ulceration, cataract, presbiopia, glomerulonephritis, Guillan-Barre syndrome, hemorrhagic stroke, rheumatoid arthritis, inflammatory bowel disease, multiple sclerosis, SLE, Crohn's disease, osteoarthritis, Parkinson's disease, pneumonia, and urinary incontinence. In one embodiment, the age-associated disorder is Alzheimer's disease.

In one embodiment, the first plurality includes at least 50, 100, 150, 200, or 300 subjects. In one embodiment, the human subjects of the second plurality are free of an AD diagnosis. For example, the human subjects of the second plurality are cognitively intact at the age of 85, 90, 95, 98, or 100 and/or the human subjects of the second plurality are free of a symptom or diagnosis of the disorder. In one embodiment, the second plurality includes at least 50, 100, 150, 200, 300, 500 or 800 subjects.

Computer Implementations

Certain implementations include digital electronic circuitry, or in computer hardware, firmware, software, or in combinations thereof. Methods can be implemented using a computer program product tangibly embodied in a machine-readable storage device for execution by a programmable processor; and method actions can be performed by a programmable processor executing a program of instructions to perform functions by operating on input data and generating output. For example, many methods can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object oriented programming language, or in assembly or machine language if desired; and in any case, the language can be a compiled or interpreted language. Suitable processors include, by way of example, both general and special purpose microprocessors. A processor can receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including, by way of example, semiconductor memory devices, such as EPROM, EEPROM, and flash memory devices; magnetic disks such as, internal hard disks. and removable disks; magneto-optical disks; and CD_ROM disks. Any of the foregoing can be supplemented by, or incorporated in, ASICs (application-specific integrated circuits).

In one implementation, information about a set of potential reference sequences and/or reference subjects (e.g., long-lived individuals) is stored on a server. A user can send information about case groups to the server, e.g., from a remote computer that communicates with the server using a network, e.g., the Internet. The case groups can be, e.g., individuals associated with an age-related disorder. The server can compare the information about the test sequences and/or test subjects and select a subset of members from the potential controls, e.g., to minimize a distance measure that is a function of the case groups and the selected subset. The server can return information about the subset (e.g., identifiers or other data) to the user or can return an evaluation that compares a feature of the case group to the members of the selected subset (e.g., a statistical score that evaluates probability of association with the case group relative to the selected subset). Accordingly, the server can include a electronic interface for receiving information from a user or from an apparatus that provides information about a biological property and software configured to execute identify a subset of data objects using a comparison described herein.

In another implementation, information about a subject's SIRT1 locus (e.g., information about one or both SIRT1 alleles) is stored on a server. A user can send information about the subject (e.g., a patient, a relative of a patient, a sample of gametes (e.g., sperm or oocytes), fetal cells, or a candidate for a treatment) to the server, e.g., from a remote computer that communicates with the server using a network, e.g., the Internet. The server can compare the information about the subject, e.g., to reference information to produce an indication as to the individual propensity for AD. For example, the reference information can be information derived from a reference individual, a particular sequence, or a population of sequences. The indication can be, for example, qualitative or quantitative. An exemplary qualitative indication includes a binary output or a descriptive output (e.g., text or other symbols indicating degree of propensity for AD). An exemplary qualitative indication includes a statistical measure of the probability of developing AD, a score, a percentage, or a parameter for a risk evaluation (e.g., a parameter that can be used in a financial evaluation).

It is also possible for the server to return the indication or information about related subjects (e.g., family members or subjects with similar SIRT1 loci), e.g., to the user. For example, the server can build a family tree based on a set of related subject. Each individual can be, e.g., assigned a statistical score that evaluates probability of AD as a function of an AD-associated gene locus, e.g., the SIRT1 locus, and/or other factors. Accordingly, the server can include an electronic interface for receiving information from a user or from an apparatus that provides information about an AD-associated gene locus.

In one method, information about the subject's SIRT1 locus, e.g., the result of evaluating a polymorphism of a locus described herein, is provided (e.g., communicated, e.g., electronically communicated) to a third party, e.g., a hospital, clinic, a government entity, reimbursing party or insurance company (e.g., a life insurance company). For example, choice of medical procedure, payment for a medical procedure, payment by a reimbursing party, or cost for a service or insurance can be function of the information.

In one embodiment, a premium for insurance (e.g., life or medical) is evaluated as a function of information about one or more longevity associated polymorphisms, e.g., a polymorphism described herein, e.g., an SIRT1 gene polymorphism. For example, premiums can be increased (e.g., by a certain percentage) if a first polymorphism is present in the candidate insured, or decreased if a second polymorphism is present. Premiums can also be scaled depending on heterozygosity or homozygosity. For example, premiums can be assessed to distribute risk, e.g., commensurate with the allele distribution for the particular polymorphism. In another examples, premiums are assessed as a function of actuarial data that is obtained from individuals with one or more AD-associated polymorphisms.

Genetic information about one or more AD-associated polymorphisms, e.g., a polymorphism described herein, e.g., a SIRT1 gene polymorphism, can be used, e.g., in an underwriting process for life insurance. The information can be incorporated into a profile about a subject. Other information in the profile can include, for example, date of birth, gender, marital status, banking information, credit information, children and so forth. An insurance policy can be recommended as a function of the genetic information along with one or more other items of information in the profile. An insurance premium or risk assessment can also be evaluated as function of the genetic information. In one implementation, points are assigned for presence or absence of a particular allele. The total points for AD polymorphisms and other risk parameters are summed. A premium is calculated as a function of the points, and optionally one or more other parameters.

In one embodiment, information about an AD-associated polymorphism, e.g., a polymorphism described herein is analyzed by a function that determines whether to authorize or transfer of funds to pay for a service or treatment provided to a subject. For example, an allele that is not associated with AD can trigger an outcome that indicates or causes a refusal to pay for a service or treatment provided to a subject. For example, an entity, e.g., a hospital, care giver, governnent entity, or an insurance company or other entity which pays for, or reimburses medical expenses, can use the outcome of a method described herein to determine whether a party, e.g., a party other than the subject patient, will pay for services or treatment provided to the patient. For example, a first entity, e.g., an insurance company, can use the outcome of a method described herein to determine whether to provide financial payment to, or on behalf of, a patient, e.g., whether to reimburse a third party, e.g., a vendor of goods or services, a hospital, physician, or other care-giver, for a service or treatment provided to a patient. For example, a first entity, e.g., an insurance company, can use the outcome of a method described herein to determine whether to continue, discontinue, enroll an individual in an insurance plan or program, e.g., a health insurance or life insurance plan or program.

Pharmacogenomics

Both prophylactic and therapeutic methods of treatment may be specifically tailored or modified, based on knowledge obtained from a pharmacogenomics analysis. In particular, a subject can be treated based on the presence or absence of a genetic polymorphism associated with AD, e.g., a polymorphism associated with the SIRT1 locus. Pharmacogenomics allows a clinician or physician to target prophylactic or therapeutic treatments to patients who will most benefit from the treatment and to avoid the treatment of patients who will experience toxic or other undesirable drug-related side effects. In particular, a diet or drug that affects AD can be prescribed as a function of the subject's SIRT1 locus. For example, if the individual's SIRT1 locus includes an allele that is predisposed to AD relative to other alleles, the individual can be indicated for a prophylactic treatment for a drug that alleviates AD. In another example, the individual is placed in a monitoring program, e.g., to closely monitor for physical manifestations of AD onset.

Screening Assays

The invention includes methods of screening for compounds that modulate SIRT1 activity. Such compounds include a compound which directly interacts with SIRT1 and compounds which alter SIRT1 protein or RNA expression. Such compounds can be identified as candidates for the prevention or treatment of AD.

One method can include providing a compound which interacts with SIRT1 and evaluating the effect of the compound on a biochemical, cellular, or organismal phenotype associated with AD, e.g., as described herein. Another method can include screening for compounds using a method that includes evaluating the compounds for modulation of SIRT1 activity and evaluating the effect of the compound on a biochemical, cellular, or organismal phenotype associated with AD, e.g., as described herein. The evaluations can be performed in either order. For example, a library of compounds can be vetted using the first criterion (modulation of SIRT1 activity) to provide a smaller set of compounds, and then evaluating compounds from the smaller set for an effect on an AD phenotype. The vetting can also be done in the opposite order.

Compounds which interact with SIRT1 can be identified, e.g., by in vitro or in vivo assays. Exemplary in vitro assays for SIRT1 activity include cell free assays, e.g., assays in which an isolated SIRT1 polypeptide (including a polypeptide that includes a fragment of at least 100 amino acids of SIRT1, e.g., a fragment described herein) is contacted with a test compound.

When both the assay for screening a compound for the ability to interact with SIRT1 and the assay for determining effect on SIRT1 are performed in vivo, e.g., in cell based assays, the assays can be performed in the same or different cells. For example, one or both of the assays can be performed in tissue culture (e.g., PC12 cells, or primary neuronal cultures) or in an organism (e.g., a mammal, e.g., a human).

In preferred embodiments, the assays are performed in the presence of a SIRT1 cofactor such as NAD and/or NAD analogs. In some embodiments, the co-factor is added to the cell culture or in vitro assay, e.g., the NAD and/or an NAD analog can be placed in sufficient proximity to cause a SIRT1 activity such as deacetylation. “NAD” refers to nicotinamide adenine dinucleotide. An “NAD analog” as used herein refers to a compound (e.g., a synthetic or naturally occurring chemical, drug, protein, peptide, small organic molecule) which possesses structural similarity to component groups of NAD (e.g., adenine, ribose and phosphate groups) or functional similarity (e.g., deacetylates p53 in the presence of SIRT1). For example, an NAD analog can be 3-aminobenzamide or 1,3-dihydroisoquinoline (H. Vaziri et al., EMBO J. 16:6018-6033 (1997)).

Described below are exemplary methods for identifying compounds that interact with SIRT1 and can modulate SIRT1 activity or expression. Preferably, compounds can be identified which interact with, e.g., bind to, SIRT1 and increase at least one SIRT1 activity, e.g., deacetylation. Deacetylation of a substrate by SIRT1 has been found to decrease substrate-induced apoptosis, e.g., caspase-2 induced apoptosis. In many instances, such acetylated substrates may play a role in apoptosis of stressed and/or damaged cells, e.g., cells exposed to aggregated a β-amyloid (Aβ). Thus, in some embodiments, it is desirable to identify compounds which interact with SIRT1 and increase SIRT1 expression and/or a SIRT1 activity, thereby decreasing apoptosis in a cell, e.g., a neuronal cell.

The phrase “deacetylating a substrate” or “deacetylating a transcription factor” refers to the removal of one or more acetyl groups (e.g., CH₃CO²⁻) from the substrate or transcription factor that is acetylated on at least one amino acid residue. The substrate can be a single amino acid (e.g., an acetylated lysine), a peptide (e.g., a N-terminal peptide of a histone, or an acetylated p53 peptide), or a protein. An acetylated substrate can include a fluorophore, e.g., which can be used to monitor the acetylation states of the substrate. The “Fleur-de-Lys™” substrate from BIOMOL® includes one such exemplary modification. “Acetylation status” refers to the presence or absence of one or more acetyl groups (e.g. CH₃CO²⁻) at one or more lysine (K) residues of a substrate, e.g., a transcription factor. For example, the presence of an acetylate groups can be found at one or more of: K370, K371, K372, K381, and/or K382 of the p53 sequence. “Altering the acetylation status” refers to adding or removing one or more acetyl groups (e.g., CH₃CO²⁻). For example, adding or removing one or more acetyl groups of p53 at one or more lysine (K) residues, e.g., K370, K371, K372, K381, and/or K382.

A variety of techniques may be utilized to modulate the expression, synthesis, or activity of such target genes and/or proteins. Such molecules may include, but are not limited to small organic molecules, peptides, antibodies, nucleic acids, antisense nucleic acids, RNAi, ribozyme molecules, triple helix molecules, and the like.

The following assays provide methods (also referred to herein as “evaluating a compound” or “screening a compound”) for identifying modulators, i.e., candidate or test compounds (e.g., peptides, peptidomimetics, small molecules or other drugs) which interact with and/or modulate SIRT1 activity, e.g., have a stimulatory or inhibitory effect on, for example, SIRT1 expression and/or activity, or have a stimulatory or inhibitory effect on, for example, the expression or activity of a SIRT1 substrate. Such compounds can be agonists or antagonists of SIRT1. In preferred embodiments, the screening assays described herein are used to identify candidates which function as SIRT1 agonists. As described herein, such a SIRT1 agonist can decrease apoptosis of a cell, which has practical utility, e.g., in AD prevention or treatment. Some of these assays may be performed in animals, e.g., mammals, in organs, in cells. Others may be performed in animals, e.g., mammals, in organs, in cells, in cell extracts, e.g., purified or unpurified nuclear extracts, intracellular extracts, in purified preparations, in cell-free systems, in cell fractions enriched for certain components, e.g., organelles or compounds, or in other systems known in the art. Given the teachings herein and the state of the art, a person of ordinary skill in the art would be able to choose an appropriate system and assay for practicing the methods of the present invention.

Some exemplary screening assays for assessing activity or function include one or more of the following features:
use of a transgenic cell, e.g., with a transgene encoding SIRT1 or a mutant thereof;
use of a mammalian cell that expresses SIRT1;
detection of binding of a labeled compound to SIRT1 or a SIRT1 binding partner where the compound is, for example, a peptide, protein, antibody or small organic molecule; e.g., the compound stimulates an interaction between SIRT1 and a SIRT1 binding partner
use of assays that detect interaction between SIRT1 and a SIRT1 binding partner, e.g., a transcription factor (e.g., p53 or FOXO, e.g., FOXO4, re1A/p65, or bHLH repressors HES1 and HEY2), or fragments thereof, for example, a proximity assay, e.g., a fluorescence proximity assays
use of radio-labelled substrates, e.g. ³⁵S, ³H, ¹⁴C, e.g., to determine acetylation status, metabolic status, rate of protein synthesis, inter alia.
use of antibodies specific for certain acetylated or de-acetylated forms of the substrate.

Various screening assays are described in more detail below.

A “compound” or “test compound” can be any chemical compound, for example, a macromolecule (e.g., a polypeptide, a protein complex, or a nucleic acid) or a small molecule (e.g., an amino acid, a nucleotide, an organic or inorganic compound). The test compound can have a formula weight of less than about 10,000 grams per mole, less than 5,000 grams per mole, less than 1,000 grams per mole, or less than about 500 grams per mole. The test compound can be naturally occurring (e.g., a herb or a nature product), synthetic, or both. Examples of macromolecules are proteins, protein complexes, and glycoproteins, nucleic acids, e.g., DNA, RNA (e.g., double stranded RNA or RNAi) and PNA (peptide nucleic acid). Examples of small molecules are peptides, peptidomimetics (e.g., peptoids), amino acids, amino acid analogs, polynucleotides, polynucleotide analogs, nucleotides, nucleotide analogs, organic or inorganic compounds e.g., heteroorganic or organometallic compounds. One exemplary type of protein compound is an antibody or a modified scaffold domain protein. A test compound can be the only substance assayed by the method described herein. Alternatively, a collection of test compounds can be assayed either consecutively or concurrently by the methods described herein.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like). Additional examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et aL. (1994)Angew. Chem. Int. Ed. Engl. 33:2059; Carell et al. (1994) Angew. Chem. Int. Ed. Engl. 33:2061; and Gallop et al. (1994) J. Med. Chem. 37:1233.

Some exemplary libraries are used to generate variants from a particular lead compound. One method includes generating a combinatorial library in which one or more functional groups of the lead compound are varied, e.g., by derivatization. Thus, the combinatorial library can include a class of compounds which have a common structural feature (e.g., framework).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

The test compounds of the present invention can also be obtained from: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological libraries include libraries of nucleic acids and libraries of proteins. Some nucleic acid libraries encode a diverse set of proteins (e.g., natural and artificial proteins; others provide, for example, functional RNA and DNA molecules such as nucleic acid aptamers or ribozymes. A peptoid library can be made to include structures similar to a peptide library. (See also Lam (1997) Anticancer Drug Des. 12:145). A library of proteins may be produced by an expression library or a display library (e.g., a phage display library).

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In Vitro Assays

Exemplary in vitro assays include assays for a binding interaction or a catalytic activity, e.g., a deacetylase activity. Deacetylase assays include those described above and in US 2003-0082668. In some embodiments, interaction with, e.g., binding of, SIRT1 can be assayed in vitro. The reaction mixture can include a SIRT1 co-factor such as NAD and/or a NAD analog.

In other embodiments, the reaction mixture can include a SIRT1 binding partner, e.g., a transcription factor, e.g., a transcription and compounds can be screened, e.g., in an in vitro assay, to evaluate the ability of a test compound to modulate interaction between SIRT1 and a SIRT1 binding partner. This type of assay can be accomplished, for example, by coupling one of the components, with a radioisotope or enzymatic label such that binding of the labeled component to the other can be determined by detecting the labeled compound in a complex. A component can be labeled with ¹²⁵I, ³⁵S, ¹⁴C, or ³H, either directly or indirectly, and the radioisotope detected by direct counting of radioemmission or by scintillation counting. Alternatively, a component can be enzymatically labeled with, for example, horseradish peroxidase, alkaline phosphatase, or luciferase, and the enzymatic label detected by determination of conversion of an appropriate substrate to product. Competition assays can also be used to evaluate a physical interaction between a test compound and a target.

Cell-free assays involve preparing a reaction mixture of the target protein (e.g., SIRT1) and the test compound under conditions and for a time sufficient to allow the two components to interact and bind, thus forming a complex that can be removed and/or detected.

The interaction between two molecules can also be detected, e.g., using a fluorescence assay in which at least one molecule is fluorescently labeled. One example of such an assay includes fluorescence energy transfer (FET or FRET for fluorescence resonance energy transfer) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FET binding event can be conveniently measured through standard fluorometric detection means well known in the art (e.g., using a fluorimeter).

Another example of a fluorescence assay is fluorescence polarization (FP). For FP, only one component needs to be labeled. A binding interaction is detected by a change in molecular size of the labeled component. The size change alters the tumbling rate of the component in solution and is detected as a change in FP. See, e.g., Nasir et al. (1999) Comb Chem HTS 2:177-190; Jameson et al. (1995) Methods Enzymol 246:283; Seethala et al. (1998) Anal Biochem. 255:257. Fluorescence polarization can be monitored in multiwell plates, e.g., using the Tecan Polarion™ reader. See, e.g., Parker et al. (2000) Journal of Biomolecular Screening 5:77 -88; and Shoeman, et al.. (1999) 38, 16802-16809.

In another embodiment, determining the ability of the SIRT1 protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, SIRT1 is anchored onto a solid phase. The SIRT1/test compound complexes anchored on the solid phase can be detected at the end of the reaction, e.g., the binding reaction. For example, SIRT1 can be anchored onto a solid surface, and the test compound, (which is not anchored), can be labeled, either directly or indirectly, with detectable labels discussed herein.

It may be desirable to immobilize either the SIRT1 or an anti-SIRT1 antibody to facilitate separation of complexed from uncomplexed forms of one or both of the proteins, as well as to accommodate automation of the assay. Binding of a test compound to a SIRT1 protein, or interaction of a SIRT1 protein with a second component in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/SIRT1 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or SIRT1 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of SIRT1 binding or activity determined using standard techniques.

Other techniques for immobilizing either a SIRT1 protein or a target molecule on matrices include using conjugation of biotin and streptavidin. Biotinylated SIRT1 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface, e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with a SIRT1 protein or target molecules but which do not interfere with binding of the SIRT1 protein to its target molecule. Such antibodies can be derivatized to the wells of the plate, and unbound target or the SIRT1 protein trapped in the wells by antibody conjugation. Methods for detecting such complexes, in addition to those described above for the GST-immobilized complexes, include immunodetection of complexes using antibodies reactive with the SIRT1 protein or target molecule, as well as enzyme-linked assays which rely on detecting an enzymatic activity associated with the SIRT1 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the SIRT1 protein or biologically active portion thereof with a known compound which binds a SIRT1 to form an assay mixture, contacting the assay mixture with a test compound, and determining the ability of the test compound to interact with a SIRT1 protein, wherein determining the ability of the test compound to interact with the SIRT1 protein includes determining the ability of the test compound to preferentially bind to the SIRT1 or biologically active portion thereof, or to modulate the activity of a target molecule, as compared to the known compound.

The target products can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target product. Such compounds can include, but are not limited to molecules such as antibodies, peptides, and small molecules. The preferred targets/products for use in this embodiment include the SIRT1 binding partners.

To identify compounds that interfere with the interaction between the target product and its binding partner(s), a reaction mixture containing the target product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target and its cellular or extracellular binding partner. Control reaction mixtures are incubated without the test compound or with a placebo. The formation of any complexes between the target product and the cellular or extracellular binding partner is then detected. The formation of a complex in the control reaction, but not in the reaction mixture containing the test compound, indicates that the compound interferes with the interaction of the target product and the interactive binding partner. Additionally, complex formation within reaction mixtures containing the test compound and normal target product can also be compared to complex formation within reaction mixtures containing the test compound and mutant target product. This comparison can be important in those cases wherein it is desirable to identify compounds that disrupt interactions of mutant but not normal target products.

These assays can be conducted in a heterogeneous or homogeneous format. Heterogeneous assays involve anchoring either the target product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target product or the partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment, a homogeneous assay can be used. For example, a preformed complex of the target product and the interactive cellular or extracellular binding partner product is prepared in that either the target products or their binding partners are labeled, but the signal generated by the label is quenched due to complex formation (see, e.g., U.S. Pat. No. 4,109,496 that utilizes this approach for inirnmunoassays). The addition of a test substance that competes with and displaces one of the species from the preformed complex will result in the generation of a signal above background. In this way, test substances that disrupt target product-binding partner interaction can be identified.

In yet another aspect, the SIRT1 proteins can be used as “bait proteins” in a two-hybrid assay or three-hybrid assay (see, e.g., U.S. Pat. No. 5,283,317; Zervos et al. (1993) Cell 20 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with SIRT1 (“SIRT1-binding proteins”) and are involved in SIRT1 activity. Such SIRT1 binding partners can be activators or inhibitors of signals by the SIRT1 proteins.

The two-hybrid system is based on the modular nature of most transcription factors, which consist of separable DNA-binding and activation domains. Briefly, the assay utilizes two different DNA constructs. In one construct, the gene that codes for a SIRT1 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the SIRT1 protein can be the fused to the activator domain.) If the “bait” and the “prey” proteins are able to interact, in vivo, forming a SIRT1-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the SIRT1 protein. In another embodiment, the two-hybrid assay is used to monitor an interaction between two components, e.g., SIRT1 and, e.g., p53, that are known to interact. The two hybrid assay is conducted in the presence of a test compound, and the assay is used to determine whether the test compound enhances or diminishes the interaction between the components.

In another embodiment, modulators of SIRT1 gene expression are identified. For example, a cell or cell free mixture is contacted with a candidate compound and the expression of the SIRT1 mRNA or protein evaluated relative to the level of expression of SIRT1 mRNA or protein in the absence of the candidate compound. When expression of the SIRT1 mRNA or protein is greater in the presence of the candidate compound than in its absence, the candidate compound is identified as a stimulator of SIRT1 mRNA or protein expression. Alternatively, when expression of the SIRT1 mRNA or protein is less (statistically significantly less) in the presence of the candidate compound than in its absence, the candidate compound is identified as an inhibitor of the SIRT1 mRNA or protein expression. The level of the SIRT1 mRNA or protein expression can be determined by methods for detecting SIRT1 mRNA or protein, e.g., using probes or antibodies, e.g., labelled probes or antibodies.

Cell-Based Assays

Cell-based assays can be used to evaluate SIRT1 activity in a cell and also as a cell-based method to evaluate a compound for an effect on AD. Useful assays include assays in which apoptosis is measured and/or assays in which cellular responses to amyloid peptides are measured. See, for example, Troy, C. et al. J. Neurosci. 20(4):1386-1392 (2000).

An exemplary cell based assay can include contacting a cell expressing SIRT1 with a test compound and determining the ability of the test compound to modulate (e.g. stimulate or. inhibit) an activity of SIRT1, and/or determine the ability of the test compound to modulate SIRT1 expression, e.g., by detecting SIRT1 nucleic acids (e.g., mRNA or cDNA) or proteins in the cell. A preferred activity is the deacetylation function of SIRT1 of transcription factors; a further preferred activity is the ability to modulate apoptosis. Determining the ability of the test compound to modulate the activity of SIRT1 can be accomplished, for example, by determining the ability of SIRT1 to bind to or interact with the test molecule, and by determining the ability of the test molecule to modulate apoptosis. This assay can be used, e.g., to identify compounds that increase SIRT expression and/or activity and/or effect, e.g., decrease apoptosis or to identify compounds that decrease SIRT expression and/or activity and/or effect, e.g., enhance apoptosis. Such cells can be recombinant or non-recombinant, such as cell lines that express the SIRT1 gene.

In some embodiments, the cells can be recombinant or non-recombinant cells which express a SIRT1 binding partner or substrate (natural or artificial). Preferred systems include mammalian or yeast cells that express SIRT1. In utilizing such systems, cells are exposed to compounds suspected of increasing SIRT expression and/or SIRT1 activity. After exposure, the cells are assayed, for example, for expression of the SIRT1 gene or activity of the SIRT1 protein. Alternatively, the cells may also be assayed for the activation or inhibition of the deacetylation function of SIRT1, or the apoptotic or cytostatic function. In one embodiment, the visual assessment can be used for evidence of apoptosis, e.g., nuclear fragmentation.

Another preferred cell for a cell-based assay comprises a yeast cell transformed with a vector comprising the Sir2 gene, a homolog of human SIRT1.

A cell used in the methods described herein can be from a stable cell line or a primary culture obtained from an organism, e.g., a organism treated with the test compound.

In addition to cell-based and in vitro assay systems, non-human organisms, e.g., transgenic non-human organisms, can also be used. A transgenic organism is one in which a heterologous DNA sequence is chromosomally integrated into the germ cells of the animal. A transgenic organism will also have the transgene integrated into the chromosomes of its somatic cells. Organisms of any species, including, but not limited to: yeast, worms, flies, fish, reptiles, birds, mammals (e.g., mice, rats, rabbits, guinea pigs, pigs, micro-pigs, and goats), and non-human primates (e.g., baboons, monkeys, chimpanzees) may be used in the methods described herein. Transgenic mouse models of AD can be used in the methods described herein. For example, transgenic mice expressing a human or mouse APP or presenilin can be used. Some of these transgenic mice develop a progressive neurologic disorder generally within a year from birth (see, e.g., U.S. Pat. No. 5,877,399; U.S. Pat. No. 6,037,521; U.S. Pat. No. 5,894,078; U.S. Pat. No. 5,850,003; and U.S. Pat. No. 5,898,094).

A transgenic cell or animal used in the methods described herein can include a transgene that encodes, e.g., a copy of a SIRT1, e.g., the SIRT1 polypeptide that was evaluated for an interaction with the test compound. The transgene can encode a protein that is normally exogenous to the transgenic cell or animal, including a human protein, e.g., a human SIRT1 polypeptide. The transgene can be linked to a heterologous or a native promoter. Methods of making transgenic cells and animals are known in the art.

Accordingly, in another embodiment, the invention features a method of identifying a compound as a candidate of treatment of neuronal damage, e.g., amyloid-induced neuronal apoptosis, e.g., AD. The method includes: providing a compound which interacts with, e.g., binds to or inhibits deacetylation activity of, SIRT1; and evaluating the effect of the compound on apoptosis, wherein a compound that decreases apoptosis is subjected to further evaluation steps; and further evaluating the effect of the test compound on a subject, e.g., an animal model, e.g., an animal model for AD or a human with AD.

The interaction between a test compound and the SIRT1 polypeptide can be performed by any of the methods described herein, e.g., using cell-based assays or cell-free in vitro assays.

Assays Related to Alzheimer's Disease

Many assays (in addition to the assays described above) can be used to analyze a compound for an effect on AD. For example, cell-based assays can be used to analyze beta-secretase activity and/or processing of APP to release A-beta. Contact of an APP substrate with a beta-secretase enzyme within the cell and in the presence or absence of a compound inhibitor described herein can be used to demonstrate beta-secretase inhibitory activity of the compound. A useful inhibitory compound can provide at least 30% inhibition of the enzymatic activity, as compared with a non-inhibited control.

Cells that naturally express beta-secretase can be used to measure APP processing. Alternatively, cells that are modified to express a recombinant beta-secretase or synthetic variant enzyme are used. The APP substrate may be added to the culture medium and is preferably expressed in the cells. Cells that naturally express APP, variant or mutant forms of APP (e.g., the Swedish mutation), or cells transformed to express an isoform of APP, mutant or variant APP, recombinant or synthetic APP, APP fragment, or synthetic APP peptide or fusion protein containing the beta-secretase APP cleavage site can be used. In a typical assay, the expressed APP is permitted to contact the enzyme and enzymatic cleavage activity can be analyzed.

Human cell lines that normally process A beta from APP provide a useful means to assay inhibitory activities of the compounds described herein. Production and release of A beta and/or other cleavage products into the culture medium can be measured, for example by immunoassay, such as Western blot or enzyme-linked immunoassay (EIA) such as by ELISA.

Cells expressing an APP substrate and an active beta-secretase can be incubated in the presence of a test compound to evaluate modulation (e.g., inhibition) of secretase enzymatic activity, e.g., as compared with a control. Activity of beta-secretase can be measured by analysis of one or more cleavage products of the APP substrate. For example, inhibition of beta-secretase activity against the substrate APP would be expected to decrease release of specific beta-secretase induced APP cleavage products such as A beta. A test compound might effect activity directly or indirectly. For example, a test compound might modulate beta-secretase or APP expression, translation, or degradation.

Although both neural and non-neural cells process and release A-beta, levels of endogenous beta-secretase activity may be low and can be difficult to detect by immunoassays in at least some systems. The use of cell types known to have enhanced beta-secretase activity, enhanced processing of APP to A beta, and/or enhanced production of A beta are therefore preferred. For example, transfection of cells with the Swedish Mutant form of APP (APP-SW); with APP-KK; or with APP-SW-KK provides cells having enhanced beta-secretase activity and producing amounts of A beta that can be readily measured.

In such assays, for example, the cells expressing APP and beta-secretase are incubated in a culture medium under conditions suitable for beta-secretase enzymatic activity at its cleavage site on the APP substrate. On exposure of the cells to an inhibitor, the amount of Abeta released into the medium and/or the amount of CTF99 fragments of APP in the cell lysates is reduced as compared with the control. The cleavage products of APP can be analyzed, for example, by immune reactions with specific antibodies, as discussed above.

Cells useful for analysis of beta-secretase activity include primary human neuronal cells, primary transgenic animal neuronal cells where the transgene is APP, and other cells such as those of a stable 293 cell line expressing APP, for example, APP-SW.

In Vivo AD Assays: Animal Models

Various animal models can be used to analyze beta-secretase activity and/or processing of APP to release A beta, as described above. For example, transgenic animals expressing APP substrate and beta-secretase enzyme can be used to demonstrate inhibitory activity of a compound. Certain transgenic animal models have been described, for example, in U.S. Pat. Nos. 5,877,399; 5,612,486; 5,387,742; 5,720,936; 5,850,003; 5,877,015, and 5,811,633, and in Ganes et. al., 1995, Nature 373:523. Preferred are animals that exhibit characteristics associated with the pathophysiology of AD. Administration of inhibitors to the transgenic mice described herein provides an alternative method for demonstrating the inhibitory activity of the compounds. Administration of the compounds in a pharmaceutically effective carrier and via an administrative route that reaches the target tissue in an appropriate therapeutic amount is also preferred.

Inhibition of beta-secretase mediated cleavage of APP at the beta-secretase cleavage site and of A beta release can be analyzed in these animals by measure of cleavage fragments in the animal's body fluids such as cerebral fluid or tissues. Analysis of brain tissues for A beta deposits or plaques is preferred.

On contacting an APP substrate with a beta-secretase enzyme in the presence of an inhibitory compound and under conditions sufficient to permit enzymatic mediated cleavage of APP and/or release of A beta from the substrate, useful compounds can be effective to reduce beta-secretase-mediated cleavage of APP at the beta-secretase cleavage site and/or effective to reduce released amounts of A beta. Where such contacting is the administration of the inhibitory compounds to an animal model, for example, as described above, the compounds are effective to reduce A beta deposition in brain tissues of the animal, and to reduce the number and/or size of beta amyloid plaques. Where such administration is to a human subject, the compounds are effective to inhibit or slow the progression of disease characterized by enhanced amounts of A beta, to slow the progression of AD in the, and/or to prevent onset or development of AD in a patient at risk for the disease.

Pharmaceutical Compositions

The invention includes methods of modulating SIRT1 activity, e.g., to treat or prevent Alzheimer's Disease. The method can include administering to a cell or an organism a compound that interacts with SIRT1 and effects SIRT1 activity. For example, the compound can be a SIRT1 agonist. The compound may modulate (e.g., inhibit) apoptosis, e.g., in a neuronal cell.

The compound can be administered to human or a human cell, e.g., a human neuron. The compound can also be administered to other types of cells and organisms, e.g., for evaluation in in vitro or in animal models of AD. For example, the cell to which the compound is administered can be an invertebrate cell, e.g., a worm cell or a fly cell, or a vertebrate cell, e.g., a fish cell (e.g., zebrafish cell), or a mammalian cell (e.g., mouse). Similarly, the organism to which the compound is administered can be an invertebrate, e.g., a worm or a fly, or a vertebrate, e.g., a fish (e.g., zebrafish), an amphibian, or a mammal (e.g., rodent).

The compound that is administered to the cell or organism is an agonist that increases the expression or activity of the SIRT1 polypeptide, thereby decreasing apoptosis of the cell. The compound can be a small organic compound, an antibody, a polypeptide, or a nucleic acid molecule. An exemplary SIRT1 agonist is a nucleic acid that encodes a protein with a SIRT1 activity, e.g., a fragment of SIRT1 with an active catalytic site or a full-length SIRT1, or a complement thereof. Another agonist is a SIRT1 cofactor (e.g., NAD), or another small organic compound, e.g., which activates SIRT1 or stimulates its activity. Other agonists can prevent SIRT1 degradation or may modulate SIRT1 intracellular localization.

One exemplary class of SIRT1 agonists include polyphenols, e.g., a flavone, stilbene, flavanone, cetchin, chalcone, isoflavone, anthocyanidin, or tannin.

In some instances, the SIRT1 agonist is a compound of formula (I) 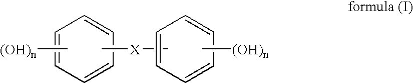
wherein;
X is alkenyl, C(O)CH═CH, or a hydroxy pyranone fused to one of the phenyl moieties to form a flavone; and
each n is independently 1-3.

For example, the compound can be a polyhydroxy stillbene (e.g., polyhydroxy-trans-stillbene) as shown in formula (II), a polyhydroxy chalcone as shown in formula (III), or a polyhydroxyflavone as shown in formula (IV). In general, the compound is substituted with at least 2, preferably 3, 4, of 5 hydroxy moieties.

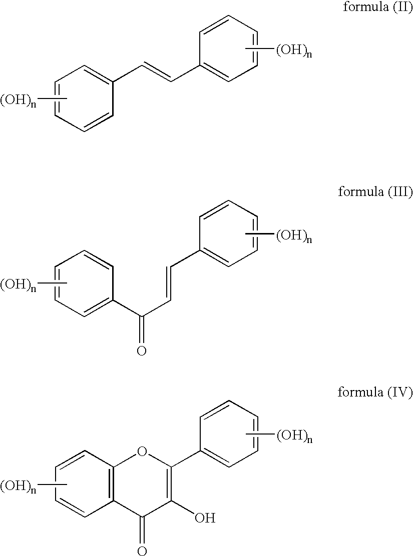

Exemplary compounds include resveratrol (3,5,4′-trihydroxy-tans-stilbene), butein (3,4,2′,4′- tetrahydroxychalcone); piceatannol (3,5,3′,4′-tetrahydroxy-trans-stilbene); isoliquiritigenin (4,2′,4′-trihydroxychalcone); fisetin (3,7,3′,4′-tetrahydroxyflavone); and quercetin (3,5,7,3′,4′-pentahydroxyflavone). See, e.g., Howitz (2003) Nature 425:191-196. In one embodiment, such compounds are provided in a non-liquid form, e.g., a semi-solid form, e.g., a tablet or gel. In another embodiment, the compounds is in liquid form, e.g., a beverage, e.g., a non-alcoholic beverage, e.g., a beverage that does or does not include a natural by product of grapes.

Antibodies that are both specific for a target gene protein and that interfere with its activity may be used to inhibit finction of a target protein, e.g., a negative regulator of SIRT1 (e.g., the SIRT1 gene or protein). Antibodies can also be used as SIRT1 agonists, e.g., an antibody may inhibit an inhibitor of SIRT1 or may bind to SIRT1 and increase SIRT1 activity, e.g., by stabilizing an active conformation of SIRT1. Such antibodies may be generated using standard techniques, against the proteins themselves or against peptides corresponding to portions of the proteins. Such antibodies include but are not limited to polyclonal, monoclonal, Fab fragments, single chain antibodies, chimeric antibodies, humanized antibodies and the like. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (e.g., see Sambrook et al., Eds., Molecular Cloning: A Laboratory Manual, 2nd ed., Cold Spring Harbor Laboratory Press, (1989), or Ausubel, F. M. et al., eds. Current Protocols in Molecular Biology (1994).

The SIRT1 agonist can also be a siRNA, anti-sense RNA, or a ribozyme which can increase the expression of the SIRT1 polypeptide (e.g., by inhibiting expression of a negative regulator of SIRT1 protein). Double-stranded inhibitory RNA is particularly useful as it can be used to selectively reduce the expression of one allele of a gene and not the other, thereby achieving an approximate 50% reduction in the expression of a SIRT1 antagonist polypeptide. See Garrus et al. (2001), Cell 107(1):55-65. Thus, in some aspects, a cell or subject can be treated with a compound that modulates the expression of a gene, e.g., a nucleic acid which modulates, e.g., decreases, expression of a polypeptide which inhibits SIRT1. Such approaches include oligonucleotide-based therapies such as RNA interference, antisense, ribozymes, and triple helices.

dsRNA can be delivered to cells or to an organism to agonize SIRT1. Endogenous components of the cell or organism trigger RNA interference (RNAi) which silences expression of genes that include the target sequence. dsRNA can be produced by transcribing a cassette (in vitro or in vivo) in both directions, for example, by including a T7 promoter on either side of the cassette. The insert in the cassette is selected so that it includes a sequence complementary to a nucleic acid encoding a negative regulator of SIRT1. The sequence need not be full length, for example, an exon, or at least 50 nucleotides. The sequence can be from the 5′ half of the transcript, e.g., within 1000, 600, 400, or 300 nucleotides of the ATG. See also, the HISCRIBE™ RNAi Transcription Kit (New England Biolabs, MA) and Fire, A. (1999) Trends Genet. 15, 358-363. dsRNA can be digested into smaller fragments. See, e.g., US Patent Application 2002-0086356 and 2003-0084471. In one embodiment, an siRNA is used. siRNAs are small double stranded RNAs (dsRNAs) that optionally include overhangs. For example, the duplex region is about 18 to 25 nucleotides in length, e.g., about 19, 20, 21, 22, 23, or 24 nucleotides in length. Typically the siRNA sequences are exactly complementary to the target mRNA. The siRNA sequence can be design to target only SIRT1 and not other sirtuins. The sequences of the different human sirtuins are known. The siRNA sequence can target a conserved region of a SIRT1 nucleic acid, e.g., a region conserved between human and mouse, or between human and another model organism. In one embodiment, the siRNA sequence targets a region of a SIRT1 nucleic acid that encodes a part of the sirtuin homology domain, e.g., about amino acids 214-541.

Oligonucleotides may be designed to reduce or inhibit mutant target gene expression and/or activity. Techniques for the production and use of such molecules are well known to those of ordinary skill in the art. Antisense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the target gene nucleotide sequence of interest, are preferred. Antisense oligonucleotides are preferably 10 to 50 nucleotides in length, and more preferably 15 to 30 nucleotides in length. An antisense compound is an antisense molecule corresponding to the entire mRNA of the target gene or fragments thereof.

Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. The composition of ribozyme molecules includes one or more sequences complementary to the target gene mRNA, and includes the well known catalytic sequence responsible for mRNA cleavage disclosed, for example, in U.S. Pat. No. 5,093,246. Within the scope of this disclosure are engineered hannnerhead motif ribozyme molecules that specifically and efficiently catalyze endonucleolytic cleavage of RNA sequences encoding target gene proteins. Specific ribozyme cleavage sites within any potential RNA target are initially identified by scanning the molecule of interest for ribozyme cleavage sites that include the sequences GUA, GUU, and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

The antisense, ribozyme, and/or triple helix molecules described herein may reduce or inhibit the transcription (triple helix) and/or translation (antisense, ribozyme) of mRNA produced by both normal and mutant target gene alleles.

Antisense RNA and DNA, ribozyme, and triple helix molecules may be prepared by any method known in the art for the synthesis of DNA and RNA molecules. These include techniques for chemically synthesizing oligodeoxyribonucleotides and oligoribonucleotides, for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Various well-known modifications to the DNA molecules may be introduced as a means of increasing intracellular stability and half-life. Possible modifications include but are not limited to the addition of flanking sequences of ribonucleotides or deoxyribonucleotides of the 5′ and/or 3′ ends of the molecule or the use of phosphorothioate or 2′ O-methyl rather than phosphodiesterase linkages within the oligodeoxyribonucleotide backbone.

Delivery of nucleic acids can be achieved using a recombinant expression vector such as a chimeric virus or a colloidal dispersion system or by injection. Useful virus vectors include adenovirus, herpes virus, vaccinia, and/or RNA virus such as a retrovirus. The retrovirus can be a derivative of a murine or avian retrovirus such as Moloney murine leukemia virus or Rous sarcoma virus. All of these vectors can transfer or incorporate a gene for a selectable marker so that transduced cells can be identified and generated. The specific nucleotide sequences that can be inserted into the retroviral genome to allow target specific delivery of the retroviral vector containing an antisense oligonucleotide can be determined by one of skill in the art.

Another delivery system for polynucleotides is a colloidal dispersion system. Colloidal dispersion systems include macromolecular complexes, nanocapsules, microspheres, beads, and lipid-based systems including oil-in-water emulsions, micelles, mixed micelles and liposomes. A preferred colloidal delivery system is a liposome, an artificial membrane vesicle useful as in vivo or in vitro delivery vehicles. The composition of a liposome is usually a combination of phospholipids, usually in combination with steroids, particularly cholesterol.

The identified compounds that modulate (e.g., activate) SIRT1 activity, e.g., SIRT1 gene expression, synthesis and/or activity (or inhibit expression of a target gene product that inhibits SIRTL) can be administered to a patient at therapeutically effective doses to treat or ameliorate or delay one or more of the symptoms of AD. A therapeutically effective dose refers to that amount of the compound sufficient to result in amelioration or delay of one or more of the symptoms of AD.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50 /ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in a method described herein, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Pharmaceutical compositions may be formulated in conventional manner using one or more physiologically acceptable carriers or excipients. Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compounds identified by the methods described herein can be used in the treatment of diseases or conditions associated with Alzheimer's Disease. The compounds can be administered alone or as mixtures with conventional excipients, such as pharmaceutically, or physiologically, acceptable organic, or inorganic carrier substances such as water, salt solutions (e.g., Ringer's solution), alcohols, oils and gelatins. Such preparations can be sterilized and, if desired, mixed with lubricants, preservatives, stabilizers, wetting agents, emulsifiers, salts for influencing osmotic pressure, buffers, coloring, and/or aromatic substances and the like.

Therapeutic Uses

The invention includes methods for treating or preventing a disease in which SIRT1 is implicated, e.g., AD, in a subject. The method includes administering a SIRT1 agonist. For example, the SIRT1 agonist can be one or more of: a SIRT1 nucleic acid, RNAi (e.g., RNAi targeted to a molecule that inhibits SIRT1), and other compounds identified by a method described herein, e.g., compounds that reduce apoptosis in a cell.

“Subject,” as used herein, refers to human and non-human animals. The term “non-human animals” includes all vertebrates, e.g., mammals, such as non-human primates (particularly higher primates), sheep, dog, rodent (e.g., mouse or rat), guinea pig, goat, pig, cat, rabbits, cow, and non-mammals, such as chickens, amphibians, reptiles, etc. In a preferred embodiment, the subject is a human, e.g., an AD patient. In another embodiment, the subject is an experimental animal or animal suitable as a disease model.

In a preferred embodiment, the method includes administering a SIRT1 agonist in combination with one or more additional therapeutic agent or agents, e.g., a therapeutic agent or agents for treating AD.

The SIRT1 agonists described herein, e.g., compounds which interact with SIRT1 and effect apoptosis (e.g., compounds identified by the methods described herein), can be used in combination with other therapies. For example, the combination therapy can include a SIRT1 agonist of the present invention co formulated with, and/or co administered with, one or more additional therapeutic agents, e.g., one or more AD therapeutic agents. Such combination therapies may advantageously utilize lower dosages of the administered therapeutic agents, thus avoiding possible toxicities or complications associated with the various monotherapies. In some embodiments, delivery is such that the reduction in a symptom, or other parameter related to the disorder, is greater than what would be observed with the second treatment delivered in the absence of the SIRT1 antagonist. The effect of the two treatments can be partially additive, wholly additive, or greater than additive. In some embodiments, the administration of an anti-AD agent in combination with a SIRT1 agonist, may lower the dose of the anti-AD agent or other therapeutic agents by at least 2, 3, 4, 5, 10, 15, 20, 30, 40, 50, 60% or more from the dose of the anti-AD agent or other therapeutic agent administered in the absence of administration of the SIRT1 antagonist.

Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. The delivery can be such that an effect of the first treatment delivered is still detectable when the second is delivered.

### EXAMPLE

Long-lived individuals (e.g. centenarians and nonagenarians), can provide a reference genome for identifying genes involved in aging and the diseases of aging, e.g., Alzheimer's Disease. Average life expectancy for long-lived individuals (LLI) born near the turn of the 19th century, prior to advances such as antibiotics, chemotherapy, and cholesterol-lowering medication was only about 50 years. Surviving the entire 20th century likely required a rare combination of luck, environment, and genetic constitution. With respect to the last, twin studies suggest that genetics accounts for about 25% of the variance in human lifespan (Herskind, A. M. et al. Hum Genet 97:319-23, 1996). The genetic impact may be more significant at the extremes of age; male and female siblings of centenarians have a 17 times and 8 times greater probability, respectively, of living to 100 years compared to average members of their birth cohorts (Perls, T. et al. Proc Natl Acad Sci USA 99:8442-8447,2002). Conceptually, healthy LLI either markedly delay or escape age-associated conditions such as Alzheimer's Disease (AD), cardiovascular disease, stroke, diabetes, and cancer. This may involve inheriting a protective mix of genetic variants that mitigates susceptibility to age related diseases. For example, multiple groups have demonstrated that LLI have a low frequency of an AD predisposing apolipoprotein E (apo-E) ε-4 allele and a high frequency of an AD protective ε-2 allele (Finch, C. E. & Tanzi, R. E. Science 278:407-11,1997; Skoog, I. et al. J Neurol Neurosurg Psychiatry 64:37-43,1998; Smith, J. D. Ageing Res Rev 1:345-65,2002).

The unique profile of LLI makes them an ideal genetic foil to individuals affected by the diseases of aging and can be used to identify important genes that modulate susceptibility to human age-related diseases such as AD. Typically, in genetic association studies, genes of interest are tested in large groups of AD patients compared with a group of age-matched controls. A statistical difference in allele frequency between the two groups supports the notion that the gene and the disease are somehow related. However, many of the individuals in the control group may have “silent” copies of the disease-predisposing gene and may eventually develop AD. This contamination of the control group decreases statistical power. Individuals surviving to extremes of age who remain cognitively intact should be less likely to possess risk alleles, and therefore the genetic contrast between LLI and AD samples should be greater than between age-matched controls and AD samples. This increased contrast provides a greater likelihood of detecting an association, for a given sample size.

Allele frequencies of approximately 50 genes of 760 LLI can be compared to 760 AD patients within the 17 million base-pair genetic locus on chromosome 10q previously implicated by multiple linkage studies in late-onset AD (Ertekin-Taner, N. et al. Science 290:2303-4,2000; Myers, A. et al. Science 290:2304-5, 2000). Although these linkage studies provided genome-wide screens for AD, the resolution of this mapping technique is on the order of tens of millions of base pairs. Genetic association studies are an accepted way to fine map a linkage locus and identify implicated gene variants.

Identifying the genetic variants modulating susceptibility to AD will add to our understanding of this pathology and advance progress towards effective treatments. As LLI are models for delayed onset of aging-related diseases, contrasting this group with AD-affecteds can identify a risk gene.

Relative to the general population, LLI can have a genetic predisposition to live longer than the general population and either markedly delay or escape age-associated diseases such as AD, cardiovascular disease, stroke, diabetes, and cancer. The great majority of the LLI cohort in this study was cognitively intact well into their late 80s. There is accumulating evidence that centenarians pass on this protective trait to their offspring (8). In addition to lacking genetic variants that predispose to disease (risk alleles), they may also possess genetic variations that slow the aging process and decrease susceptibility to the diseases of aging (protective alleles)(9). Specifically, cognitively intact LLI should be underrepresented in AD predisposing alleles and/or overrepresented in AD protective alleles. Furthermore, this difference in allele frequency may be greater than that observed between AD patients and age-matched controls (i.e. cognitively intact individuals the same average age as the AD individuals). A number of these genetic variations can be discovered through the systemic mapping of genetic linkage peaks using LLI association studies. This represents a unique approach to uncovering novel and basic biologic mechanisms that modulate susceptibility to many age-related diseases, including AD.

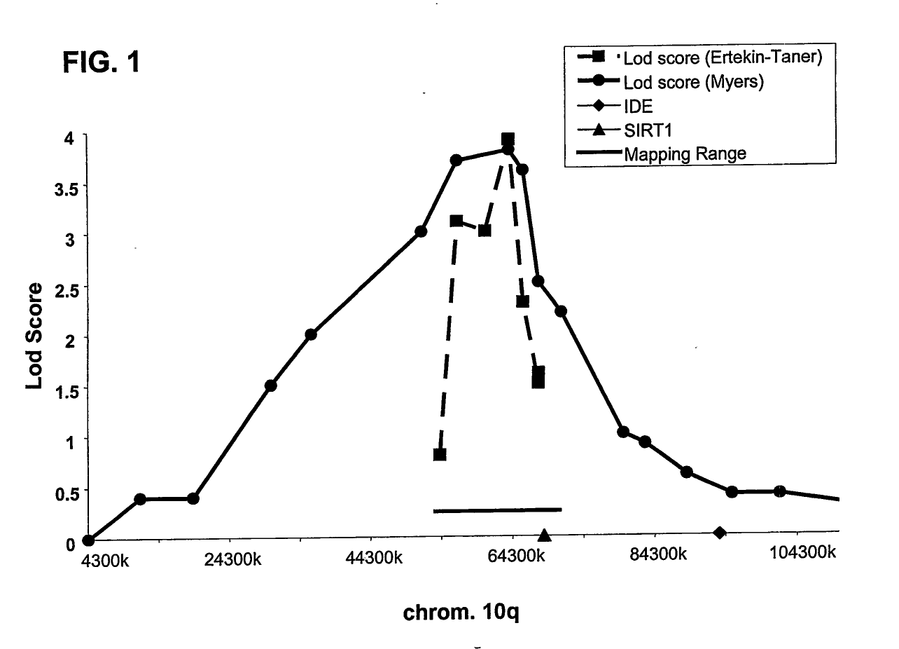

Multiple independent studies have implicated a consistent region of chromosome 1 Oq in late onset AD (6),(7). FIG. 1 superimposes the two linkage peaks from these studies. To date, the gene underlying this peak has not been unambiguously identified. Others have reported that Insulin Degrading Enzyme (IDE) may account for the observed linkage (10), and there is functional biology implicating IDE with AD (11). However, IDE lies in the tail of the linkage peak (see FIG. 1), the statistical evidence for association is weak, and not all groups have been able to reproduce the result (1 1),(12).

In the approximately 17 million base pairs spanning the overlapping portions of the two linkage peaks (see designated locus in FIG. 1), there are approximately 50 well-characterized genes as of the November 2002 human genome draft.

This study uses a large collection of DNA and phenotypic data from LLI (approximately 44% of these 1600 samples were obtained from centenarians) and a novel algorithm for matching the genetic backgrounds of the samples compared in genetic association studies. This reduces the high false positive rate from genetic stratification of the contrasted sample sets (13). Genetic single nucleotide polymorphism (SNP) markers in large samples of LLI and AD-affected patients can be compared. As has been demonstrated in the case of apolipoprotein E, a gene can be identified with alleles that vary in frequency between the LLI and AD patients.

The gene SIRT1 lies directly under the AD linkage peak (see FIG. 1). The yeast and worm orthologs of SIRT1 have been shown to have a role in modulating lifespan (14). The mouse ortholog of SIRT1 interacts with p53 (15) and may have a role in the pathogenesis of AD through a modulating effect on apoptosis and oxidative stress (15),(16). The identification of SIRT1 as a possible contributor to the incidence and progression of AD allows the use of SIRT1-modulating compounds to further investigate the mechanisms of AD and to begin testing the efficacy of these compounds in cellular and animal models for AD.

1. Establish collection of AD patients that is genetically well matched to a cognitively intact subgroup of LLI.

DNA is obtained from 760 late-onset AD patients to compare with the LLI; one source for these AD samples is the National Institute of Mental Health (NIMH). A group of 1140 LLI are selected from a database based on a heuristic to identify unrelated individuals who are the oldest and most cognitively intact. Using an algorithm described below, a sample subset (n=760) is selected to best match the genetic background of the 760 AD samples.

2. Use LLI and AD samples to identify the gene responsible for the chromosome 10 q AD linkage peak.

LLI and AD samples can be compared to look for statistically significant allele frequency differences in a genetic association study. Previous experience has suggested that 5-10 validated SNPs per gene are sufficient as an initial screen for a putative association.

With approximately 300 validated SNP markers, genotyping of 380 well-matched and unrelated LLI and 380 AD affected samples randomly selected from the larger well-matched pool can be genotyped. Using a multivariate test of association (Hotelling T test, see Methods), alleles at each of the 50 genes are statistically compared. The genes with the lowest “p” values that emerge from this first screening tier, can be retested in the remaining set of 380 LLI and 380 AD-affected samples. The individual SNP frequencies are examined for any retested gene showing association at p<0.10 by Hotelling. If the SNP frequency differences are consistent between the two tiers, the implicated gene can be densely genotyped using additional SNPs from public databases, with a goal of one SNP for every 1-2 kb. Validated SNPs can be run on all samples from both tiers and a haplotype map of the region can be constructed using the Expectation Maximization (EM) algorithm (17),(18). From this map, the most likely risk haplotype allele can be identified based on p-value and relative risk.

3. Identify potentially causative SNP polymorphisms within the implicated gene.

Publicly available SNP markers can be used to identify the haplotype that varies most in frequency between the LLI and AD samples. Single SNPs which uniquely “tag” or distinguish this haplotype from other haplotypes within the block are potentially “causative” polymorphisms under the most parsimonious model that a single SNP accounts for the trait variance between two sample sets. To identify all potentially causative SNPs, multiple samples can be re-sequenced and novel SNPs can be identified in the vicinity of the risk haplotype. Special attention can be paid to identifying novel polymorphisms in genomic areas more likely to have functional significance (exons, promoter, regulatory sequences, etc.) This group of SNPs can then be validated and subsequently assayed in all 760 cases and controls. These additional data can be used to supplement a haplotype map.

It is important to note that this study may not be able to implicate a single SNP as the sole causative variant, nor is it necessary to do so. There are many nearby SNPs which are statistically equivalent to one another (correlation coefficient of 1.0) because of linkage disequilibrium and although statically indistinguishable, one may be more interesting than the others because of non-statistical arguments (e.g. a base pair change resulting in a non-conservative amino acid substitution is more likely to have functional implications than a SNP within an intron.) In addition, a single SNP may not explain the trait variance at a locus. Multiple polymorphisms and interactions between these polymorphisms may contribute to the statistical distortion underlying this linkage peak.

Evidence supportive of a genetic component to exceptional longevity and genetic determinants of decreased susceptibility to or protection from age-related diseases such as AD

If extreme lifespan has a partial genetic basis, part of this protection from age-related pathology may also be transmitted to the siblings of LLI, who should have reduced mortality rates compared with other individuals in their birth cohorts. To test this hypothesis, 444 centenarian pedigrees containing 2,092 siblings(2) were studied. Sibling death rates and survival probabilities were compared to U.S. national levels. Comparison of the death rates of the siblings of centenarians relative to the control cohort reveals a life-long sustained reduction of mortality risk by approximately one half. In addition, a relatively constant advantage from moment to moment is translated into an increasing survival advantage over a lifetime (relative survival probabilities—RSP); ultimately the brothers of centenarians had a 17 times greater probability of achieving 100 and the sisters had an 8 times greater probability. The substantially higher RSP values for men at older ages is consistent with the hypothesis that, relative to women, men require a greater contribution of this genetic advantage to achieve extreme ages. The sustained advantage observed by siblings of centenarians is unusual, suggesting a persistent etiology such as genetic makeup.

Affected Only Sibling Pair Linkage Study: A genome-wide scan for longevity predisposing loci studying 308 centenarians belonging to 137 sibships (19) was performed. Using non-parametric analysis, significant evidence for linkage was noted for chromosome 4 at D4S1564 with a MLS of 3.65 (p=0.044). The analysis was corroborated by a parametric analyses (p=0.052). These linkage results suggest that there exists a gene or genes that exert a substantial influence upon the ability to achieve exceptional old age while necessarily escaping or delaying high mortality diseases.

Follow-up Association Study: The linkage study was followed by a large genotyping effort, systemically mapping 12 million base pairs under the linkage peak. After constructing a haplotype map for this 12 million base pair region, LLI were compared to controls at each of these markers. This approach yielded an allele with statistical association to extreme longevity that can explain the earlier linkage result (28).

TABLE 1

| | LLI Allelic | Controls Allelic |
|---|---|---|
| Apo E allele | Frequency | Frequency |
| Apo ε-2 | 11% | 7% |
| Apo ε-3 | 82% | 81% |
| Apo ε-4 | 7% | 12% |

Successful candidate gene longevity associations: Apolipoprotein E has been implicated in the pathogenesis of AD in multiple association studies(4),(24),(25). In addition, the decreased frequency of the ε-4 allele and increased frequency of the ε-2 allele among LLI has been previously described(4),(20),(21),(23-25). In a study of 800 LLI and 800 controls, this highly significant association (p<0.000001) was replicated with respect to three apolipoprotein E alleles (Table 1)(28).

These preliminary data show that LLI is a useful model for delayed or escaped AD and validate the use of a well-matched collection for the identification and implication of gene variants in the incidence and progress of disease. These studies indicate that careful association studies between LLI and controls can yield genetic variations associated with exceptionally longevity, and similar comparisons between LLI and patients who have AD provide information about polymorphisms (in the SIRT1 gene and elsewhere) that are protective against AD as well as polymorphisms that predispose to the disease. AD-related polymorphisms in novel genes can then be used as new screening targets for drug discovery, and polymorphisms in known genes can be used to design more powerful screens or, if the structure of the protein is known, be used in structure-aided drug design projects.

Experimental Design and Methods

LLI ascertainment: LLI can be identified by a variety of methods. Neither physical nor cognitive health is used as participation criteria. Age is the only inclusion criteria (Males≧90 yrs; Females≧94 yrs). All participants and/or their legally authorized representatives take part in the informed consent process, as required by the Institutional Review Boards overseeing the aforementioned organizations.

AD-affected ascertainment: A number of potential sources of DNA from AD patients have been identified. These sources include governmental and commercial AD biorepositories as well as collections from well-established academic institutions. Prior to utilizing samples from these sources, samples are collected according to state and federal guidelines with regards to human subjects' protection and privacy and that the AD diagnosis has been made utilizing well-established accepted criteria. Namely, a diagnosis made after age 65 following the National Institute of Health (NIH)/Alzheimer's Association Work Group (NINCDS-ADRDA) guidelines. This involves physical and neurological examination (e.g., MRI/CT scan) accompanied by cognitive testing (e.g., MMSE, ADAS) to identify the characteristic memory, language and other cognitive and non-cognitive symptoms of AD.

Control of confounders/stratification:

Stratification between the LLI with AD-affecteds samples can cause the identification of allele frequency differences that reflect variance in ethnicity rather than resistance to AD. Although there are published methods of checking and correcting for this potential confounder, these methods result in a loss of power and make statistical distribution assumptions which have not been validated(26),(27).

To address this problem, we have developed a novel algorithm to select subgroups of samples that are genetically well matched with respect to a reference sample set. In the current study, we will genotype 60 independent SNP markers in the 1140 cognitively intact LLI and 760 AD-affected samples. This gives an LLI/AD pool size ratio of 3:2. The genotypes will be numerically coded as follows: −1 for minor SNP allele homozygotes, 0 for heterozygotes, 1 for major SNP allele homozygotes. A subgroup of 760 LLI will be selected that minimizes the Mahalanobis distance (D) with respect to the AD samples:
D²=( V̅₁− V̅₂)S⁻¹( V̅₁− V̅₂)^{r}
where V̅₁ is a 60 dimensional vector representing the mean genotyping values of the LLI, V̅₂ is the equivalent vector for the AD-affected, and S⁻¹ is the inverse of the corresponding 60 by 60 covariance matrix. This proactive sample matching method has been used to establish well-matched sets of cases and controls in samples where initially significant stratification was detected. We will confirm that there is no remaining residual stratification by genotyping the 1520 samples at 60 new SNP markers and testing for stratification with an established method, described below(26).

Testing for stratification: Genotyping an additional set of 60 random SNP markers in the proactively matched AD-affected and LLI samples can confirm the absence of stratification. For each marker, one can construct a 2×2 contingency table comparing allele counts between the two groups and calculate 60 chi-square statistics for each test of association. Because these SNPs were selected at random across the genome and are uncorrelated, systematic differences in allele frequencies would infer differences in genetic backgrounds between AD-affected and LLI samples. If the genetic backgrounds of the two armed study were perfectly matched, the mean chi-square for these 60 comparisons will have an expected value of 1.0. If the chi-square mean departs significantly from unity, the proactive sample matching process will be repeated using a larger LLI/AD pool size ratio.

SNP assay and validation methods: Potential SNPs can be retrieved from the Human Genome Draft Database. Assays were designed using spectroDESIGNER software (Sequenom) to be multiplexed up to five times. SNP genotyping can be performed on Sequenom's chip-based matrix-assisted laser desorption/ionization time-of-flight (MALDI-TOF) mass spectrometry (DNA MassARRAY) on PCR-based extension products from individual DNA samples. AD and LLI samples can be run on the same chip to avoid potential artifacts due to chip-specific miss-calls.

Statistical tests of association: The G-Test with Williams correction (a statistic following a chi-square distribution) can be used to test inferences about associating genetic markers (haplotype or SNP based) with AD or the longevity phenotype. For each allele, 2×2 contingency tables are constructed as +/− allele vs. +/− AD. During the screening tier of the proposed investigation, we require a summary statistic (1 gene, one test) to reflect the probability each gene's alleles are asymmetrically distributed between the two sample sets being contrasted. To maximize statistical power while testing 5-10 SNP markers per gene, we exploit a permutation test using the Hotelling statistic to compare the multivariate genotypic population means at each gene. The permutation version of this test is resistant to the inevitable missing genotype data in these studies.

SNP selection and validation: For each of the 50 genes under the peak, 10 SNPs can be selected from publicly available databases such as the SNP Consortium (TSC) website (http://snp.cshl.org). As approximately 40% of these markers might either not be polymorphic or not amenable to genotyping on our platform, one can first validate the markers on 25 control samples. SNPs with minor allele frequencies less than 5%, SNPs not in Hardy/Weinberg equilibrium, and SNPs with less than 80% call rate will be excluded from further consideration.

Sequencing: Standard Applied Biosystems sequencing kits can be used for sequencing. This will be followed by analysis on an ABI 3700 96 capillary sequencer. All samples can be prepared in a 96 well format using robotic workstations to perform pipeting. Phred program (by Codoncode) can be used for quality scores and Sequencer (by Genecodes) for sequence comparisons and SNP detection.

Haplotype reconstruction: For each sample and each SNP assay, one can determine phase where possible (i.e. in homozygotes). Missing data or phase ambiguous data can be resolved using the Expectation Maximization (EM) algorithm(l 7),(18). A haplotype is defined as a contiguous region of DNA with little evidence (<2.5%) for meiotic recombination within the common genetic history of the individuals genotyped. In situations where the boundaries are ambiguous, a second heuristic can be applied that assigned boundaries in such as way to minimize the size (i.e. base pairs) within each block. With haplotype boundaries assigned, haplotype frequencies are estimated for each haplotype allele using the EM algorithm. Any haplotype that has a frequency of less than 2.5% will be excluded from further analysis to avoid possible errors in either the genotyping or the estimation process.

### F. REFERENCES

1. Herskind, A. M. et al. The heritability of human longevity: a population-based study of 2872 Danish twin pairs born 1870-1900. Hum Genet 97, 319-23 (1996).
2. Perls, T. et al. Life-long sustained mortality advantage of siblings of centenarians. Proc Natl Acad Sci USA 99, 8442-8447 (2002).
3. Finch, C. E. & Tanzi, R. E. Genetics of aging. Science 278, 407-11 (1997).
4. Skoog, I. et al. A population study of apoE genotype at the age of 85: relation to dementia, cerebrovascular disease, and mortality. J. Neurol Neurosurg Psychiatry 64, 37-43 (1998).
5. Smith, J. D. Apolipoproteins and aging: emerging mechanisms. Ageing Res Rev 1, 345-65 (2002).
6. Ertekin-Taner, N. et al. Linkage of plasma Abeta42 to a quantitative locus on chromosome 10 in late-onset Alzheimer's disease pedigrees. Science 290, 2303-4 (2000).
7. Myers, A. et al. Susceptibility locus for Alzheimer's disease on chromosome 10. Science 290, 2304-5 (2000).
8. Terry, D., Wilcox, M., McCormick, M., Lawler, E. & Perls, T. Cardiovascular Advantages Among the Offspring of Centenarians. Journal Gerontological Medical Science In Press (2003).
9. Yashin, A. I. et al. Genes, demography, and life span: the contribution of demographic data in genetic studies on aging and longevity. Am J Hum Genet 65, 1178-93 (1999).
10. Bertram, L. et al. Evidence for genetic linkage of Alzheimer's disease to chromosome 10q. Science 290, 2302-3 (2000).
11. Farris, W. et al. Insulin-degrading enzyme regulates the levels of insulin, amyloid beta-protein, and the beta -amyloid precursor protein intracellular domain in vivo. Proc Natl Acad Sci USA 100, 4162-7 (2003).
12. Lendon, C. & Craddock, N. Susceptibility gene(s) for Alzheimer's disease on chromosome 10. Trends Neurosci 24, 557-9 (2001).
13. Hirschhorn, J. N., Lohmueller, K., Byrne, E. & Hirschhorn, K. A comprehensive review of genetic association studies. Genet Med 4, 45-61 (2002).
14. Kaeberlein, M., McVey, M. & Guarente, L. The SIR2/3/4 complex and SIR2 alone promote longevity in Saccharomyces cerevisiae by two different mechanisms. Genes Dev 13, 2570-80 (1999).
15. Luo, J. et al. Negative control of p53 by Sir2alpha promotes cell survival under stress. Cell 107, 137-48 (2001).
16. Aguilaniu, H., Gustafsson, L., Rigoulet, M. & Nystrom, T. Asymmetric inheritance of oxidatively damaged proteins during cytokinesis. Science 299, 1751-3 (2003).
17. Excoffier, L. & Slatkin, M. Maximum-likelihood estimation of molecular haplotype frequencies in a diploid population. Mol Biol Evol 12, 921-7 (1995).
18. Long, J. C., Williams, R. C. & Urbanek, M. An E-M algorithm and testing strategy for multiple-locus haplotypes. Am J Hum Genet 56, 799-810 (1995).
19. Puca, A. A. et al. A genome-wide scan for linkage to human exceptional longevity identifies a locus on chromosome 4. Proc Natl. Acad Sci USA 98, 10505-8 (2001).
20. van Bockxmeer, F. M. ApoE and ACE genes: impact on human longevity. Nat Genet 6, 4-5 (1994).
21. Schachter, F. et al. Genetic associations with human longevity at the APOE and ACE loci. Nat Genet 6, 29-32 (1994).
22. Arking, D. E. et al. Association of human aging with a finctional variant of klotho. Proc Natl Acad Sci USA 99, 856-861 (2002).
23. Kervinen, K. et al. Apolipoprotein E and B polymorphisms—longevity factors assessed in nonagenarians. Atherosclerosis 105, 89-95 (1994).
24. Tilvis, R. S., Strandberg, T. E. & Juva, K. Apolipoprotein E phenotypes, dementia and mortality in a prospective population sample. J Am Geriatr Soc 46, 712-5 (1998).
25. Smith, J. D. Apolipoprotein E4: an allele associated with many diseases. Ann Med 32, 118-27 (2000).
26. Reich, D. E. & Goldstein, D. B. Detecting association in a case-control study while correcting for population stratification. Genet Epidemiol 20, 4-16 (2001).
27. Devlin, B., Roeder, K. & Wasserman, L. Genomic control, a new approach to genetic-based association studies. TheorPopulBiol 60, 155-66 (2001).
28. Geesaman B J, Benson E, Brewster S J, Kunkel L M, Perls T T, Daly M J, Puca A A. Haplotype based identification of gene variants modulating human lifespan. Submitted.

A number of embodiments have been described. Nevertheless, it will be understood that various modifications may be made without departing from the spirit and scope of the inventions.

1-107. (canceled)

108. A method for gathering genetic information, the method comprising:
a) determining the identity of at least one nucleotide in the SIRT1 locus on human chromosome 10q of a subject; and
b) creating a record which includes information about the identity of the nucleotide and information relating to an Alzheimer's Disease (AD)-related parameter of the subject, wherein the AD-related parameter is other than the genotype of a nucleotide in the 10 q AD6 region.

109. The method of claim 108 wherein the determining comprises evaluating a sample comprising human genetic material from the subject.

110. A method comprising:
a) evaluating a parameter of a SIRT1 molecule from a mammalian subject;
b) evaluating an Alzheimer's Disease (AD)-related parameter of the subject wherein the AD-related parameter is other than a parameter of a SIRT1 molecule; and
c) recording information about the SIRT1 parameter and information about the AD-related parameter, wherein the information about the parameter and information about the phenotypic trait are associated with each other in the database.

111. The method of claim 110 wherein the AD-related parameter is a phenotypic trait of the subject.

112. The method of claim 110 wherein the SIRT1 molecule is a polypeptide and the SIRT1 parameter comprises information about a SIRT1 polypeptide.

113. The method of claim 110 wherein the SIRT1 molecule is a nucleic acid and the SIRT1 parameter comprises information about identity of a nucleotide in the SIRT1 gene.

114. The method of claim 113, further comprising:
c) comparing the SIRT1 parameter to reference information, e.g., information about a corresponding nucleotide from a reference sequence.

115. The method of claim 114, wherein the reference sequence is from a reference subject who has attained old age.

116. The method of claim 114, wherein the reference subject has attained at least 85 years of age.

117. The method of claim 114, wherein the reference subject did not exhibit AD.

118. The method of claim 114, wherein the reference subject was cognitively intact.

119. The method of claim 114, wherein the reference sequence is from a reference subject that has AD.

120. The method of claim 119, wherein the reference sequence is from a reference subject that has late-onset AD (LOAD).

121. The method of claim 113, wherein the evaluating of a SIRT1 parameter includes evaluating a nucleotide position in the SIRT1 locus on both chromosomes of the subject.

122. A method for evaluating a disorder, the method comprising:
a) identifying a plurality of human individuals characterized by a disorder or having a genetic relationship with an subject characterized by the disorder;
b) comparing distribution of a plurality of genetic markers among the subjects of the first plurality to distribution of markers of the plurality of genetic markers among subjects of a second plurality of human subjects, wherein the human subjects of the second plurality have attained at least 90 years of age.

123. The method of claim 122, further comprising evaluating a measure of linkage disequilibrium.

124. The method of claim 122, wherein each subject of the first plurality is suffering or at risk for an age-associated disorder.

125. The method of claim 124, wherein the age-associated disorder is one of the following disorders: cancer; skeletal muscle atrophy; adult-onset diabetes; diabetic nephropathy, neuropathy; obesity; bone resorption; age-related macular degeneration, ALS, Bell's Palsy, atherosclerosis, cardiac diseases, chronic renal failure, type 2 diabetes, ulceration, cataract, presbiopia, glomerulonephritis, Guillan-Barre syndrome, hemorrhagic stroke, rheumatoid arthritis, inflammatory bowel disease, multiple sclerosis, SLE, Crohn's disease, osteoarthritis, Parkinson's disease, pneumonia, and urinary incontinence.

126. The method of claim 122, wherein the human subjects of the second plurality are cognitively intact at the age of 85.

127. The method of claim 122, wherein the human subjects of the second plurality are free of a symptom or diagnosis of the disorder.


SEQUENCE LISTING (3 sequences)

SEQ ID NO: 1 - DNA, 4107 bp, Homo Sapiens
  CDS (54)...(2297)
gtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgagataaccttctgttcggtgatgaaattatcactaatggttttcattcctgtgaaagtgatgaggaggatagagcctcacatgcaagctctagtgactggactccaaggccacggataggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagtaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctgttaaaaaaaaaaaaaaaaaaaa

SEQ ID NO: 2 - protein, 747 aa, Homo Sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 3 - DNA, 60001 bp, Homo sapiens
atacatgactcattatgtttctatttgacaacatttttgtatacattgagctacatctggaatttcaaggttttgagaaaaatatattcaaggtacttgataagataaaataattttatcaaaaactttgtgttgtatagaatttaacaatatttaatatcccccaaacttccttaaacaaggtgcctctaatagaagaaaattaggtacagctaatggttggtaagtcttgccaaacttctcacaaactgagaaaatgaatgactctaatgactgagtaaaaatccttttgaaagtaaggtagtttccagttatttgctttccaaaagccaaaggagaaactaatcaaattgtcagctaataggtcattaaagacaatttttgaagacagaaaattatgtgatttttggcatataactggaaagtttgaagaattgagtgacatagttgtagcaaaattccctccactttatatatgtgaaacaaggtttttcacagctgattgatgctgagcctgtctcattctagtaatgactaatattcatttacacacacttaaattaattttaaaaaattatttatttattttattattattattattattattattttgagatggagttttgctcttgtcgcccaggctggagtgcaatggcacgatctcagctcactgcaaccttcttctcccaggttcaagagattctcctgtgtcagcctcccgagtagctgggattacaggtgcccgccaccatgcccagctaattttgtactttcagtagagacagggtttcaccatgttggccagcctggtctcaaactcctcacctcaggtggtctactggcctcggcttcccaaattgctgggattacaggcgtgagccaccatgcctggccttgaaaaagtaaagtccaattcatctcattaagaaacgtacttccaataaaattttattttcttagtaattatgaatattatattaatgttattttggcaagttgtataataataataatggtgatgatagctcaatccagaagaaatatttttaacactcaggaaaaacattttttaattaaatttccatttaaagatatctttttatagtagagaagtatgatatgtgatcagtaaaagacttttcaagatttctgttaccggccaagatagaataagcctatctttttttttttttttttttgagatggagtctcactctgttgcccaggttggagtgcagcggtgcgatctcggctcactacaacctccgcctcccgggttcaagcaattctcctgcctcagcctcctgagtagctgggattacagatatgggccaccacacccagctaatttttgtatttttagtagagatggggtttctccatgttggtcaggctggtctctaactcctgacctcagatgatccacccacctccgcctcccaaagtgctgggattacaggcatgagcccctgagcctggctggatcaatttcttgaaggatacaagctatcttgtatctgcagttaaaaaattatccaaggtagacaactccagtctatcaaacatttaaggaagaaataataccagaaaacagaagagaaggaaacactttccaactccttatactttttagtctgtgttactctgataccaaaacaagagaaagatataagaaattaaaactacagatcaatgtcaccgatgatcatagatacaacaatcttccacaaaatattatcaaattgaatctgatgggagttattttggaaatgcaaggctaactcaacattcgaaaatcaattagtgggctaggcatgatggctcacgcctataatcccagcacttttggaggccaaggcaggaggactggttgagcccaggagtttgagaccagcctgggtgacaaagcgaaaccctgtccctacaaatataaaaattattattatcattattattattattgaaacagggtctcactctgtcacccaggctggagtgcagtggcatgattacacctcactgcagcctcaaccatctttcctgggttcagatcgttctcccacctcggcctcctcagtagctgggactacaggcccacaccaccacacctggctaatatttggtttggtttggtttggttttctttttgtagatacagggttttgctatgttgcccaggctattctcaaactcctgggctcaagcgatttgcctgccctggcctcacaaatttctggattataagtatgagctattgcacctggccaaaatatttaaaaattagtcaggcatggtggcacattccttgtacagctattcaggaggctgaggccagaggatcgcttgagcccaggaggttgaggctgtagcagtaagctatggtcacgccactgcactctggcctgggtgacagagtaagaccctgtcttaaaaaaacaaacaaacaaaacaaaaccaggccggacgcggtggctcatgcctgtaattccagcaatttgggaaaggctgaggtgggtggatcatgaggtgaggtgggtggatcatgaggtcaggagttcgagaccagcctggccaacatggcaaaaccctgtctctactaaaaatacaaaaattagccaggtatggtggtgggtgcctgtaattccagctacttgggaggctgaggcaggagaattgcttgaacctgggaggcagaggttgcagtgagccaagattgtgccattgcactccagcctgggtgacaagaggaagactctgtccccctacaccaaaaaaaaaaaaaaaaaagaaggtaattcaccatttcaacagcccaagaagaaaaaccatatgatcatctcaatagatgcaaaaaaaaatttagcaacatttaacacctatttataataaaaactcttagcaaactagtaataaaagaaactttcttcacctgaaaaagaacatctgttaaaaaaaaaaaactaaaactaaaagacttacagctaacgtcatactaaatagtgaaagcctgggaccaggcacagtggctcatgcctataattctaacactttgggaggccgaggtgggagcatcctttgagcccaggagttcaaaactagcctgggcaacacagtgagaccctgtctctacaaagattaaaaaattagccgggcatggtggcatgcacctatggttccagctacttgggaggctgaggcaggaggactgcttgagccaaggagttcaaggttacagtaagcacaactgcactccagcctggaaaagagagtgagacactttgtcttaaaaaagatagtgaaagaactaatgtttcctcctaatatctggaacaagataaggatatctgttgtctttactccccttcaacattgtatgaaatcataaccagtacaataaggcaagaacaactaaataaaaggcatgcagatttgaaaaaaaagaaatgaaaatgtctgtgttctcagacaacatgatagtccatgtagacaatttcaaagaatctacagaaaagcttctagaactaataagtgagtttagtaaaattggagttaatattgaaaattaattgtatttctaaatgatcagttagacaagataaatgaattttttttttttttgagacggagtcacactctgttgcccaggctggagtgcagtggcgcgatctcggctcactgcaagctccgcctcctgggttcacaccattctcctgcctcagcctcctgagtagctgggactacaggcgcccgccaccacgcccggctaattttttgtatttttagtagagatacggtttcactatgttagccaggatggtcttgatcttctgacctcgtgatccgcccacctcggcctcccaaagtgctgggattacaggtgtgagccaccgtgcccagccagaaatgagtttttaaggttgattgcatagcatggtcaccatagttaataataatgcactatatatttaaaaactggctaaaatatatttcaaatgttctcactacaaaaaaaaatgatacgtatatgaggtgatggatatgttttctttttttgtttttttttttttgagacagagtctggctctattgctgtggctggagtgcagtggcgcgatctcagctcactgcaacctccgcctcccaggttcaagcgattctcctgcctcagcctcctgagtaactgggattacaggcctgcaccaccatgcttggctaatttttgtatttttagtggagacagggtttcaccatgttgattaggctggtcttgaactccagacctcatgatccacccacctcagcctcctaaagtgctgggattacaggtgtgagccaccgcgcctggcctgtcaattagctttttcttttttattttttgagacagagtcttgctgtgtcacccaggctggagtgcagtggtgcgatcttggctctctgcagcctctgcctcttgagtagctgggactacaggtgtgtaccatcacaactggctaatttttgtatttttagtagagacggagtttcgccatgttggccagcctcgtcttgaactcctgacctcaagtgatccaccctcctcggcctcccaaagtgctgggattatagatgtgaaccaccgtgccccgccagcctcaacaattttgaagaagaagaaaaacactggaggactggcactaaatctgatttcaagatttagtataaagctacacaatcaagacagcatagagccaggcacggtggctcacgcctataatcccagcactttcggaggccgaggcacgcggatcacaaggtcaggagaataagaccatcctggccaacatggtgaaaacccgtctctactaaaaatacaaaaattacccgggcatggtggtgcacgcctgtagttccagctactcaggaggctgaggtgggagaatcgcttgaactggggaggtggaggttgcagcaagctgagatcgagctactgcactccagcatggtgacagagtgagactctgtctcagaaaaaaaaaaaaaaaaaaaaaaaaaaagacagggtagtactgtgttggtgaaaggatagacatttgcatcaatggaacagaatagagtccagaaggggacctacggaagcatgagcaattgattttttttttttttttgagatggagtctcacggtgtcacccaggctggagtgcagtggtgcgatctcggctcactgcaacctcctcctcccaggttcaagcgattctcctgcctcagacttcctagtagctgggactacaagtgccaccacacccagctaatttttgtatttttagtagagacggggtttcaccatattggccaagctgatcccgagctcctgacctcgtgttccaccagcctaggcctcccaaagggctgggattacaggcgtgagccacagcgcccgacctaaaattacaaaaattatccagatgtggtgatgggtgcctgtactcccagctactcggggggctgaggcaggagaatcacttgaaccagggaggcagaggttgcagtgagccaagttcgcgccactgcactgcagcctgggctatgaagccagactctgtctcaaaaaaaagaaaaataaaattttcagaaggaaaaccaaagaaaatatttggaaccttgggttaggtagagttctacacataatatcaacagtaaaattcataaaaaaaaaataaatggtaagctgatcttcaccaatataaaaaacttctgcctgggcgcagtggctcacacctgtaatcccagcactttgggaggccaaggcaggcagatcacctgaggtcaggagttcgagaccagcctagccaacatggcgaaattccatatctactaaaaccacaaaaattagccaggtgtggtggcgggcagctgtaatcccagctacccaggaggctgagactggagaatcccctgaacccaggaggcggaggttgcatttagccgagatcacgccactgcactgcagcctgggcaacagagcgagattctgtctcaaaacaacaacaatatagaaaacttctgctctgcaaagaacatgtttaagagaatgaaagataagctgcagtctgggagaaatatttgcaagtcaccaatctgacaaaggactttttctagaatatatagaaaacataagaccgggcgcggtggctcacgcctgtaatcccaacactttgggaggctgaggcgggcagatcacgaggtcaggagatccagaccatcctggctaacacggtaaaaccccgtctctactaaaaatacaaaaaaattagccgggtgtggtggcacgtgcctgtagtcccagctacttgagaggctgaggcaggagaatggcgttaacccgggaggcgaaggttgcagtgagccgagatcgcgccactgcccttcagcctgggtgacagaacgagactctgtctcaaaaaaaaaaaaaaaagaaaagaaaacatgaaacacaacaatatgaaaacagctctcggctttcggctcggaggaggccaaggtgcaactttcttccgtcgttccgaatccgggttcatcccacaccagccgcctccaccatgccaccgaagttcgaccccaacaagatcaaagtcgtatacctgaggtgcaccggaggtgaagtcggtgccacttctgccctggcccccaagatcggccccctgggtctgtctccaaaaaaggttggtgatgacattgccaaggcaacgggtgactggaagggcctgaggattacagtgaaactgacaattcagaacagacaagcccagattgaggtggtgccttctgcctccgccctgaccatcaaagccctcaaggagccaccaagagacggaaagaaacagagaaacattaaacacagtgggaatatcacttttgatgagatcatcaaggttgctcgacagatgcggcaccgatccttagccagagaactctctggaaccattaaagagatcctggggactgcccagtctgtgggctgtaatgttgatggccgccactctcatgacatcatagatgacatcaacagtgacaaagcaagactccatctcaacaaaaaaaaaaagagggagaaaaaaaaaagtcttgctctgtcacccaggctggagtgcagtggcacgatcttggctcactgcaacctcctcctccctggttcaagtgattctcctgcctcagcctcctgagtagctgtgattacagatgcccaccaccatgcccagctaatttttgtatttttagtagagacggagttttgccatgttggccaggctggtctcgaactcctgacctcaagtgattttgctcgcctcggcctcccaaaatgctaggattacaggccgtgcctggccagttccttactattttgatgggtgaaaactgacattttgttataggttaatgcatagttgtgtatttatgagtgaggttttacaactttcatatgtttaaaagccacttgtgtcctattttctgtgaactgaactcttcatttccaactacgctatcaatctttttcttactgattaatgagagtcctttataaaggaaatatatctttatgatactgtacagtagagttaagttcattcagtgggtggattctgctgataaaaaccaatgtgaaaatcactcctctgctgctctgcaacctagccttgataataaaagcttgccctttcattcccttgggaaccttcctcatttagagccccaatcattaccacttgccctatatggttgaaattttaggccagtgtttctcaggcatggcactgttgaaattttggctggataattatttgttgtagatggctgttttctagatttctagaacgtttagcaacacccttggcttctatctgctggatgccagcagtacatccttctcccaggtgtgacaactagaaaagtctttgaaatgtcaaatgtcctctaggggatggagggcaaaatctcccagttgagaaccactgatctaggtttatatactgctggtggaaggcaggctgcatgactaaactgcctatttgttatagtttggaaatttcacacagctccccaaatggtgtttgataaagtgcagaatcttaagttctgttggaaagtacaacatttgtcttttaacctcgtcaaaaagacaaaacaggcagaaggatgccttgaggtcaggagtttgaggccaacctgggcaacatagcaagaccccatctctattaaaaatttaaaaattagccaggtatggtggtacatggctgtagaccagctacttaggaggctgaagtgaggattgcttgaacccaggagtttgaggctgcagcagcaaactatgattgtgccactgcactccagcctaggcaacatagtaagatcctgtttctgaaaaaacaacatattttaaaccatgaaagagtagcatctgttaaatgtgtaggtatgcctaggaaatctaaaatgaagagacgtaggacaataaagtcagccaactgctcaagcctcaaagagtgggagggtcaccagagctcaagggctagcgctcagcttgcagttacaaaaaaagttactctttttcctttaaatttctgaaaaaagttctcattttaataatgtgaaattttagcaagaaatagttattaatgtgtcctcaaccctcccatctatgtccaggcccctcaatcagggatgttaaaatgttaacattttgatatgtaacttgccatactttttctaagcctctacagccttttttttttttttcttttgagacagagtcttgctctgtcacccaggctggagtgcagtggcgcgatcttggctcactgcaacctccatctcccaggttcaagcgattctcttccttcagcctccctagtagctgggactacaggcatgcgccaccatgcccggctaatttttgtatttttagcagagacagggtttcactatgttggccaggctggtctcagactcctgacctcgtgatccacccacctcaggctcccaaagtgctaggattacaggcatgagccaccgcgcccagccttatagccgtatttatatgtagatattttttctttttctttctttcttttttttttttcaagacagggtttcactcctgtcgccaaggctggagtgcaatggtgcgacctcagctcactgcaacctctgccttctgggctcaggcagttctcttcccttagattctcaagtagctgggactaccagtgcgcaccaccacgcctggctaatttttttttttttttttgagatggagtctccctctgtcgcccaggctagactgcaatggcatgatcttggctcactgcaacctccacctcccgggttcaagcgattctcctgcctcagcttcctgagtagctgggattacaggcatgcaccaccatgcccagctaatttttgtatttttagtagagatggggtttcaccgtgttggtcaggctggtgtcgaactctcgaccttgcaatccgcctgcttctgcctcccaaagttgtgggattaaaggcgtgagccaccgtgcccagcccacacctggctaatttttgtactttttatggagatggggtttcctcatgttgcccaggctggtctcaaactcctgggctcaagtagtctccctgccttggcctcccaaagtgctaggatcacaggtgtgagacaccgcacctgcccttgtatgtagatttgtatatagattttgacctaggcctggcgccatgtctcacgcctttaatcccagcactttccgaggccaatgcgggtgaatcatctgaggtcgggagttcaagatcagcctggccaaaatggtgaaactctgtctctactaaaaatacaaaaagtagccgggcatggtaacgcatgccggtaatcccagctactacccaggaggctgaggtgggagaatcgcttgaacttgggaggcagaggttgcagtgagccgagatcgcgccactgcactccagcctgggtgacagcaggagacttggtctcaaaaacaagaaaaaagaaaaaaaaaaaaaaagacattgacctaaaaggaagaagctgaggcacaaaatataatttaaagagtttaactgagccaaagtgaagatagctgcctggaagattcagacccaagtaatcttggatatgagcttcactcagctccttatccattacaagaaggttttttaagtgaaaaagtgggcggggagtgagccaataacaaaggtatctgtcagaaattctcattggtttacagaaaaagcaatgattagtgattggctatacattgttaagttatagggtgcagtttatagtgtctggtgtggcattattggatgaatttttttttcttttcactcttatgatatgctgaagcattgggtgaatttatagctacttgtggcaagcagtttcaagagacgaatacacaaagtggggagtaagttctgtcacccaggctggagtacagtggcgcaatctcggctcactgcatcctggatctccccagggtcaggtgatcctcccaccgcagacttccaagtagctgggactacaggcatgggccataacacctggctaatttttgtattttttgtagagacggggttttgccatgttggccaggatggtcttgaacttttgacctcgagcaatctcccgcttcagcctcccaaagtgttgggattacagctgtgagccactgtgcccagtctgctgtctcgtttttatgtctctttgggcctaataatttaaaatgactcacattcatcagataaaaatacttttcttttctcataggcatatagaggtttaaaaaataataataattggccaggcacagtgactcacacctataatgccagcactttgggaggccaaggcgggtggatcacgaggtcaggagtttgagacccgcctggccaacacggcgaaaccctgtctctactaaaaatatgaaaattagccgggcatggtggcacacacctgtagtcccagctattggggaggctgaggtgggagaatcgcttgaacccaggaggtggaggttgcagtgagccaagattgtgccactgcactccagcctgggcaacagagagagactccatctcaaaaaaaatttaattaataaaataaaataataataaagggatatttattaaataagaaaggacaataatgcacacattactgtgcaacttgcttttttcactcacagtgtttcacacacactcctcaacaacgacagttacaaacccttttattttttcattttttatttttttattttttttgagacagagtctcgctctgttgcccaggatggagtgcagtggcgcgatcttgggtcactgcaacctccgcctcccgggttcaagcaattctcctgcctcagcctcccgagtagctgggactacaggagcctaccaccacgcccggctaatttttttgtatttttaatagagacggggtttcactgtgttagtcaggatggtcttgatctcctgacctcgtgatccacccgcctcagcctcccgaagtgctgggattacaggtgtgagccactgcccccggcctaatttttgtatttttagtagagacagggtttctccatgttgggcaggctggtctcaaactctgacctcaagtgatcgacctgcttctgcctcccaaaatgctcggattatatacgtataatttaaataaatgtttccagattactttccaacattatgtcaacaatgtatcagtttctccatattctgcttagcaacagacagtgacaataaaaactttttttttccttttgcctatttgaggggtgaaaagtaataccttggttttttgttgttgttgttgtttgtttttttttttggtgagatggaatctcactctgtctcatgatcttgcgatcttggctcactgcagcctctgcttcctgtgttcagcgattgccttgcctcagcctcctgagtagctgggatcacagacctgtgccaccacacctggctaatttttagtagagacagggtttcacaatattggccaggctggtctcaaactgttgacctcctgcctcagcctcccaaagtgctgggattacaggtgtgagccaccactcccggtcatgttttaatgttattttattttatttttgagacggagtctcactctgtcacctaggctggagtgcagtggcgcaatctcggctcactgcaacctccacctcccaggtccaagtgattctcgttcctcagcctcccgagtagctgagattacagacgtgcaccaccatgcctggctaattatcgtatatttttagtagagatggggtttcaccattttggccaggctggtttcgaactcctgacgtcaagtgatctgcccacattggcctcccaaagtactagcattacaggcatgactgaccgcacccggatgttactttatttattttttttgagatggagtctcactctgtcgcccaggctggagtgcagtgcgtgatcttggctcactgcaagctctgcctcctgggtttccgccattcttctgcctcagcctcccgagtaactgggactacaggcgcccaccaccacgcctggctaatttttttgtatttttagtggagacggggtttcaccatgttagccaggatggtctcaatctcctgacctcatgatctgcccgcctcagcctcccaaagtgctgggattacaggcgtgagccactgcgcccagccgtattttatttttttgagttagggtcttgctgtgttgcccaggttggagtgcagtggctagaccgtggctcactgcagcctcgaccttcaggctcaaacagtcctcctgcctcagcctcccaagtagctcggactacaggcgcaaagcaggatgctggactaatttttaaaggttttggagagatgaggtctcactgtgtggcccaggctgatctcaaattcttggtctcaagccatccttccacctcagcttcctaaagtgctgggattataggtatgagccattgtacttggctaggacattttttcactacgtatccctgactactaataggtttgagcatattttaaatttttatattgtcaaatatttttatcatttaaaaaatggctcctgccttgctaaaaagttctttcttgatccaaagttataaatattctaaaatgtcatatttttatttttgtattttttcatttagatcttcaatccatctggaatttttaattttttatatttttgagatgaagttttgctcttgttgccctggctggagtgcaatggtgcaatctcggctcactgcaatctccgcctcccaggttcaagcaattctcctccctcagcctcccaagtagttgggattacaggaatgtgccaccacacctggctaatttattttttagtagagtcagtagtagtttgtccatgttggccaggctggtcttgaactcccgacttcaggtgatctgccccccctcagcctcccaaagtgctgggattacaggagtgagccaccgcgcctggctggaatttattttcgtatattgtgtgaagtagggatttccatttgtgtttttcagttttgacaataacattttattagtacaaaagagttgatactaactcccttactaattgtagtcacatgagactgatatcagagctattaggccaatttatttttttatttattcattttttatttattcttatttttattttttttgcgatggagtctcactctgtcgcccaggccagagtgcagtggcgcgatcccggctcactgcaagctccgcctcctgggttcacgccattctcctgcctcaacctcccgagtagctgggactacaggcgcccgccaccacacccggctaattttttgtatttttagtagacacggggtttcaccgtgttagccaggatggtcttgatctcctgacctcgtgatccacccgcctcgacctcccaaagtgctgggattacaggcgtcagccaccgtgcttggcaggccaatttattaacttaggagaatagaactactaagtcagccagaatattaatgttcttttttttttttttttttttttgagacagagtttcactcttgttgcccaggctggagtgcaatggcactgtctcaggtcactgcaacctccacctcctgagttcaagcgattctcccgtctcagcctcccgagtagctgggattacaggcgcatgccaccactctgggctaatttttgtatttttagtggagacggggttacatcatactggtcaggctggtctcgaactcctgacatcaggtgatccgcccgccttggcctcccaaagtgctgggatttataggcgtgagccacagcgcctagccttaatgttagttcttaaccagtggtagaccagaagtctaattgggattttttgtttttgtgtttgttgtaatacaatttctgtttctcatttatcctacctgaaaagcatactgcatctctgacctctcagcatactaacaataatattgatcacatagcatttcttgtagttcataaacttcacgtgtcaataatatactataaatataaatgaattaatgaacaacaggatgctcataagcttacagacatctttttctcaaaaaagctgggaattttgtttctgttttattgggatactgactctcaacatttcatatattgcattccaccaacgtagctgagagtcaatttatgaaatattttgtagtgtaagacagaaagtggggaggaccaagtatgtcaaccactaggagtgtggtgcctagtcaggaattgggaggagtgtagcaagaaaggaaggacaacaggatttggtcattgattggtcagatggatttcagagggattggtatgaaggaacgcttcaaagattttttttttaatttaagttccaggatacaggtgcagaatgtgtaggtttgttacatacttataggtgagccatggtggtttgctgcaccaatcaacccctcatctaggttttatttatatttttttttgagacggagtttcgctcttgctgcccaggctggtgtgcaatggtgcgatctcggctcaccgcaacctctgcctgctgggttcaagcgattctcctgcctcagccttctgagtagctgggattacaggcatgtgccaccacgcccggctaattttgtatttttagtagagacggggtttctccatgttggtcaggctggtctcgaacttccgacttcaggtgatctgtccgcctcggcctcccaaagtgctgggattacaggcagcagccaccgcgcccagccaggttttaagtcccgcatgcattagctatttgtcctaatgctctccctccccttgacccccacgcaaccaaagatggttttaaggcttctaggactggagatgattactttcagaaatgaatagtggtgaccagatgaaataagacttttggatgtagataggaccttagcgatatatttccagctttttattttttattttaaggatgaagaaacagctttaagttggttgcctaaagtcacgcaggtaattgatgcagtatggccagaacccatactaggcttaagggacagtgtaccataaactcgtagaggtcctttaaacagatagaaacgctgtgctccaggcagatgccataacaaacactggctctagatctaccatgggttatatgggtccttagttaagattagatatggagtcacagtgtgccagaatttcagggagagaggaaagtggaagggctttccactaagccttttgaactactaggtacccctcgttttacatctggttatctcatttaaatctatgacgttttaaaatacttattaccatttaagacatgagaaaaattaagtttagaaacggctagatagctcacgctagaaaggaaggactccaaattttaaccaagggcagatgtgcatggaggccaagtcatttccttcccatgctctcatactgacccaacaaacccattctgcacgtgagaaaactgaggcccggaggagggaattcacacacgtttgaagccaagctggggccagaaagtagatcggctgatctccaaacctccacgtcaaaggtcttcccaggaggacatatgccttcaaggattttacaatgtataccaccctacaagtgatgggagagaggggaaaaaagcaaccgactaaggagaaaagcaaggagcagaaaaaggagcaaaagaggagctgtcagaacggtgtgaggagagtgggaaaggagccgcctccttttgcctctcttcctacttattaacaaaacagaacgactatccaacgtatttcagggagctaagtcttagccagcttcagctgtgttttaacccttagctaaatatagacaaggctaaggcaggccaggtgtacacttcaggaagacgtggaaattcccagggcggaccaaaacttgagctgttccggcggtagtgatttgaggtcagtttgaaagagaagttgagaaagcggccgaggggcgaatttggctgcactacacgctcgccacaaagaggaagggccgccggccgccggggccgagtgcgcttccagcccaggcggagcggtagacgcaacagcctccgcccgccacgtgacccgtagtgttgtggtctggcccgcgtgggtggcgggagcgccgagagggcgggggcggcgatggggcgggtcacgtgatggggtttaaatctcccgcagccggagccgcgggggcgccagtgccgcgcgtcgagcgggagcagaggaggcgagggaggagggccagagaggcagttggaagatggcggacgaggcggccctcgcccttcagcccggcggctccccctcggcggcgggggccgacagggaggccgcgtcgtcccccgccggggagccgctccgcaagaggccgcggagagatggtcccggcctcgagcggagcccgggcgagcccggtggggcggccccagagcgtgaggtgccggcggcggccaggggctgcccgggtgcggcggcggcggcgctgtggcgggaggcggaggcagaggcggcggcggcaggcggggagcaagaggcccaggcgactgcggcggctggggaaggagacaatgggccgggcctgcagggcccatctcgggagccaccgctggccgacaacttgtacgacgaagacgacgacgacgagggcgaggaggaggaagaggcggcggcggcggcgattgggtaccgaggtgcgcagggtgcgggcggccggaactgcgcatctcctcctccctctccccgggctcctactggcctgaggttgagggcggctgggggctcggggcaggctccgcggcgttcccctccccaccccggccctccgttcagccgcgctcctccggggctgcggttcctactgcgcgagctgccagtggattcgctcttttcctccgtccgtggcccgcctgggcggccttgttctttccgcagcagccaggtcgggagactctcgcagtcgctttaaaataagtttctctccccctcttactcttttagcatattgcttttgttagagctttttttttctttttctttattttttattttcattgcttttctcctctaccccccagcacacttaagtctgcaactttttggctgtcccgttggtctgcgttttcaattgctttttgcaaacttgacacctgtgcagtttgctttttttggtctatatttttatgactgaaattacaatttttgaaagaggaaagctgtttctatagtcacagcttgcaaaatttaaagaacgtggctgttcctgaagacattaccattctgtaatgcagttaggagtgggtatgggagagagagtacttttagagctgttcatattacttttattctaatcttgctcttgtgcggtaatggtgtttgggctaaacatgctttttttgtatgtatgtagtattgagacacaaacttaacacttctagcataatgaagcagatatgttgcagttgtcatgttcttgaaggtttctttttttttttttttttttttttgagacggagtctcgctctgtcgctaggctggagtgcagtggcgcgatctcggctcagtgcaacctccgcctcccgcgttcaaccgattctccttcagcctgccgagtagctgggactacagacacccgccaccacgcccagctaatttttgtgtttttagcagagacggggtttcaccatgttggccaggatggtcttgatttcttgacttcgtgatccgtccgcctcggccgcccaaagtgctgggattacaggcgtgagccaccgcgcccggtcaggtttcttatttttgatctgaggagtttctatccttgataggtgttcatctttatcaatatttgggtcaggccgggcgcggtggctctttcgtgtaatcctagcactttgggaggccaaagcgggaggatctcttgagcctaggagtttcaggccagcctgggcaacatagcgagacctcctctctactaaaagtaaaaaaagcgggacgtggtgtcgcacacctttagtcccaggtactagagttcgaggttgcagtgagctgtgatcctgccactgcactccagggtgggcaacagagtgagaccccgtctcaaaaaaaaaaaaaaaaaaaagtaatatgtgtttttagggactgatttagtaaagagaccctgatttcaagcaacacagttgagtactaatttattatttacctaataataattgtgttattaatactgtttatttaatagaggaaacaatgcagaggtacttgtagtattgtgtaagtttaacgtgcgcattgccaaggttatatttgaatgacgtttttatgtaaatgtccatttattacacgtttcacgaacgctattgtttgtgattgtatgatacattctagctagattattatcttttctttttttttttccttttctttttttttaaaaggtggagtttcgctcttgttacccaagctggagtgcaatggcgtgatctcggctcaccgcaacctccgcttcccgggttcaagccattctcgtgcctcagcctcctgagtagttgggattacaggcatgcgccaccatgcccggctaatttttgtattattagtagagacgagatttctccatgttggtcaggctggtctcgaattctcaacctcaggtgatccgcctgcctcggcctcccaaagctctggaattagaggcgtgagccaccgctcccggctatcttttctgttttttaagtagacttaatttatttttagattttggctgaaactgagtgagcagaattcctaaagatgaagaaactgttccaatgaacagtgcaagctgaccccataggcatgctttacacacgccgtgtaaatgttaaatgctgtactcgatgaaaatgattgctctataaccgttcatacattttaggtgcatgttgttttgatagccttgactgacttggtttcttttgcagataaccttctgttcggtgatgaaattatcactaatggttttcattcctgtgaaagtgatgaggaggatagagcctcacatgcaagctctagtgactggactccaaggccacggataggtatggctcagagctgttaattttagagagtaaatgtacggtttttggtttgtttgttttgagacagagtttcgctcttgttgcggaggctggagtgcaatggcgcgatctcggctcaccgtaccctccgccttccaggttcaagcgattctcctgcctcagcctccccagcagctgggattacatgcatatgccactacgcccggctaattttgaatttttagtagagacggggtttctccatgttggtcaggctggtcttgaactcccgacctcaggtgatccgtccgcctgggcctcccaaagtgctgggattacaggcatgtgccaccttgccccacctcacttgtcccctttttgtgtagtgttcttcatccggttagaatatcttcctccctcacttcatattccttgttttttcactttattgtacacttaccacttcttgaaaagctctgtatcttactttttaaattgtgttgtttcctggtctagaatataagttctacgggagcaggaatttttgactgacttgtatactcctgtcttttcagtgcctaaaaaagtgcctggcacatagtttgtgctcaataaatatttaataaatatgtgaatgaaatgggaaagtatttcgatgtagaaagcattatcagttttttataccaaaggtataaccatcttgaaagtggatctttttgtgttctcactcttacaaatggcttgattatttaaacatagtgaagatcagtttctctttggtgtctgtaatttatttatttatttatttttgttttctcaagcctagggagctggtttgagcaatctcagggtaaaagaaaatgattagttctcagaacatcaacattgtatgtattatcttggaagatgggttaagatcaaattgaaaaattaccggaaaaaaattaagatggcagttatgtatagattttcatttctttttcctgtgtggttttaatgataagtagtacaggcttttaggactggtacgatttttctatgtcgtagaacccttttgtaaatacttttggtattatggtgtgtgtttctagatattaaccataagaacttaattctggttgttaattaattagactcttgaaatatatacttacagctgaagggggtcttcccaactcttcattagatcttcctaatctcattgggatatagcattaattctgagtgtaccgagaatgaaataccattaaattgcattttcttactttgcaaaaaaccctcacagaatgctaactcattacttcagaaaataaatgagattttgaattacttgataagttgactttaagccttttcccccttattgtaggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtatgtaagactagtacatggggaggtcgtatatgtattttcttatgccttttccaagtaggaaacatttttctggtttagaaggatttatccttacatgataatggatgtttgggaactgccaaaaactgagtgcagcagcagttgctaaattagttcagatttttaaaacctctccagccttaaaaaaatatttgcttctgcttatttatagcacagtgggtaaatggagttaaggcaagatttctacctacattatatcatagctcctaaaggagatgcagggaattgaaactctgcttttgttaagggatgtcagtctgatggagaaattgggtatttgttagatctttatgagaaactggaaacctaattggtattcacaagtgaaaagcttagtgttgggaagagtaacagattcctaccttccctccatattaattaagatgatgtgattgtggcgacttgacattttctatttgatctaccttagggttgcagctaattagttacctaagactattgcatagtgtgtgttttatacagagtatattacatgttagcaaccagagcaactgctgtagtaaaattttgtcctgttttctgccatttcgagttgcgaaagggtgttacataatcttagatgatcatcaggagtatgttttgctcacagataacgatgtagacaagtttgtttaacttaaattgaccttgactccaggtcaaattggagtcaactctggtaagatgatgaaacaataacttctgaaatctctatttgaaatgtcaggatttttttgtacgattagtgggcacgtctcaaagaaatgaagaattgaaatattatacaccttttttttttttcttttttttggatacagggtcttgctgtgttgccctggctggagtgcagtggtgtcatcatggctcactgcagtctcagcctctcctggcctgagtcttgctctgttgcctaggctggagtgcagtggcaggatcttggctcactgcaacctctgcctcctgggttcaagcaattctcttgcctcagcctcctgagtagctgggattacaggcgcacaccaccacacctggctaatttttgtatttttagtagagatggggtttcaccatattggccaggctggtctgaaactcctgaccttgtgatccgcctgccttggcctcccaaagtgccaggattacaggcgtgatccaccgtgcctggctatacattgttttttttaatgaaaatagagaattgttgtagtattgaggtaatagaatcctaactaaaggagcaatttctttggaattacttctctgtaatagaaattcttggctggtggagtggctcatgcatgtaattccagcactttgggaggctaaagtgggaggatcacttgagccctggagtttgagatcaccctgagcaacatagaccctgactctacaaaaaattagcctggtgtgctggtgcgtacttgtagtcccagctgctaaggaggttgaggtgggaggattgcttgagccggaaaggttaaggccataatgagccatgatggcaccactgcactccagcctgggcgtcagagcgaaactgtctaaaaagaaaaaaaacaaaaagccgggcgcggtggctcacacctgtaatcccagcactttgggaggctgaggcctgcggatcacgaggttaggagatcgagaccatcttggctaacatggtgacaccccgtctctactaaaaatacaaaaaatcagccggtcgcggtggcaggcacctgcattcccagctaatcagtaggctaaggcaggagaatggtgtgaacccaggaggcggagcttacagtgagccgagatcgcaccactgcactccagactgggcgacagtgtgagactctgtctcaaaaaaaaaaaaaacaaaaaaaaaaagaaaaaattcttccctacttaaaataatttgttgaaatggtttattttcttctcaagttgcatttgctttaatttgttggctaaatgcttaaaaggcttaaaattggcctgacttaaaaattccttatctgtgttaaagatggaatatgaggtatgtttaattttactcagttatttgggagttttatttctgaaataagggtagggttgtatttttatttcttaaaagaagtttcagactaaaattttctttctcaactcttacctaattatatggtaagagagctagctagttcctataaaggtagaagatttaattttacaaattatgccatgcacattttaggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggtactatgaactcttctggttgtttctttggccttctctcatgaaaaagtattttgttcacatacagccaccttaaggttatcgttcattgtttagtaaagtgaatgctgctactgtggcggagtaagatcactcattatggctagaattcctttattcctagaggaggactattatctacttcattttaggagtgagcttattttcaaagagatagttcatatttttaaaatttgcactgcagcgatggtcgttattctgcctgggcttttttaagaggtttgcacaccatataaaagtaacataacttgtgatttttaatattttattagagattgtaaaggttataacatcactttggtgtttcgtagtcaagttttaacataaggatgtgcctgaaaaatcatttgtaattagagaatgggaagattcttgggttgcatttttgtcagcaaattgcagaggatcattattctgctctagagttgcaccgtccagttcagaagccactagccacatgtggctgttgagtacttgaaatgtattgatatgtgcaataagtgtagaagacatattggattttagagatccagtatggaaaatacaaagtatttcattagttttattcatcaaatattaaacaaatattggtttttatatggaaaaaatacttaaaattaattttgaattcttttgaaatacttttgatattgggttaaataaaatgtattttgggctgtgcgtgttgcctcatgcctgtaatttcagcactttgggaggccaaggtgggaggatcagttgagcccaggagtttgagaccagcctgagcaacataatgagactccatctctacaaaataaataataaaatttgctgagcatggtggtgtgcacctgtagacccatccactcagtagtctgaggtgggaagatcccttgagctcaggagctcaagactgagtgaaccatgatctgccattgcactccaccctgggcaacagagcaagagtgtttccaaaatatatgtatgttttttgaagtttactttttttttttttctttttttgagaagtagtcttcctttgtcccccaggctggagtgcaatggcatgatcttggctcatagcaacctctgcctcccaggttcaagtgattctcctgcctcagcatcctgagtagctgggattacaggcgcccgctaccatgcccagctaatttctgtatttttagtagagacagggtttcaccaggttggccaggctggtgttgaactcctgacttcaggtgatccaccctcctcggcctcccaaggctctgggattataggcgtgagccaccgcgcctggccagttttcaccttaatgtggttactaaacaatttaaagttatatgtatgtctcacattatgtacctattgtacagctctgctttagcatagaaagtttaataatgcattacaccattctcaagagattgagtcattagaaaactgttttctttcctgtatttcagtcttttggaaagaaattacaaaatttgttattgttaaacttggaggtatatatatttgaaagagtcattttattttattttactttaagtgccgggatacatgtgcagaatgcgcagatttgttaacataggtatacatgtgccatggtggtttgctgcatctatcaacctgtcatctaggttgtaagccccacattcattaggtatttgtcctaatgcttttccaccccttgccccccacctcctgacaggcctctgtgtgtggtgttccccaccctgtgtccatgtgttcttattgttcaactctcacttatgagtgaggacatgtggtgtttggttttctgttcttgtgttagtttgctgaggatgatggcttccagcgaaggagtcttgtattagtggctttttccccacctaatcgttagaagttgtgaatagggacttctttaatgaatccagaagttaatgaacctagcttttttttttttttttttggagacggagtctggctctgtttcccaggctggagtgtagtggtgtgatctctgctcaccatacaacctccacttcccgagttcaagagattctcctgcctcagccttccgagttgttgggactacaggcgcgtgctgccatgcctggctaatttttgtatttttggtagagacggggattcactatgttggccaggttggtcttgaactcctgaccttgtggtccgcccatctcagcctcccaaagtgctgggattacaggtgcgaggcaccgcgccccgcgccctgccgatgaacctaaattttaactaaacagtggccttatctactttcagaccatgtggtatatttacatgactacaggagaagctttgctaattcagaataaattatgttacttaaattggcgactgtctttaaaaaaaaagtgattttttggatgcagtagctcgtgcctataatcccagtgctttgggaagctgagataggaagatctcttgaactccggagtttgagaccagcctgcgcaacacagtgagaccctaatcactacacccctccaccccatgtaacttttgttttatccaggttgtggtatattaaatgggcattagtgtaaagtgggaaaattatattaattcttgaatatgattactaaactgaatttgaaaagttttcaggctatcaagagaatttttaacttaaaacttatataatttgaactttttacttcatatatccgtaatgatgatggtcatctatatctagcttttagagcagacaactggttcatacctggattaaataaataatgtaaagttattttttgttaattatggattagtgaagatttactgttttaacctactcctgctagtggcactactgcatggttttgaagaccagtgaagtatgacttaaaggtttcttgaattggagctaggacactggcatttataaaatctccacgtagcgcagacattgacactatcagaagaccagcaagtaactagaagttactttgatcttaaatcaactacaaaaaacttgactcactagttatggagaatacattttttattgttaaactgaaaaaaaattcagtcacttataaggtgtagcctcttctaatcctgtttatataaatttatttattattattattttttgagatggagtcttgctctgttgccaggctgtagtgcattggcgcgatctcggctcactgcatcctccacctcccgggttcaagcaattctcttgcctcagcctcccaagtagctgggactacaggcacgtgccatcacgatcagcacttttagtagagacggggtttcaccatgttggcaaggacgtgtctcgtcgtgacctcgtgatccgcccgtttcagcctcccaaagtgttgggattacaggtgtgagccacaatgtccagctgataaatttaattttgcttttctgagttttcatttatggtaatggttaaatcagctaggctttacagttgttactcacatataattctttgtccatcctttaattctcacattgggaactgactaaaaaaagaaagcttccagtctgggcacagtggctcacacctgtaatcccagcactttgggaggccgaggtgggtggatcacctgaggtcaggagttcaagaccagcctggccaacacagcaataccccgtctctaataaaaaatacaaaaatcagccaggcttgggtggcgggcgcctgtcgtcccagctacgcaggaggctgagacggggagaattgcttgaagccaggaggtggaggttgcagtgagccgaaagcgcttaaggagaaataagtaaggacaaagagtgcaggatagtatcctgacacggaggatggggagggtatgtaaatattgtcgatattttactgaatttctaaatattacaagaatagaatattcataggataacctaagcttttctgtgagtcagtgtactaggcagaaagttcactaaagtggtttatgatttttgctacttatgagaaaacattaaaaatgaagccatgaaaaggaaatattggtgaaatttagatacttcagaaggaaagagagactttcttttttttttggaaagaatttctataccatctattatatcattgattgtatgtgattattgagaattaacttgttttttttttttttgttttttttttttgagacagttttgctcttgtcacccaggctggagtgccgtggtgagatctcggctcactacaaccttcacctcccgagttcaagcaattctgctgcctcagtctactgagtagctgggattacaggtgcccatcaccacgcccagctaatttttctaattttagtagagacgggatttcaccatgttggtcaggctggtctcgaactcctgacctcaggtgatccgcccgcctaggccaaagtgctgggattacaggcgtgagtctcccacgcccagcccttaaatttcatttagactggaaatttcaagtaatggagaggtgtgttaaggaagttattgctctgttagagcagtttcttaaatttggcactattgacattttgggctggatttttctttttgtttctgagatagtttcactctgtcgagccatcacagctcgttgcagccttgaactcctgggctccttgaacaggaggctcctgcctgagcctcctgagtagctgagaccataggcacgtaccatatacctagctaaatgtgtttttgactttcttttttctttgtagagtcaagttctcactatgttgtccaggctggtcttgaaatcctgggttccagcaattctcctgcttagccttacagagtgtcgggattgtaggcataggccactgcacccggccttgggctgatatttatttgctatggggaactttcctgtgcgttgtaggatgtttggcaatgtccctggccggccaggtgcagcggctcacacctgtaatcccagcactttgggaggctgaggtgggcggatcacttgaggctgggagttcagaagagaccagccaacatggtgaaaccctgtctctactaaaaatacaaaaattagccgggtgtggtgacacacatgagaatcacttgaactcaggaggctgagtttgccgtgagctgagatcgcgctgctgtactccagcctgggtgacaaagcaagaatctgtctcaaaaaaaaaaaaaaaaaaaaaaaaaattcgctggtctccacctacgtttgtatccccaccccaagtcctgacaataaaaaatgtcttcaaaatgttccttgcaaaattgctcattgaggctaactgaggtggctcacaactttaatcccagccctttggaaggctgaggctggcagatcacttaagcccaggtgactggccctggccaacttggcaaaaccctgtctctactaagaatacaaaaaatagccagacgtggtggtgcacacctgtaattccagctactcgggaggctgaggcagcagaatcacttgaacctgggaggcggaggttgcagtgagccgagatcgcaccactctactccagcctgattgacagagtgagactccatctcaaaaaaaaaagaaattactctctgagcaccagtgtgttacagtgtgcttagctgtggtaccactttggattctttcacacaagaacagttaaggccagttttaaagctgtggggccatttctgaactgtattttgtttttttttttttggagatgaagtttcactcttgttgctcaggctggagtgcgatggtgtgatctcggctcacttgcaacctccgcctcccaggttcaagcaatctcctgccttagcctcttgagtagacttgattatgggatcttggctcactgcaacctctgcttcccaggttcaaacagttctgccttagcctgctgagtagctgggattacaggtgcccgccaccatgcttggctaattttgtatttttagtagagacggggtttcaccatattggccaggctggtctcgaactcttgaccttgtgattcacccacctctgcctcccaaagtgctggtattacaggcatgagccactgtgcctagccaattttttgaatttttagtagagatggggtttcatcatgttggccacgctggtctcgaactcctgacctcaggtgatccacccaccttggcctcctaaagttctgggattacaggcgtgagccacttcgcccagcctgcactgtgttcttatcctgcataatgacttaaattgataataagttgtggtcatggttctcagaaacctaaaataattcactgatgttaataaaaatgagtggttgtatttagagtcaaggcgaaactgaattaggatgtataaaataactttttttttttttttttttttttaagtaggactgttgttcggctgggcgtggtggctcacgcctgtaatcccagcactttgggaggccacagcgggcggtcaaaagtttgagaccagcctgaccaacatggtgaaaccctgtctctactaaaaatacaaaaattagatggatgtggtggcgcatgctgtaatcccagctactcaggaggctgaagcaggagaatcacttgaacccaggaggcggaggttgcagtgagctgagatggcaccactgcactccagcctgggcgagagagtgagactctgtctcaaaaaaaaaaaaaaagaaaagaaaaaaaaagaaaagggactatactatgttcgaagagtgtagtttctgtcccatactgaattcactgttctttccctagtgattaaaatacaagaaaaggtagagggagaaacacgaactgaattttatgaacttaattccaagaatgtatttttacatttgttcaaaaaattattactttattgataatatgcaaacataccagattatgtattaatagttaaaaatttgagttctagaacaagcttattaaaaatcaacgtcagtaaaaagaaaatagatggtagtcacagtgccattcatctgaaaataatgttttaatactcaacatgtatatggattatatatctatacgtttatagtattgtagctggatttatgttacatttttagcataaaatttggcttttgctgggcccagtggcttgcgactgtagtcccagcaactcaggagggtaaggtgggaggattgcttgaaccaaagagtttgaggctgcagtgagctgtgacctcacctctgcacaccagcctgagtgacagagcaagacccatccctgaaaagaaaaaacttggctgttttactttgctttcaatttgtagtatattctacttaatataataaacttactttctgtgaggtgcttgaatactttttaatggttatttatgaagtggtgaactgtaattctgtatttgtacatttggatgtacaaatagtgttgctacagttgtccttttttggtatgtctccagtgttcttaggataaattcctgaaagtgaatttcagttttgaatttcagagaaacatgtcaaagttcttctttaagcctttaagtctcctgatatgttttgtcgtattgcagcttggaaaggttgaaataaaatcctctattagggctgggtgcagtggctcatgcctgtaatccgagcactttgggatttagctgatttggctgatttatcatgccactgccacctgtatccacccaaaatccacaaaaaccccaggatacattcttagcattaaaaaaaaagttatataaatatatataattatatatatatttgagacagggcctgtctctgatacccagacgtgtgcagtatctttcaaaaatcatatatctatatatagtttgccccttccttcagtctttgctgcccctaatatctgtccttaatggttaccatggtgaaactcttggaaatcattcagggggaaaaaaaatttgtgttgggataggggagggatgtcagcataagtttttaatacttgataaatgattgccaacagaatcaaaaacaccttgttttctatcttttttcaccatataatattcttgagaataatttcgtaacagcacttaacagatataccttatttttcaaagaaggctgtgtagcattctattttaaagttgtttcacagtatatgagtctcctattgataggtatctgcatttttgtctttttgatctagggttgtttttaaggcagtttaaaaactatagtaagtaaaagaggccaggtgcggtggctcacacctgtaaacccagcactttgggaggccgaggcgggtggatcacttgaggtaggagtttgagaccagcctggcaaacatggtgaaacccttctctactaaaaatacaaaaaattagccggccgtggtggcaagcgcctgtagtcccagctgttcggaaggctgagggaggagaatcgcttgaactcgggaggcggagattgcagtgagccaataatatgctgctgtactccagcctgggcaacagagcgagactctatctcaaaaataaatacaattttaaataaaattataaaaataaagcagcacccagctttattttttatttatttatttattttttgagacagagtcgcattctgtcgctcaagttggagtgcagtggtgccatcttggctcactgcaacctctgcctcccgggttcaagtgattctcccgcttcagcctcccaggtagctgagattacaggtgtgtgccaccacgcccagctaatttgtatttttagtagagatgggggtttcaccatgtttgccaggctggtcttgaactcctaacctcaggtgatccgcccaccttggcctcccaaagtgctgggtttataggtgtgagccgccgcgcttggctcaacatcttattgtttgagacggggtctccttctgttatccaagctggagtgtagtagtgtgatcttacagctcactgcagcctccaactcttgggctcaaatgattctcctgtctcagcctcccaagtagctagtactacaggtgtgcaccaccatgccctgctgtttctttgttttttttatatggacagtcttgctatgttgcccaggctggtctcaaactcctgagctcgagtgattctgctgcctcagctactcaaagtgctgggattgtaggcgttaactatcaggttaggcctgttctgtttttattagcgtggatgactgctggttactactggccgtttgtatatccttgaattacctctttgtttttagccatttttctccatttgggtgctttttcacagtgacttctgggaatattatgttcttttcttttgatttacctttcaaattttatttttatggatatataatagttgtacgtgtacattatatattcttgaagagttaggcttattgggattccagcctttagtagtcccatgaaggtgagcaaactgaaatgtacttactagtcttagtcatttggttcgtaatatgtaatagaaatggtgcttatcctaaaagaaaaatagaaaacttgagggtgaaggatcagaaattgaagacagtaaatactgcatagaggtttaaatgctagggtttttaggttttcttttttttttgagatagtcttgctctgacacccaggctggactgcaacctctgttccctggcctcaagcgatcctcccaccttagcctcctgactagctgtgaccacagatgtgcaccaccatgcccggctatttttttttgtatttttggtagagatggggtctcgccatgttgcccagtccagtgctggattttcatatcaaaacagggctctgctacttacttgctcttgtgaccaaaatggcaagttaggtttgttttgtatttgttttcatctgcatcggggtatatgttcacagctggatcagcaaactttctgtagtaaggtagggcctggtagtaaatatgtagtatgctttgcagactgcatagggtctctgtcatatgttctttgttttctttacagccttgaacaaattaaaaaacccagtcttcagccttgtctgtagcataataaagaggaaattaaaagtgcttatgttatacgtggtacatactaaaatttattaagtggcgactggcagtttaactaattgacattctcttgtgttaggttaatacaggggttggcgtactatggcctcagtccaaatgtggcccactccatgtttttgtataggctgtgagctaagcattaagcattaatttattacttaaatcaacaaataaagtttatgtggtgtacaacatgatgttttgatgtgtgtacattatggaatggctaactcaagctaatattcattacttcacatacttttttttggtggtgagaacatttaaaatgtactcttaataattttcaggtatacaatatttgtttttgtttttgtttttgttttgagacgaagtctcgctcttgtccccaggctggagtgcagtggcgctatcttggctcactgcaacctccgcctcccaggtacaagcgattctcctgcctcagcctcccaagtagctgggattacaggcgcctgtctccacgcccggctagttattgtatttttagtaaagacagggtttcaccatgttggccaggctggtctcaaactcctgacctcaggtgatccgcctgcctcagcctcccaaagtgctgggattacaggcgtgagccaccgcgcccggctacaatatgttcttaattatagtcagcaatagatctcctgaacttattcttcccagcgtcttcccaatcccacctccaccccctgctaataattaattttaaatgactggggaaaaaattgaaagaactgtttcatgacacttgaacattatataaaattttcatgtcagtgttcataaatacaaacttttattagaacatagccatactatttatatattgaaatacagttgaatttgtcatatgaccaatttattagtattgattaagatgttgagcagtttattcaagtgtggttttgtggaatgaaagagacaggagggattatgaggactctccttagcaggattgatgcatttcactttttttgacaggaaggaaagtttaggttctattttctcagcaactctacttaactgcctccatctgaactatatataggagggggtggatatcagcaaagattaacatcagtaaattttgatgaattctctaaaaattctgatttttaaagagggaattttggctgggcacagtggctcacacctgtaatcctaagcactttgggaggccaaggcaggtggatcacctgaggtctggagtttgagaccagcctggccaacatggtgaaaccctgtctctactaaaaacaaattagctgaatgtggtggcgcgcacctgtagtcccagctccttgggaggctgaggtgggagaattgcctaaacccaggagaggtggaggttgcagtgagccgagattgtgccattgcactccatcctgggtgacagagcgagactctcaaaaaaaaaggtgggtgtgaattttgttatgttgtgcgggctggagtgtagtgcccatccattggcacgatcatagagtactacagctccctaactcccagactcaagtgattctccctcctctgtctccccagtagcaggaactacaggcctgtgccaccatgcccagctttcttagtcttgagggactgcatttacacctatgccttttactctagggtcttttattcatattgctagaaatttggagaagaaagaaaggcataatctctgcagaaaagccattatttctgcagatagttctttttttttttgagatggagtctttctttgtcacccaggctggagtgcagtggtgtgatctaggctcactacaagctccacctcctgggttcacgccattctcctgccttagcctcccgagtagtgggactacaggcacccgccaccatgcccagctaatttttttttgtattttttagtagagacggggtttcaccatcttagccaggatggtctcgatcttctgaccttgtgatccgccgacctcggccacccaaagtgctgggattacaggcatgagccaccacatccggccaatttctgcggatagttctaagctttagttttgaccacgttggttgcctatggccaattcctggattatttgcatgtttccagacacagctatgtgaaagcagggtttgttttaatagtgttttgagcctttgattccagttctgcttccactcttggtctataaggatccctatggcagcaacagaatggcccagccctcatgattggttgcttaaaaaggctttaggatattcaaactgggtggcctgtcttgtttggaataggactaagcatgtcacactttcaccatggaagagttaatccatattctatctgtaagttgtatctccagtcattggttttcaacaagtggtcctatcttaggaggagtggtatattagaacagtccttccagagtaatttttttcattatttcagattttttagttttttttgtttgttttttttttttttttttttaaaggctctcactctgtcacccaggctggagagcagtgatacgatcatagctcactggaacctctacctccctgggctcaggtgatcctcccacctcagccttctgagtttctgggactacaggcatgaaccgtgaggcccagctaatttttgtgatttttgtgttttttgtagagtcagggtttctccatgttgcccaggctggtctcgtactcctggtctcaagtaacctgcccgcctcggccttccaaagtgctgggattagaggtgtgagccaccacacctgtactactactttagtttgaacactatatttaaatctaataattcagacatggcccatttcatacttgacagaaagtatgagtcaaagactcaaatcattcagattctcacatatgtaaaacgtacgcttggccgggtgcagtggctcacgcctgtaatcccagcactttgtgagaccaaggtgggcggatcacctgaggtcaggagttcaagaccagcctgaccaacatggagaaaccccgtctctactaaaaatacaaaattagctggggtggtggcacatgcctgtaatcccagctattcaggaagctgaggcaggagaatcacttgaacccgggaggtggaggttgtggtgagctgagatcgcgccattgcactccagcctgggcaaaaagagcgaaactctgtctcaaaaaaaaaaaaaaaagcaggcttataaaataaaacaaaaatgtcccttatgatttgtcttaaaggtagttgaattaataagagctaaatctagtgtcccatacatctttgtgtttcttactctaccctttagaaagggtgggggtggacgtggataaggcacacagttaattcagggacggtggagtcttggtacttgctctttttccctgaccttgctttggagttattttattttttaaattctgcatttggcatgctattatcacttgcttactattataggtcctattttatctgttgcctagcatattctcatagtgcctaaaatactctaaaagctatttccagagggagctttaatgagacatacaatgcttaatctctcaaatgtaaatgtactagaacaaagcattctggggaagaaaacagtggttgatttaactgaactcacttttccaaaacagtggcttttgattttctgtaaacattgactttattaagcttttcccttgctactatatgcactctctatttcttaaattgcaaagagttattcatgaaatttgtgataaatatttgaatctaaggcaacgtttttggattctttatacgaggtttttagcagcatgtgtacatgtatgtgatagctgttaacggaattcacttaagttttacttagaagcctgggtagtagaacctagaagttgcagtgggaatttttttttaatttgaaagattaaataggtaaaaagtcatctttaatctctattaaacttttttgtataatgtcagaagtgaaagttccccacatctgcttacagcctgtttgtatccttcctgacttgattgattaaatggacatctctatatacatatgcatataatttcaatttttgcttagtaacaaaaacaaaagttacacatactggtgactaatttgcttttttcacgtatgtcatgaaccattttccagggcagttcatctagatctccctcaattctttttaatggctacatattccactattatggatgtaccatatttatacaaccggttccctattgatggacatatggttgtgtctagttttttgctagcattgtgatagattagagcctgtaatgaatggtaaggaagtaagtagatgatagcactactactttgaaggaaaaagtagagaataaacttcctttgccatagtcacttactaaatggaatttaataaaaacactgtcaaaagttgggaggaccaaaattgatactttttctctgatctttttgccatgtgtatatctgaattctttgtttttaaagaagaaacagcattgaagcattatttggggggaaaaacacacacacaaaatccagcaactcagcattcatgagcaactctatactataccagtatgtgcctgtgcagtggaaggaaaacaattttggtaaggattaaaactttagctttaaacttccagcaggttgatattctaatgaatgataaatcaaaaaaaaattttaaatattcttgtattgacagtgcttttttttttaaatcaccctaccttgatatctgtaattttagtttaaactttcccatttttctttaaagaaaaaaatctgaatttgcagccaacaaaaattagatatcctaaggttgtattacttctgacttttattaaaatatcaacatttcttagagatgtttatgggccgactttgtctttttcttcaaggggccaagttcactaattgctgagttttatgcatatgacagcaaccgtccttttgtaggtgtgtgtcgcatccatctagatactttaaaatgctcatctatttcatttttaaaattatgtgtgtgggattatcagtatttttttgttaaacatatgacatctgtagtttatttcactaatgtaaattttttctaccatttgcttgatacaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttagtaaacttcagagtggttttctgtaatttattttagttttataggaagatatttcctataaagctgactgccatcgagaagtggagataaagcattatttaatcatgttatctcatttatcgataacctcagaaaagtagaaaacaaaaataataaaaacagaagtattggccttgacagttaattatagaaaacctcagatattaatttgcttttgatttcaaaagatgctgcagttgcatgcattcaattattttaaataatcttttctggctgagtgcagtggctcacgcctgtaatcctagcactttgggaagccgaggcgggcagatcccatgaagccaagagttcgagaacagcttggccaacatggtgaaatcccaactctactagaaatactaaaattagccgggcatggtggcaggcgcctgtaatcccagctactcaggaggctgaggcacgagaatcacttgaacctgggagttggaggttgcagcgagccaagattgcaccattgcgctccagcctgggagacagtgagactctgtcaaaaaaaaaaaaaaaaaagaaaaagaaattctgaatacaagagtagtattagctgttaatgaagaaatgtgacatctttagtttatgaaactaaaagaactggatagttgagatgtacaggattcagagattcagaaatgtttaaaacaagtatcaacaggccattaggtatctaaagtgtttctaagaactgccgagctaaggtaatagagttggaactgtccaactctgcaggattttagccctggaggagtgagctgttacagttttgttgaaaagagtagctttagaaggcatcattatattagtgtctcagagattgagaatcatattcattctatgtataaatatataaattcttcatcccctagattctcctgtagtatatcacaaaatctgcagtgtgttctgaggtttaaaatcaaagttaaaaacaaaaacaaaaatccttaaaccctcttaacatttgtgatgttaaactttataacgtttgtggtgtgttcaagaaacagaaatacttctttaataaagcatatatatgttgtttgtttttaggttcctttgcaacagcatcttgcctgatttgtaaatacaaagttgactgtgaagctgtacgaggagatatttttaatcaggtaatttgttgcccatattttaggaattgttcatgtctctgaagtatttcttcttttgcctcaaaatccttttttacccctttaaagtatatatggtacagaaagattcaggaagaaaatagttagcatttgggaattttggtaaaatacacgagaacctttcaataccttatatagaaaacagccctataaaggctcttcaaattatgaaattggcttcttagtatctaagccgaacataaaatgtcagatcataagcaagttgttgttgtagtagtttttctccttcctctccctttttgtgcctgtcagatttggaccttaaattagttatggtaatctaaaattatttcatacatgttcattgccaaaaaatataggaaaatttggcagaaaataaaaattatccagaaatatgtcctgtgtgcttagcaatatatggaggtaatcatgtcattacgaaagaggaagaatagtatattaaaatggagtcattggtcgggtgcagtggctcatgcttgtaatcccagcactttgggaggacgaggtgggtcgatcatttgaggtcagaagtgagaccagcctggccaacatggcaaaaccccgtctctcttaaaaatacaaaaattagcctggcatggtggcatatgcctttaatctcagctgcttgggaggctgaggcaggagaattatttgaactcgggaggtggaggttgtagtgagctgagatcatgccacttcactccaggctgggcaacagaacaagactccatctcaaataaataaaatggaatcattttatttgacaaatgtctatttttttttgaaaggatcaattcccatttagtgtatatacacacattatgtttttcctcttaatatatgatggactcttcaatgtcaagtgtttttctacatagatattataacaagagcatagtatttcattgtttcatagtatgttaataaactacccatcattgacagttgctgtttatccatagcttttctgttttgttttttttttaataattctgaaatgtattcttagaggtatggaaatgttgactatttctaacttgggcttactctttgcttctctacctcaaccaaaatctgaaaatatgtaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagtaagttggtgatggtttttggagaacatttctatatataatgtcatgggttgtgggtctgtataatagacgctagtaatcttaactctgcttctgtttgaaagagtggtgaagagctaattttagaaattgtttgtttgtttgtttattgagatggagtttccctcttgttgcccaggctggagtgcagtcgcatatcttggctcactgcaacctccgcctcccgggttcaagcgattcttctgcctcagccttctgagtagctgggatcacaggcgtccaccaacatgcctggctagtttttgtatttttagtatagaccgggtttcaccatgttggccaggctggtctcgaactcctcagctcaggtgatccgcctgtctctgccttctaaagtgctgggattacaggcatgagcccccatgcctggccagaaattctttatttttagtagagatgaggtcttgttgtgttgcccaggctggattcgaactcctgggctcaagtgatcctcctgcctcagccccttgagtagctaggattacaggcacgagcctccacatctggctgaactgtttttttaggtggcattgttcattgagactggtgaatctgacattttgatggggggtggagggttgtcaaaacgcaagtaatgttggtggctcgtgcctgtaatctcagcactttgggaggccgaggtaggtggatcgcttgagggcagcagtttgagaccagcctggccaacatggtgaaaccctgtctctactaaaaatacaaaaattagctgggcatagtggtgtgcacatgtagtcccagctacttgggaggttggggcatgagaatcgcttgaactcaggaggcagaggttgcaatgagccgagaatgtgccactgcactccagcctgggtgacagagtgggactctgtctcaaaaaatcaaaaacaaacccagaagtaatgctaaactctacttctaatttatgtgaaaaattaagacaaaggtagagttcaacttagactttttggtggaatgtgtttttaatgttgcaagggttagcatcaattaatttatgaaatggtcctttatccttatattttttgattacagttttgatctttcaaatttaattgatttcacatttaataaattcaaatgtatagtttggtagaagtgtaacataccatgtaagatggaatttgggggctcagaatgattggttcattcttgtagtaacagccatgccataacggtgatatgtcgatatagtttacccaaaagggtgtgtatataaaagtgttcacataaaatttaaatccttattactctcaggaaaatttctttgtcatatatattctcatactgtctgtttcctcctctagtttagaatcagaggaggagagagagagatttcagttgcatcagatgtgtcctttcataagtgagggaaagaggagaggaaaaatattaaatccctagccagagaattgaattagaccccacatcagcagtcaaggcagccagagtaaacagttggaagaaacatggagtcaagctcttcaattttgtgtctatcccatatctcgtggctttagcagttaactcagtcttacctaattagctgtgaaattctgttaaagtagaatacaagacaatttgcaattaaatgaaacatgaaaatttaatatacaaatctctttgttcttgagttgctcttcttaaagcaaaatcaaaggcactctgcttaaaagcttttctttttattttttagagacgggtgtcttgctgtagctcaggctagagtgcagtggtgcagtttcagctcactgcagccttcaactcctggggacaagcagtccttccacttcagcttcctgagtagttaggaccagaggcgcacaccatgcttggcaaatttttaaatttttttttgtagaaacgggattcactttgttgcccaggctggtcacaaactcctggcctcaagtgatccttcctcctctgcctctcaaagcattaggattacaggcatgagctaccatgcctggtccttaaaaacttttctttcaaaagctttctgtgggatagtatgatgtgttgcaaagataattaaaaaaagaaacaggtttataatagcgtagaatatgtttatatgactttttccctccccccctcccctccctccctcccttccgtttgtccttccttccattcatccttccttctgttagtccttccttctgtccatccttcccgtccgttcttccttccttccttcgtccatccttctgtctgtccttccttccgttcatctgtccgtccttccttcctcccaccctccctccctccctcctatcctccctccctccctcattgcccaggctggagtgcaatggcatgatctcggctcactgcagtctcccgtgttcaagcagttctcctcccaagtagttgggattagaggcatgcgctaccacacgcccggctaattttgtgtttttagtagagatggggtttcaccatgttggtcgggttggtcttgaactcctgacctcaggtgatccgcctgcctcggcctcccaaagtgctgggattacaggcgtgagccactgtacccagctcaccttctttatatcaggagctacttaagtagaacatttatgtaccaagaactcttctgtgcacttgactaactcatttaatcctcaccacagtcttgtggagaagtactattatcatccccattctgcagacaaggaaatttgaggttcagagtgggaaagtctgggaagattgctcaggggtaaccaggtgataggagcagaacttgagcgttttataaaagacacttaactgcccatctgcttgcttgatgaaatgtaatggcttggttaagtatttagtgcatgggtcttttttgggaatttggagctcaagcccttgttggatttttgcataatgtatctgttgtggttttattagcttacttcctcctccctttttctaactcttatttttcaccctattttaggttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaacacaaaaagaattggcttatttgtcagagttgccacccacacctcttcatgtttcagaagactcaagttcaccagaaagaacttcaccaccagattcttcagtgattgtcacacttttagaccaagcagctaagagtaatgatgatttagatgtgtctgaatcaaaaggttgtatggaagaaaaaccacaggaagtacaaacttctaggaatgttgaaagtattgctgaacagatggaaaatccggatttgaagaatgttggttctagtactggggagaaaaatgaaagaacttcagtggctggaacagtgagaaaatgctggcctaatagagtggcaaaggagcagattagtaggcggcttgatggtaagaaaggcagtcggaccattttgaaagtataaatgtcataacagtatttccaaaaaattagctatttcggcaggttaatcgatagggtagctttatgtagttgattctgtttagagaaactgtacagttcgtaatcagaaaggtaaatcttctggtatcttaacatgatatggagaaggaagtgtttaatagtgctctgtatgttgtgtttctctaggggatggaaaaataagaatgggttattagctggcagaaatgatgacagatttgagtgcttactgtaggtcagctgctttacatatgttacctgattttaatctgaggtaggaactattactcttcttcaggtgggaaaactcaggaccatagaggttaaatacctcatgcacagtaactactaagaagtggaagaaccagattaaaatccagtctatttatgcttagagcctgcccccttaactactatgcggtgttgtctcagatgtagaacacatctttgttttctctgaggacttagaaaaaacacgcgtgattcttctccactgaatataggaatagtttctaccctgtttagaacatgcatagtctttatcaataagtgttaactgacatgtgaaatcactttctactctttggaagccgatgttttaagtccaagtagtctgtatggtggaaattcctaacttatgttaaaaaaaagaaaaccactgtaacttagctataacagtcttatataacttagaactttaagtctcattccatttaccatacttggcgaaaactgacaaattttagacaactgtatttgagtattccagtcatttgctgtaaatacattttagttcagttgaataaacctttggcctttttcatattaggcactgtggtaagttatggagacacagtgattaatgttgtgtataattataaatgtgagtgatactcattaatactttggtataggtgctgctgaagaacaaagtactttattattgttttgaacaaaacatcacacaaaaggtagattttttttttttttttttttttttttttttgtgacggagtctcactctgtcgcccaggctggagtgcagtggcacgatctcagctcactgcaacctctgcgtcccaggttcaagagattctcctgcctcagccttctgagtagctgaaattacaggcatgcgccaccttgctcggctgattcttgtatttttagtagagacggggtttcgccatgttggtcaggctggtctcaaactcctgaccccatgatccatccacctcggcctcccaaagtgctggaattacaggcgtgagccacggcgcccagccccaaaaggtagattctacttggagattagattacagaaggctttctaaggagcaaaatatttaacaataagtagggatttaaaaagagcagacgtgttcacaggggaaactagaaaaagcatagagggatgcttacgtttgcaaatcgtggcagaagtcagaaagtagaaaaattgctactgacttagatacacagttgtctctagcgtatacaaagtctacctatgcactaagacttgcactgggaacttttcttgaatgtcattttgacagatgtgtcaaacggacaatctctttgcccaggaaacagagttaacctggaacagtcatttttttttaaatttatttattttttgagacggagttttgctcttgttgtccaggctgaagtacaatggtgtgatctcggttcacggcaacctccgcctcccgggttcaagcgattctcctgcctcactctccagagtagctgtagctgagaccacaggcgcatgccctcatgctcagctattttttttttttttttttttagtttttgtagaaacaaggtcttgccacattgcccaggctgataccaaactcctgggctcaagcagtctgccagccttggcctcccaaagtgctgggattacaggtgtaagccactgtgccctgcctgtggtgtcttgggaaactcatgagtactatgtgtctgttgtaatagagggaaataagtggttttcacagtgatttgtagtggactgtgaaattttagggattcaggtcagagttgtcacacaggttgtagtcagggtgagaactgggtcatgatgcagtatgaaaaagttcgagagccactttggagagaacttgagataggccacctaccagtgtggtaaccaggcttttgagaattcgtctgggatacggtacaataaatactacatctattatgtgtgaagagatgttaagttagggacatactgtgaattcaaggatagaaaacttttccatcagtttttagggatcctactctttcacttaaaccccaaatggccaagctaggattgatttggtgtgctgtagaaagaacttcattggtattcatggattcacattacatcttagaggagttttcaaaagcgtcttagactgtatgtgtatatacacacacattctgaagcagtaggtgggtcttggggcctgagatctcgggtgaatgtaaatttaggttcacaggtgatactgtagattcacagtgtctacagagtacactatgaatttgtggtgactacattattgacaaaatattttaggtttataatcagaaaaaagttaaaatagttaatgaagatgctttaaaagcctgtgtactttagagaagctacttaacacaaattgggtatctaatgtaggctgggctggatacttcattttcatcaaatctttttaaaataattggtgaaataacctttattgaatatggttttctacatttttcacacttccctccttcatagggttgtgaaaatttatttcatattctagatgaggaaattgaggcacagaggtacacttacaaagatacaataaatggcagaactaagatttgaacccaggactaagtgtattgcttgtatttatttaattaattaatttttaagagacagggcctcactctgttgcctaggctggcccttgaactcctgggctcaagcagtccacctgcctcagcctcctgagtagctgggactgcaggcacaccatgcctcccagttgtttttaaacactaatagtagtctttcataaggacacttataataaaggcagagctggaacccacacttcattccagactgctcagactgagttagtgttagaaaactgaaagtaacatttttattactgtatttcaggtaatcagtatctgtttttgccaccaaatcgttacattttccatggcgctgaggtatattcagactctgaagatgacgtcttatcctctagttcttgtggcagtaacagtgatagtgggacatgccagagtccaagtttagaagaacccatggaggatgaaagtgaaattgaagaattctacaatggcttagaagatgagcctgatgttccagagagagctggaggagctggatttgggactgatggagatgatcaagaggcaattaatgaagctatatctgtgaaacaggaagtaacagacatgaactatccatcaaacaaatcatagtgtaataattgtgcaggtacaggaattgttccaccagcattaggaactttagcatgtcaaaatgaatgtttacttgtgaactcgatagagcaaggaaaccagaaaggtgtaatatttataggttggtaaaatagattgtttttcatggataatttttaacttcattatttctgtacttgtacaaactcaacactaacttttttttttttaaaaaaaaaaaggtactaagtatcttcaatcagctgttggtcaagactaactttcttttaaaggttcatttgtatgataaattcatatgtgtatatataattttttttgttttgtctagtgagtttcaacatttttaaagttttcaaaaagccatcggaatgttaaattaatgtaaagggaacagctaatctagaccaaagaatggtattttcacttttctttgtaacattgaatggtttgaagtactcaaaatctgttacgctaaacttttgattctttaacacaattatttttaaacactggcattttccaaaactgtggcagctaactttttaaaatctcaaatgacatgcagtgtgagtagaaggaagtcaacaatatgtggggagagcactcggttgtctttacttttaaaagtaatacttggtgctaagaatttcaggattattgtatttacgttcaaatgaagatggcttttgtacttcctgtggacatgtagcaatgtctatattggctcataaaactaacctgaaaaacaaataaatgctttggaaatgtttcagttgctttagaaacattagtgcctgcctggatccccttagttttgaaatatttgccattgttgtttaaatacctatcactgtggtagagcttgcattgatcttttccacaagtattaaactgccaaaatgtgaatatgcaaagcctttctgaatctataataatggtacttctactggggagagtgtaatattttggactgctgttttccattaatgaggagagcaacaggcccctgattatacagttccaaagtaataagatgttaattgtaattcagccagaaagtacatgtctcccattgggaggatttggtgttaaataccaaactgctagccctagtattatggagatgaacatgatgatgtaacttgtaatagcagaatagttaatgaatgaaactagttcttataatttatctttatttaaaagcttagcctgccttaaaactagagatcaactttctcagctgcaaaagcttctagtctttcaagaagttcatactttatgaaattgcacagtaagcatttatttttcagaccatttttgaacatcactcctaaattaataaagtattcctctgttgctttagtatttattacaataaaaagggtttgaaatatagctgttctttatgcataaaacacccagctaggaccattactgccagagaaaaaaatcgtattgaatggccatttccctacttataagatgtctcaatctgaatttatttggctacactaaagaatgcagtatatttagttttccatttgcatgatgtttgtgtgctatagatgatattttaaattgaaaagtttgttttaaattatttttacagtgaagactgttttcagctctttttatattgtacatagtcttttatgtaatttactggcatatgttttgtagactgtttaatgactggatatcttccttcaacttttgaaatacaaaaccagtgttttttacttgtacactgttttaaagtctattaaaattgtcatttgacttttttctgttaacttacattgtttaaggtatataatttttaagtcttacagaatttgtacagcttcgaatgctatagtattagatgctttctttctcatactacatttcttcatttggtttattacattagtctcaccagaccagtacttgcaaaaccctgatggtgctgatcgtagcaggtgatgcatcaccaccaagcatttcaaaaactatttcagccattctaggctagaggcctgagcaagtaaaacatggagaaaaggcagggcagggcctaagaaggatgtgacatgtcctatgataaaggaccaggagtttgggatttgagagagaacaggaagtggtggatagtgaaaatccatctgatgcaaaggctgggttaactgaaaatctgttctgggatgctaggtctttcaagtctgaagactgcagaccacatttggtatcaactttctgaggctgacctgagaacctacttaagccattaaaggttttgagggcgaaagtattactgaagaggccagcagtgtaccccttaaaatgtatgtgtaacttggtcgggtgcagtggctcacgcctgtaatcccagcactctgggaggcctacacgggcggaccacctgaggtcaggagttcaagaccagcctggccaacatggcgaaaccccgtttttactaaaaatacaaaaattagtcgggtgtggtggcggcacctgtagtcccagctatttgggaggctgaggctggagaatcgcttcaacccaggaggtggaggctgcagtgagccaagatcgcgccactgcactccagcctggtgacagagcaagactccgtctcaaagaacaaaaaagtatatgtaatgaataaagctttagcaatttcctgttcccagttctgcctccctccccagcaaactttctaggtagacctgtcctgtatattgttagcactagagaaattacctgtaagatgatatcctcaaaatattacaaaattaaggaacattctatatatgcacaaattgaagaagtcaggtagttgatacttttaaaggtacatagtcaatttaagtggtcaaaaggtatttggcaaaacctaatttaactcaagccctatgcataacgttattgctgaaccactggctaatgcagcatgtactgtatttcactgattgtaataccctaattaatgtcacatggtttaattagcgtttttttttgtagtggtacataaaatgacggtggaccttcaatcaatggtgttttacataatccccaactaagctttatttgaaacacaacttctgacctgtaatctagaaaggggtgggggagtatgtatatgtatgtacaccaaaacctctcccccaaagatctaaagccattctaggccttcagctcaaactaggatactagtagaaagtacagaaaaatggtctaatgacctccccaccttttgaagtgactgtcaaatgagactattaaaaaccacttgaaaacttttaaaaagcaagaatagcctgagacctcctagaaatacagatttaattggtgtaaggtggggtttgtgcagagtattaaaaacgcccccaggcgattcaaaagtatagccagatttgagaaacacccttagctggaaaagaattgaggagctctgttttgatgacttttaacatacactccttagggctggcttaaaaaaggaggttaacttacattagcctcaattcccaaattttacctagaaatgcttcataaattaggtaatttggggttcataagcaaatacagcagagtagggagttatcaaaatagagtgaattcttcagcaaaggaggacaaaactaccataaggcattctggatggtcaaagtgcaaaagaggttggggagtggaggaagagactgatgacaggaatattttgctcttgttttagcaggcttaccatgcactcttcaggggaagactgctctatgccatattacaccttgattcacagctttactaaatgtagtaaaaagctgtaaggaagagcaacctgatagtaaaaattgaccccaatgaatggtccttaggaacagatcctatgagaaggctaaccgtgggaatctaatttaggtctctcagtttttacacctacattctggttttattgttttatttatttttatttttttgagacaagtcttgctctgtcgcccaggctggagtgcagtagcgccatctgagctcactgcaacgtccgcctcccaggttcaagtgattctcctgcctcagcctcccgagtagctgggagtacaggcatctgccaccatgcacggctaattttttgtatttttagtagagagagagagggtttcaatatgttggccaggctggtctcgaactcctagactcaagccatctgcccacctcagccttccaagtgctgggattataggtgtaagccaccacgcccagcctgcattctggttttatttccttttaattgaaaggagtgtcttggttctgctgtcttattaatcatcctgaacaatggctaatagcttgaatgactactaagacaggagagctacacacacactatgtatttgtaccttacaaatagatgtgtggaggccaaatacttcagtatttagccaactcaataatcatagtccctacaatttagcaaatgatctgcagccttgctgctctgatctacaaagctcgagacttcttcttgttttttaaaagtaagtggcaatttcagaaatcttcaatggtattttaactttctaccatgaatctccttgctgttaatacattgtatcacagttctggagtaacagggaggaaaagtccccaaattaaactttgtacagtgtgctcccagatgctattctcttgttaaaatatttgttcatggaactattaatccaaagtgcctgctatttcacaaatcagcttcataatatacaaaggttactactattgagaaagagcatctgacatgcttttgatttgaataattactacctcaagaagtggatgaacagggaaaatggtataatatcaggtatgtggaaacactctcatggggaagaatgctactactttgagacaaagtaccttaggtgagaatattgttttagtgcttctatctatagaagagttctatggttcgccaatagttttttaagttaatctactgaaaagtacaagtccacaatcccttatccaaccccttgggcttatttgcattttggaattgtgaattctgaagaattttttaaagcacccaatagcggtcatagtcttatttagtgaataccataaatagcttcaacaattcagatcaggtttttctgcaaagtgagagatgacaatattcttttcagtttttttaaattttagaattgcaggttaagagactgtggacctctataaattcatgtaggaatagaaacaagtcatccgaaatttaagagaccttccctgaaccatccctctttttgaaaaattttgactcttatatgagaaccactgaaagtttaaatattaaaacaataaatctttattaaacagttttcacggcattttaaaataaatttaaaaaataaatatttgcattatagcagaaagtcacatgtattgcatcaactcacttttacaatttaatggcatgagacaactaagcatcagcaccataaaactgttaagatttgtttcttaaattcaaggaaatagctctaaagcactgcagcatcaataattttgaaagctataacccagtgattggtgctcatagtagccaatgtttctttttattactggcaactgtggagacagagttcttccagtactaaatattaataagttctctgatcgacaatcccccatctatcagtgtgtcaggattcacaaagaaaagaactgcaggttatgtagtatattccctatctgtgatttaggtttgcacaaagacacttattctagcaaaaaagaaaagatagtgtttccaccatgctaccttttttttctttttatagagtctcagagtaagatttaaatcataatttttaacctttaaaagtctgttcaagttcaattacaaccatagaaactgtactggtatcagaacataactattttattacaaaacttaacattatttacaaaatgaaaaaataatcaaatgactattgcaggccaaagttaaaggtttttcactcaatgattgaagaaaaattaagcaatatttccatgcactcacaccagatcatttctgaaatatgcaaactcttaaaattcatgttagtaaaactttaatgtattcataatacttgctatgtttattagaagatggtcaaaaaaaatccatggttctgtacaataatattaacagtttgttcattttcctttaatattttttggcttccatgaacactcgtagattgaacattctgcaagtaagaattataatagtacctctgtcaccttgctgaattcatcaccacaagaaaaacacaaatagtttaatgcttttgcactaaactgaatagttataactccaaagttatattaaactgaagccttcattgtgatcaatagcttggatcagtttagagcgtagagtttctttttctgtatattttggaagatccagaagattaaaacaagtatgggaaactgggagatactcctcaccacctcctgtggactggatgactagtttcagactcttcataccaagaataggaatgcgatcactacctgtcaaaaatactgccaagaaagaaatcattcagtcaaccacttcaaaacaaaaagatacagtagcaagtaatatttggtacagtcgctacagcagagcttcactttaacatctgctctaacgtgttatcttacttcctaatttgtggaagacaagttttttgagacacattctaattgctcatccacacaatattttttctttaaaagtataatcttaatactggcacaaaggaagtgcttaataaatgtttgataagtgagtattttgccattgccaaaacattttgagagcccttttttggaaaagccattcagaactagttgacaaaccaaatacaaaatgcagttttgattatgatttctttttctatttgcttcgtttctgttattttttttaaatcagaaatttataatgggcagccaaaattcaaatcattcttttttttttgagacgagtcttgctcagttgcccaggctggagtgcagtgtgcggtcacagtcacagcagcctcgacctcctgggctcaagcaatcctctctacctcagcttctggagtagctgggagcacagtcaccaagcccagggctaatttttatagagatgaggttttgccatgttgcccaggctcaaatcactcttaaagagtaatgatttctcatccttatgacacacagaagaatttgaccaagattctcggaggttgccaaatcattcctggcaaaagcaggagtatcaaaggaaatgtcaccgatttggacacataaattctgacaactttaaaaataagtcaaatttcaattagaatacagactttaacagaagaaagatacacaggcatcagttataaatgagggaactaaggcctgaaaaagctttattttatatagaaattgccaaagttttagtcttgaagcaaaaagaaaaagaaaaagaaaatgaagaccaaacagagaaaagagcaatacagttggccctccgtatctgtgagctctgtatccatggattgaaccgactgcaaatcgaaaatattttcaaaaaatggatgcttgtgactgtactgaatgtgtcaactgtttttcttgacattggtccctcaacaatacagtataacaactatttacatagcatttacattgtatgaggtattaataacatgtacattcatgtgtcacttaacgatgatgatatgttctgagaaatgcatcattaggtgatttcattctgtaaacatcatatatttacacaacctagacagtatatagtctactacacacctaggctacatgatatagccctattgctcctaggctacaaacctgtacagcatgttactgtactgaattctgtaggtaactgtaacataacagtaggtatttgtgtatctaaacatagaaaaggtacagtagctgtattataatcttacaggaccactatcgtatatatggtctgtcattcactgaaaagtcattatgtggcacatgactgtaatctagagaagatttaaagtataaggatatgtgtaggttatatgcaaatactatgccaatttatacaagaaacttgagcatctactggatttgggtatccttggcagggaggtcttggaatcaatcccccatgtatgccaaaggatgactatactccagagatctttttagaaatctaaaaggaattctacaatcctagtttattgtaaccacagtttattgtaactataacatagctatagtttgctttcatttccaagttagcttccattttacttcatagaatgaatcaatgctattttgaaggcaaactgaatttagcctttgaaataagaaaatacatgtactgaagagtgcagatagttctgtttcaaaaaaattacttgaagattaaaacagggctttggaataatactataattagattccagtctcataaagtatacaactagttagaaatacttacacagaaactgtttcttcttttccaatggtaattcgtgaaatacttcccaaaaaatttttatcgtaggatgttctgcccaatattcccctttgtattctgtattctaaaaacaacataatcttttattagtattaagggacacactggctaatatattagcacataaagctaacatggtccagtagtttgcaacttgtgcaattcaatggattgcccactgttttctggacatttacaatttgaagctattagttccaggagaaaaagaagaaatacgtaagaatcaccagaaatctttcctagcacttcaaagaacatgataggatcatactatcatcttcaaaagtaaataaagagaaactagataaaataatgttataggaccatcaaaagtctgacatggaattatctgtgataatcagaaaattccctttctctgtattcatgaacagaagtcaaaccattacaatttatctctccttttaaaaatatggtactaagcaacactaaaaattttctgaggtagtttccaagatggccaccagtgagcactgcctcctggtattcatgcccgtgtgtataaggatactgtgaatatgaggaatgtatgacttctgagactaaatcataaaaaaaaaaacctctatggcttcagcccttcttctggatcactcactctgggggaaaccagttgtcatttcatgaggatattcaaacatccctatatggagagaagtactcatggtgctatactgaggcctcctgccaacagtcatgtgagtaagccaccttggaagtgaatctctatccttaatcatgcttttagatgcctatagcaccagccaatattctgactgcaacttcatgagggaaccctgagcccaaactactcagctaagccactcctgaattcctgacccacagaaactatgaaacaagagtttcattgttattttgaagccaataagttttggggtaatttgctgcaataggactaaaatatctcccctgtcaaaaatattagtactggccgggcgcggtggctcacgcctataatcccagcactttgggtgtccaagccgggcagatcacttgaggtcaggagttccagaccagcctggccaacatggtgaaaccccatctctactgaaaatacaaaaattagctgggcatggtggcaggcgcctgtaatcccggctacttgggggaggctaaggcaggagaatcgcttgaacttgggagacggaggttgtagtgagccaagactgtgccattgcactccagcctggtcaacaagagcgaaactccatctcaataaaaaaaataaaaaataaaaaaaaaaaaaaacaaaaaaattagtactaaaagagaagcaggtaggaagtagagaggaaatagtttatactgtgaactgttcttatccagccgcagttggctttaactttcagttcttcagatgcgtcaggtcctttggacatactgttccctcagcctacaccactcagacacatcctttcttctttggtcatcaggctaatttttacccttttagggctcagattaaatgccatctaattcattcattgattcatccaacaaatatataatgagttttagtcaatgttttgggccctgggaaaacctcagtgaacagaatcctcacttaccttgtggagctcaccgtctagtaagaggcgttcccagattcatctagcctagatcaggctcctccttacgcaccattcactaaactctttttcactgtattaattaaaactgtgtattaaacatttaatttctagctctcttattagggcaaagaccatgtctctatcttatttcctgccctctcttcaatctcttgcatggtttcagctgtacattatccggcacacagtagaggctcaataaatacttgctaatgggcttcttgaattttggactttccttatttgtagccatgatttatcttaatcactgaactctcaataattcaaatatgcaaaatatttgctgttagttgcacacaagtttgaagaaaacagcttgaattctagcatggaatttatggaatttattcaactgtttgaattaaagtctgtgagcatgttagtggggaatgaaaagaggatggctaaagtttttttttcttttaaatctctaaatggtttggtagaaatgtgcacacatgtcctgattaagaaagaacatttaaagctccaaatcataatcactaaaattgtaatgaatcagatatacaacagaagaaatcaaagtgttaagaataaatacaccatatatatatatatatatatatatatatatatatatatatatatatatatatttttttttttttttagatagagtcttgctctgtcaccaggctggaatacagtggcacgatcttggctcactgcaacctctgcctcccaggttcaagcgattctcctgcctcagcctcccgagtagctgggattacaggcctacaccaccacacccagctaattttcgtatttttagtagagacgggggtttcactatgttggccaggatggtcttgatcttctgacctcgtgatccgcctgcctcagcctcccaaagtgctgggattacaggcatgagccactgcgcccggccgctacaatatactttttaagatgtcctgtcttaactgaatgaacacttaaatcaattatcacttcaggcaaggtttcaagtacttactgagtgctctcctaggtgctgggaatacagc